(19) United States
(12) Patent Application Publication
(10) Pub. No.: US 2024/0132479 A1
(43) Pub. Date: Apr. 25, 2024

(54) THERAPEUTIC COMPOUNDS AND USES THEREOF

(71) Applicant: Board of Trustees of Michigan State University, East Lansing, MI (US)

(72) Inventors: Robert B. Abramovitch, East Lansing, MI (US); Edmund Ellsworth, Vicksburg, MI (US); John Williams, Lansing, MI (US)

(21) Appl. No.: 18/274,705

(22) PCT Filed: Jan. 28, 2022
(86) PCT No.: PCT/US2022/014342

§ 371 Date: Jul. 27, 2023

Related U.S. Application Data
(60) Provisional application No. 63/143,583, filed on Jan. 29, 2021.

(51) Int. Cl. C07D 403/06; A61P 31/04; C07D 235/16; C07D 235/26; C07D 235/28; C07D 401/04; C07D 401/06; C07D 401/12; C07D 403/04

(52) U.S. Cl. CPC C07D 403/06; A61P 31/04; C07D 235/16; C07D 235/26; C07D 235/28; C07D 401/04; C07D 401/06; C07D 401/12; C07D 403/04

(57) ABSTRACT

A liner in a label-liner combination is provided with a die cut portion made in the liner. The die cut portion is aligned with at least one edge in a label and a front side of the liner is attached to a backside of the label. The die cut portion of the liner is adapted to be removed from the liner when the label is removed from the label-liner combination. Dimensions, and orientation, and a location of the die cut portion in the liner are adapted to allow the label to be removed from the label-liner combination and applied to a surface by digits of a hand without touching an adhesive coating on the backside of the label and adapted to maintain proper printer waste liner spool operations when the liner is wound after application of the label.

### CROSS-REFERENCE TO RELATED APPLICATIONS

This application claims the benefit of U.S. Provisional Appl. Ser. No. 63/143,583, filed Jan. 29, 2021, which is incorporated by reference as if fully set forth herein.

### STATEMENT OF GOVERNMENT SUPPORT

This invention was made with government support under AI057153, AI117018, and AI116605 awarded by the National Institutes of Health. The government has certain rights in the invention.

### BACKGROUND

Tuberculosis (TB) is a disease caused by the bacterium Mycobacterium tuberculosis (Mtb). The disease is spread from person to person through the air. It is estimated that one-third of world's population is latently infected by Mtb. Despite the availability of effective anti-TB drugs, such as isoniazide and rifampin, TB is still one of the world's deadliest diseases. According to World Health Organization, there were 9.4 million new TB cases and 1.7 million people died from TB in 2009, [Global tuberculosis control: WHO report 2010.WHOl/HTMITB/2010.7].

Nontuberculous mycobacteria (NTM) are ubiquitous in the environment and include more than 90 different species, causing colonization, infection, and pseudo-outbreaks in health care settings. Data suggest that the frequency of nosocomial outbreaks due to NTM may be increasing and reduced hot water temperatures may be partly responsible for this phenomenon. Attention to adequate high-level disinfection of medical devices and the use of sterile reagents and biologics will prevent most outbreaks. Because NTM cannot be eliminated from the hospital environment and they present an ongoing potential of infection, NTM should be considered in all cases of nosocomial infection, and careful surveillance must be used to identify potential outbreaks.

Development of new agents that reduce the duration and complexity of current therapies, as well as effectively kill emerging resistant mutants, multi-drug resistant TB and/or NTM and extensively drug resistant TB and/or NTM, would have a major impact on TB and/or NTM therapy and healthcare in countries where tuberculosis and NTM are prevalent.

### SUMMARY

The instant disclosure provides, among other things, new agents that reduce or eliminate at least one of the duration and complexity of current therapies, as well as effectively kill emerging resistant mutants, multi-drug resistant TB and/or NTM and extensively drug resistant TB and/or NTM. The new agents include, for example, compounds of the formula (I) and (I′):

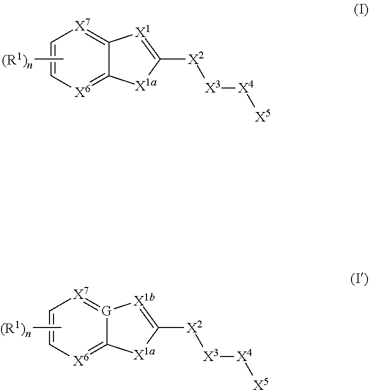

or a pharmaceutically acceptable salt, polymorph, prodrug, solvate or clathrate thereof wherein:
each R¹ is independently H, halo, alkyl, alkoxy, aryl, aryloxy, amino, S(O)ₙR¹, nitro, cyano, heterocyclyl or two R¹ groups on adjacent atoms can form an aryl or a heterocyclyl group together with the atoms to which they are attached;
n is 0, 1, or 2;
X¹ and X^{1a} are each independently O, N or NR⁴, wherein R⁴ is H, alkyl, cycloalkyl or aryl;

### G is N or C;

### X^{1b} is N or CH;

X² is alkyl, alkenyl, O, NR^{4a}, C(O) or S(O)ₓ, wherein x is 0, 1 or 2 and R^{4a} is H, alkyl, cycloalkyl cr aryl;
X³ is absent, alkyl or NR⁴;
X⁴ is C(O) or alkyl;
X⁵ is alkyl, cycloalkyl, heterocyclyl or NR²R³, wherein R² and R³ can each independently be H, alkyl, cycloalkyl, aryl or R² and R³, together with the nitrogen atom to which they are attached, form a heterocyclyl group;
or wherein X²-X⁴, together with substituents attached thereto, form a 3-5-membered ring;
X⁶ and X⁷ are each independently N or CR⁵, wherein R⁵ can be H or R¹.

### DESCRIPTION OF THE FIGURES

The drawings illustrate generally, by way of example, but not by way of limitation, various embodiments discussed in the present document.

FIG. 1 is a plot of lung burden as a function of treatment.

FIG. 2 is a plot of spleen burden as a function of treatment.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

The instant disclosure generally relates to compounds of the formula (I) and (I′):

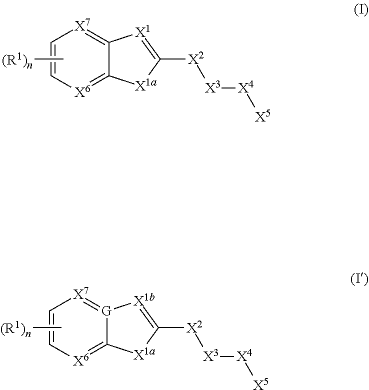

or a pharmaceutically acceptable salt, polymorph, prodrug, solvate or clathrate thereof wherein:
each R¹ is independently H, halo, alkyl, alkoxy, aryl, aryloxy, amino, S(O)ₙR¹, nitro, cyano, heterocyclyl or two R¹ groups on adjacent atoms can form an aryl or a heterocyclyl group together with the atoms to which they are attached;
n is 0, 1, or 2;
X¹ and X^{1a} are each independently O, N or NR⁴, wherein R⁴ is H, alkyl, cycloalkyl or aryl;

### G is N or C;

### X^{1b} is N or CH;

X² is alkyl, alkenyl, O, NR^{4a}, C(O) or S(O)ₓ, wherein x is 0, 1 or 2 and R^{4a} is H, alkyl, cycloalkyl or aryl;
X³ is absent, alkyl or NR⁴;
X⁴ is C(O) or alkyl;
X⁵ is alkyl, cycloalkyl, heterocyclyl or NR²R³, wherein R² and R³ can each independently be H, alkyl, cycloalkyl, aryl or R² and R³, together with the nitrogen atom to which they are attached, form a heterocyclyl group;
or wherein X²-X⁴, together with substituents attached thereto, form a 3-5-membered ring;
X⁶ and X⁷ are each independently N or CR⁵, wherein R⁵ can be H or R¹.

The types of ring systems that can be formed by the groups:

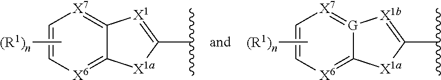

include, but are not limited to the following:

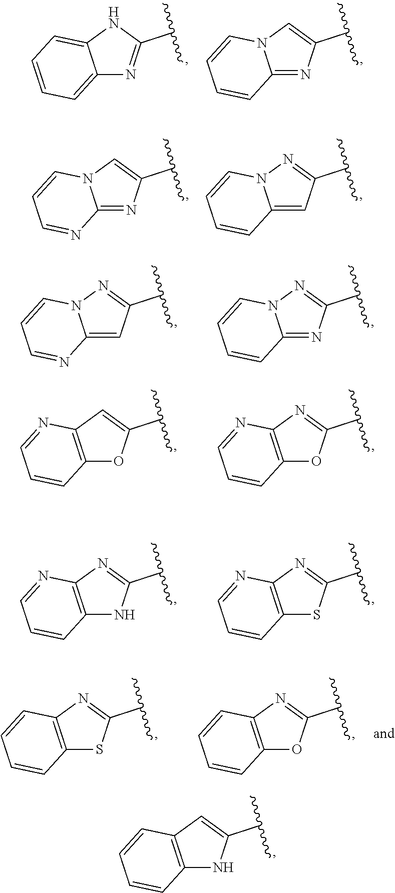

such as:

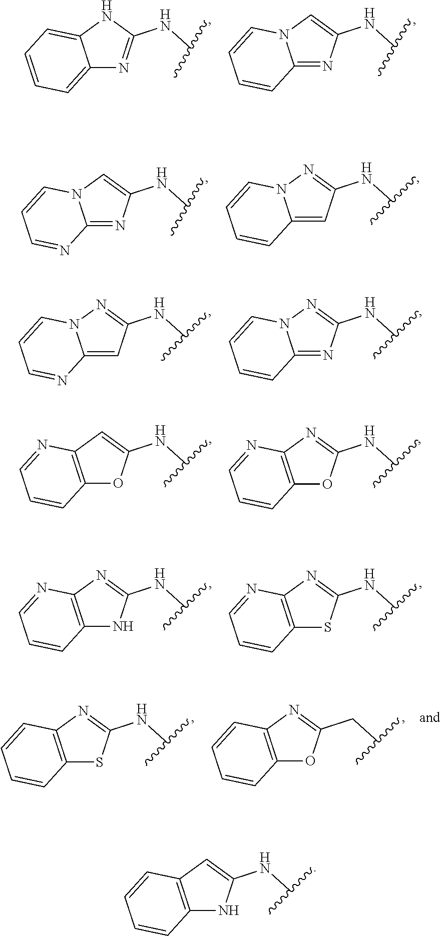

In the compounds of the formula (I) and (I′), X² can be S(O)ₜ, wherein t can be 1 or 2. Alternatively, X² can be S. Alternatively, X² can be NR^{4a}. Alternatively, X² can be O. Alternatively, X² can be C(O). Alternatively, X² can be alkyl (e.g., substituted or unsubstituted alkyl such as methylene (CH₂) and alkyl substituted alkyl, such as isopropyl). Alternatively, X² can be an unsaturated group, such as alkenyl (e.g., substituted or unsubstituted alkenyl, such as ethylenyl (—CH═CH—) and alkyl substituted alkenyl, such as —CH═C(CH₃)—) or alkynyl (e.g., CH₂—C≡C—).

In addition to, or alternatively to the foregoing recitations for X², X³ can be absent or alkyl. For example, X³ can be (C₁-C₆)alkyl.

In addition to, or alternatively to the foregoing recitations for X² and/or X³, X⁴ can be acyl, such as C(O). Thus, for example, X² can be alkyl and X⁴ can be C(O) or X² and X³ can be alkyl and X⁴ can be C(O).

In some examples, X²-X⁵, together with substituents attached thereto, form a 3-5-membered ring. Thus, for example, X² can be NR^{4a}, X³ can be alkyl (e.g., CH₂), X⁴ can be C(O), and X⁵ can be alkyl (e.g., CH₂) so as to form a five-membered ring of the formula:

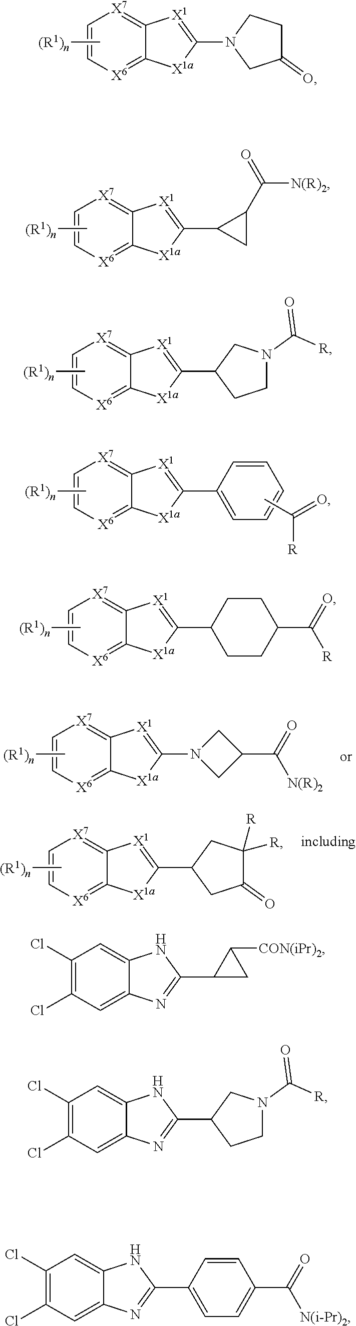

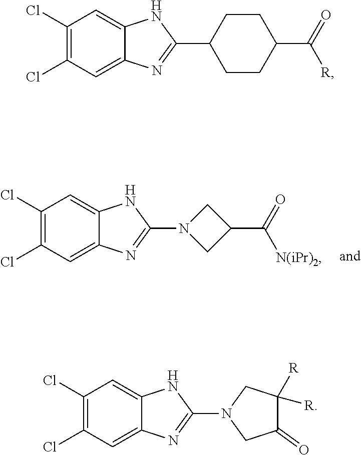

wherein, in instances described herein, the group R^{4a} can be alkyl (e.g., CH₂) and “participates” in the formation of the ring,

In addition to, or alternatively to the foregoing recitations for X², X³, and/or X⁴, X⁵ can be cycloalkyl or NR²R³. For example, X⁵ can be NR²R³. In another example, X⁵ can be NR²R³ and R² and R³, together with the nitrogen atom to which they are attached, form a heterocyclyl group. Or, when X⁵ can be NR²R³, at least one of R² and R³ can be aryl. Or, when X⁵ can be NR² R³ and at least one of R² and R³ can be cycloalkyl.

Examples of compounds of the formula (I) and (I′) include compounds of the formulae (a)-(c):

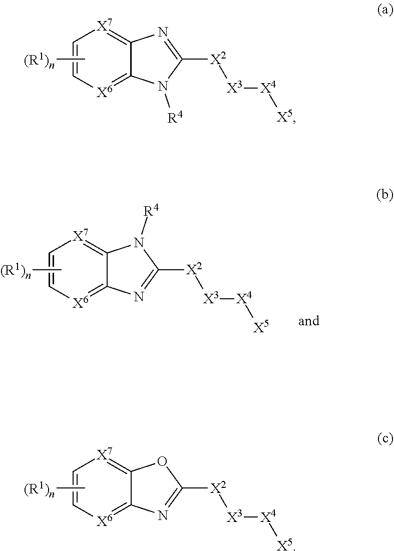

In the compounds of formulae (a)-(c) and formula (I) and (I′), R⁴ can be alkyl. In addition to, or alternatively, X² can be S(O), and X³ can be alkyl. For example, X² can be S. In the compounds of formulae (a)-(c) and formula (I) and (I′), the X² can be alkyl and X³ can be absent. Or X² can be alkenyl and X³ can be absent. Or X² can be alkyl and X³ can be N⁴. Or X² can be NR^{4a} and X³ can be alkyl. Or X² can be NR^{4a} and X³ can be absent. Or X² can be O and X³ can be alkyl. Or X² can be C(O) and X³ can be absent.

In addition to, or alternatively, in the compounds of formulae (a)-(c) and formula (I) and (I′), at least one of X⁶ and X⁷ can be N. Or X⁶ can be CR⁵ and X⁷ can be N, wherein R⁵ can be H or R¹. Or X⁶ and X⁷ can be CR⁵, wherein R⁵ can be H or R¹. Or X⁶ and X⁷ can each independently be CR⁵. Also contemplated herein are compounds of the formula (II):

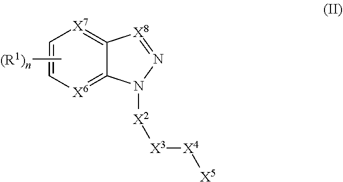

or a pharmaceutically acceptable salt, polymorph, prodrug, solvate or clathrate thereof wherein:
R¹, n, and X²-X⁷ are as defined for the compound of formula (I) and (I′) herein; and X³ is N or CR^{4b}, wherein R^{4b} is H, alkyl or aryl.

In the compounds of the formula (II), X² can be S(O)ₜ, wherein t can be 1 or 2. Alternatively, X² can be S. Alternatively, X² can be NR⁴ a. Alternatively, X² can be O. Alternatively, X² can be C(O). Alternatively, X² can be alkyl (e.g., substituted or unsubstituted alkyl such as methylene (CH₂) and alkyl substituted alkyl, such as isopropyl). Alternatively, X² can be an unsaturated group, such as alkenyl (e.g., substituted or unsubstituted alkenyl, such as ethylenyl (—CH═CH—) and alkyl substituted alkenyl, such as —CH═C(CH₃)—) or alkynyl (e.g., CH₂—C≡C—).

In addition to, or alternatively to the foregoing recitations for X², in the compounds of formula (II), X³ can be absent or alkyl. For example, X³ can be (C₁-C₆)alkyl.

In addition to, or alternatively to the foregoing recitations for X² and/or X³, in the compounds of formula (II), X⁴ can be acyl, such as C(O). Thus, for example, X² can be alkyl and X⁴ can be C(O) or X² and X³ can be alkyl and X⁴ can be C(O).

In some examples, X²-X⁵ in the compounds of formula (II), together with substituents attached thereto, form a 3-5-membered ring. Thus, for example, X² can be alkyl, X³ can be alkyl (e.g., CH₂), X⁴ can be C(O), and X⁵ can be alkyl (e.g., CH₂) so as to form a five-membered ring of the formula:

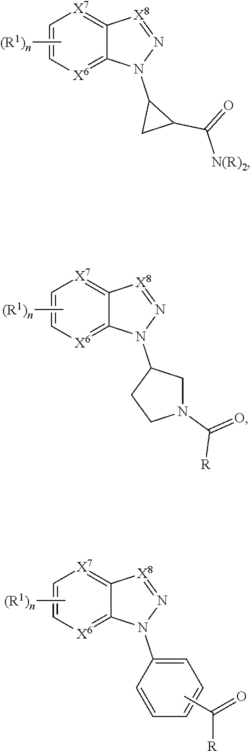

wherein, in instances described herein, the group R^{4a} can be alkyl (e.g., CH₂) and “participates” in the formation of the ring.

In the compounds of formula (II), in addition to, or alternatively to the foregoing recitations for X², X³, and/or X⁴, X⁵ can be cycloalkyl or NR²R³. For example, X⁵ can be NR²R³. In another example, X⁵ can be NR²R³ and R² and R³, together with the nitrogen atom to which they are attached, form a heterocyclyl group. Or, when X⁵ can be NR²R³, at least one of R² and R³ can be aryl. Or, when X⁵ can be NR²R³ and at least one of R² and R³ can be cycloalkyl.

Compounds of the formula (I) and (I′) include compounds of the formulae:

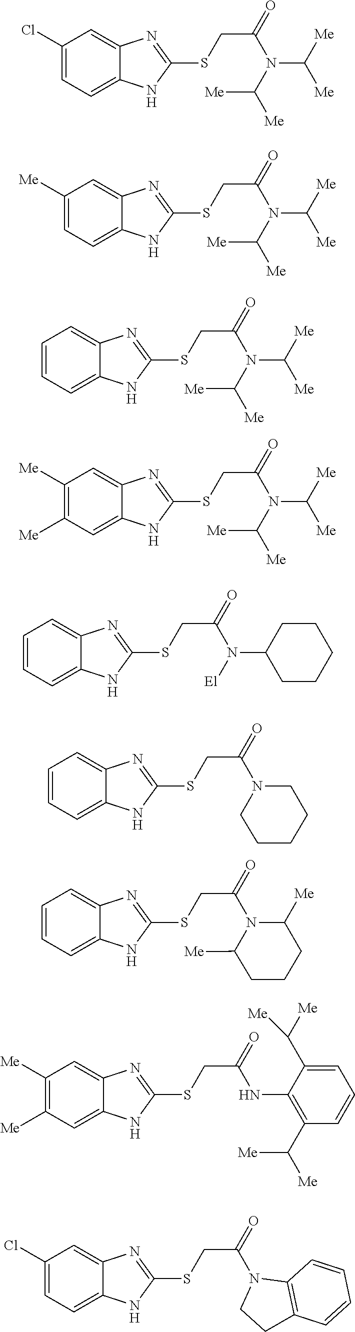

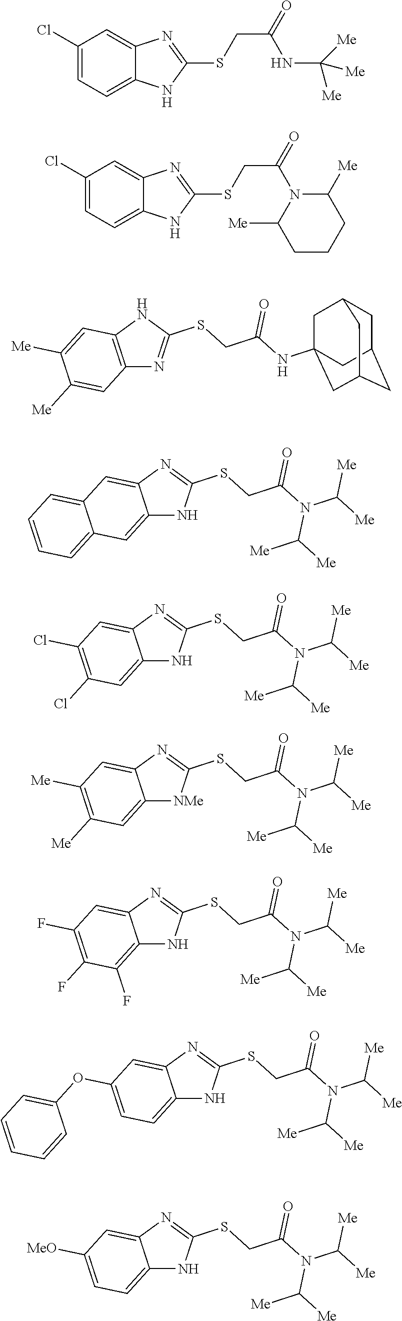

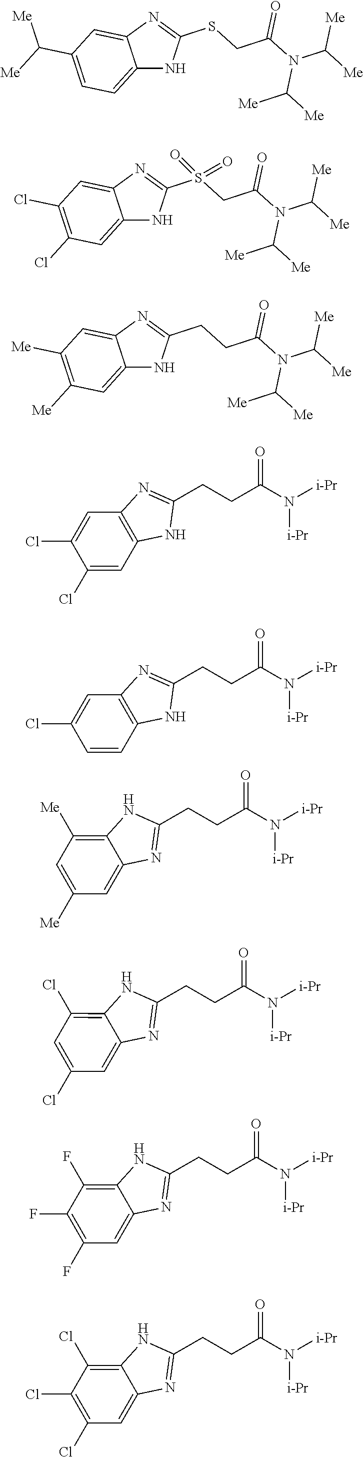

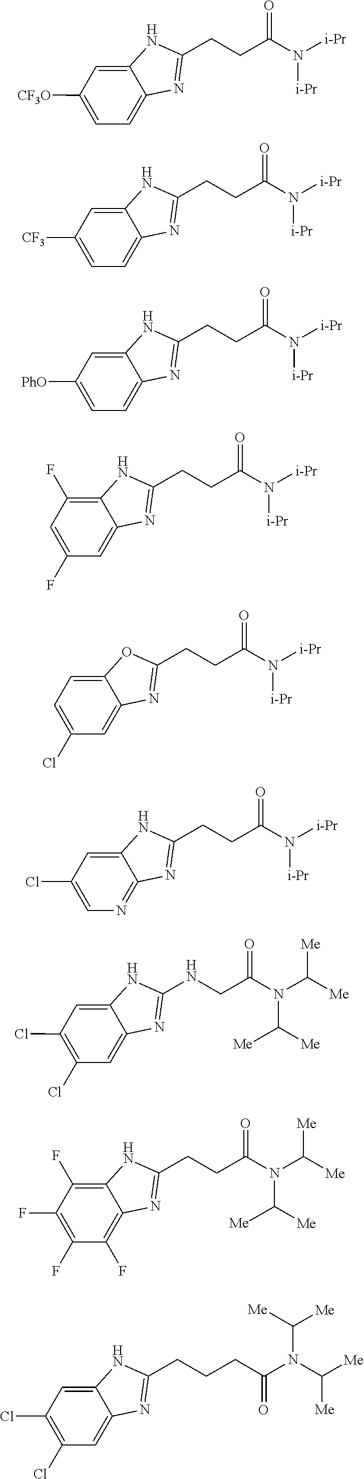

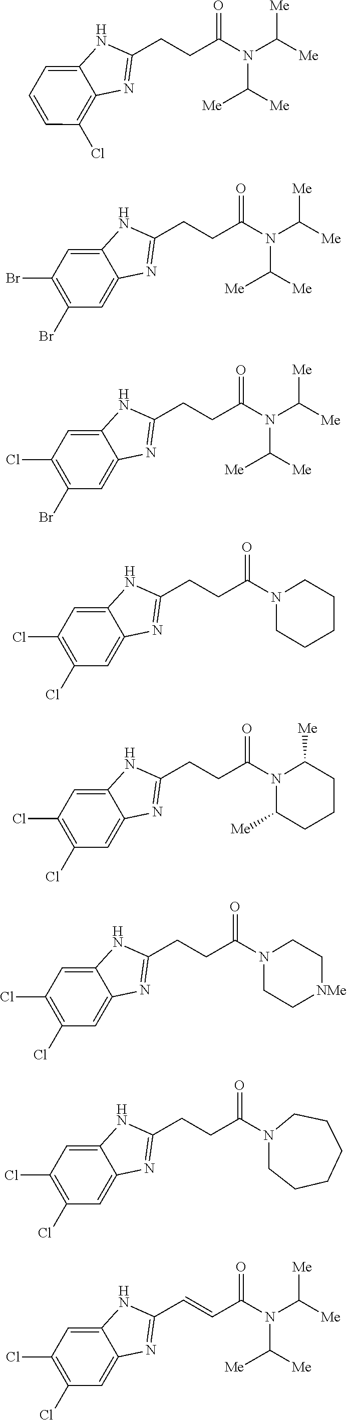

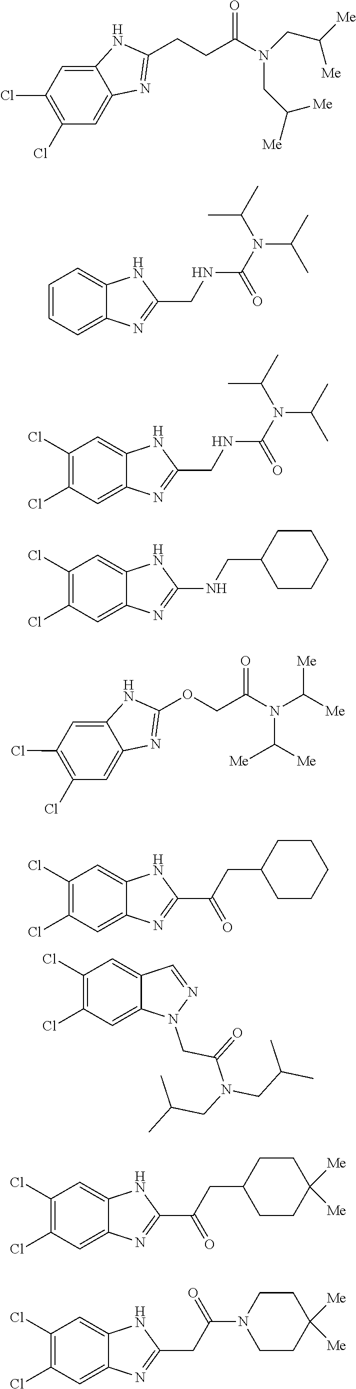

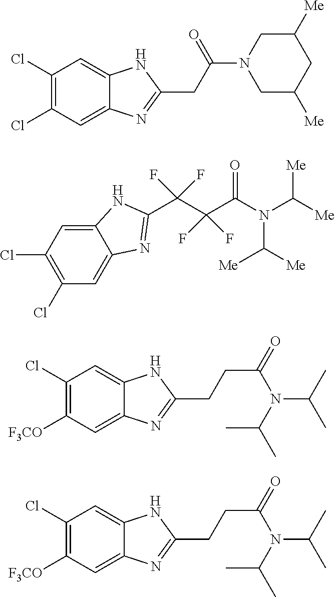

Also contemplated herein are compounds of the formula (III):

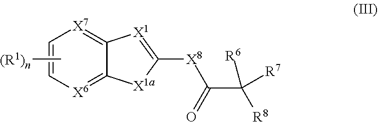

or a pharmaceutically acceptable salt, polymorph, prodrug, solvate or clathrate thereof wherein:
each R¹ is independently H, halo, alkyl, alkoxy, aryl, aryloxy, amino, S(O)ₙR¹, nitro, cyano, heterocyclyl or two groups on adjacent atoms can form an aryl or a heterocyclyl group together with the atoms to which they are attached;
n is 0, 1, or 2;
X¹ and X^{1a} are each independently O, N or NR⁴, wherein R⁴ is H, alkyl, cycloalkyl or aryl;
X⁶ and X⁷ are each independently N or CR⁵, wherein R⁵ can be H or R¹;
X⁸ is NR^{4a}, wherein R^{4a} is H, alkyl, cycloalkyl or aryl;
R⁶ and R⁷ are each, independently, alkyl or, together with the carbon atom to which they are attached, form a cycloalkyl or a heterocyclyl; and
R⁸ is alkyl, alkoxy, aryl or heteroaryl.

Also contemplated herein are compounds of the formula (IV)

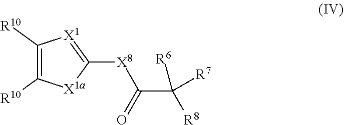

or a pharmaceutically acceptable salt, polymorph, prodrug, solvate or clathrate thereof wherein:
X¹ and X^{1a} are each independently O, N or NR⁴, wherein R⁴ is H, alkyl, cycloalkyl or aryl;
X⁶ and X⁷ are each independently N or CR⁵, wherein R⁵ can be H or R¹;
X⁸ is NR^{4a}, wherein R^{4a} is H, alkyl, cycloalkyl or aryl;
R⁶ and R⁷ are each, independently, alkyl or, together with the carbon atom to which they are attached, form a cycloalkyl or a heterocyclyl;
R⁹ is alkyl (e.g., methyl and ethyl), haloalkyl (e.g., CF₃), alkoxy, haloalkoxy (e.g., OCF₃), aryloxy, cycloalkyl (e.g., (C₃-C₆) cycloalkyl and (C₃-C₅) cycloalkyl, such as cyclopropyl, cyclobutyl, cyclopentyl and cyclohexyl) or heteroaryl; and
each R¹⁰ is, independently, alkyl, aryl or arylalkyl.

In the compounds of the formulae (I), (II), and (III), X¹ and X^{1a} can be NH⁴ and N, respectively, such as NH and N, respectively; or X¹ and X^{1a} can be O and N, respectively. Alternatively or in addition, X⁶ and X⁷ are each, independently, CH or N, such that the ring comprising X⁶ and X⁷ are each CH; X⁶ is CH and X⁷ is N; X⁶ is N and X⁷ is CH; or X⁶ and X⁷ are each CH.

In the compounds of the formula (IV), X¹ and X^{1a} can be NR⁴ and N, respectively, such as NH and N, respectively; or X¹ and X^{1a} can be O and N, respectively.

Examples of compounds of the formula (IV) include, but are not limited to compounds of the formulae:

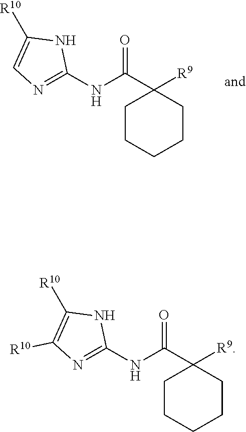

Pharmaceutical compositions are also contemplated herein, comprising one or more compounds of described herein (e.g. a compound of the formula (I) and (I′)) and one or more pharmaceutically acceptable carriers, diluents, excipients or combinations thereof. A “pharmaceutical composition” refers to a chemical or biological composition suitable for administration to a subject (e.g., mammal). Such compositions may be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” comprises a carrier, sometimes a liquid, in which an active therapeutic agent is formulated. The excipient generally does not provide any pharmacological activity to the formulation, though it may provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes any and all solvents, dispersion media, coatings, antibacterial and antifungal agents, isotonic and absorption delaying aaents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions may be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In some cases isotonic agents can be included in the pharmaceutical compositions, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems, Biodegradable, biocompatible polymers may be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions may be orally administered as a capsule (hard or soft), tablet (film coated, enteric coated or uncoated), powder or granules (coated or uncoated) or liquid (solution or suspension). The formulations may be conveniently prepared by any of the methods well-known in the art. The pharmaceutical compositions may include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients; such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various embodiments may vary according to factors such as the disease state, age, gender, weight, patient history, risk factors, predisposition to disease, administration route, pre-existing treatment regime (e.g., possible interactions with other medications), and weight of the individual. Dosage regimens may be adjusted to provide the optimum therapeutic response. For example, a single bolus may be administered, several divided doses may be administered over time, or the dose may be proportionally reduced or increased as indicated by the exigencies of therapeutic situation.

“Dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals. In therapeutic use for treatment of conditions in mammals (e.g., humans) for which the compounds of the various embodiments described herein or an appropriate pharmaceutical composition thereof are effective, the compounds of the various embodiments described herein may be administered in an effective amount. The dosages as suitable for this invention may be a composition, a pharmaceutical composition or any other compositions described herein.

The dosage can be administered once, twice, or thrice a day, although more frequent dosing intervals are possible. The dosage may be administered every day, every 2 days, every 3 days, every 4 days, every 5 days, every 6 days, and/or every 7 days (once a week). In one embodiment, the dosage may be administered daily for up to and including 30 days, preferably between 7-10 days. In another embodiment, the dosage may be administered twice a day for 10 days. If the patient requires treatment for a chronic disease or condition, the dosage may be administered for as long as signs and/or symptoms persist. The patient may require “maintenance treatment” where the patient is receiving dosages every day for months, years, or the remainder of their lives. In addition, the composition of this invention may be to effect prophylaxis of recurring symptoms. For example, the dosage may be administered once or twice a day to prevent the onset of symptoms in patients at risk, especially for asymptomatic patients.

The compositions described herein may be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to the cell, tissue, organ, and/or organ system, which encompasses and/or is affected by the disease, and/or where the disease signs and/or symptoms are active or are likely to occur, Administration can be topical with a local effect, composition is applied directly where its action is desired. Administration can be enteral wherein the desired effect is systemic (non-local), composition is given via the digestive tract. Administration can be parenteral, where the desired effect is systemic, composition is given by other routes than the digestive tract.

Compositions comprising a therapeutically effective amount of one or more compounds of the various embodiments described herein (e.g. a compound of the formula (I) and (I′)) are also contemplated. The compositions are useful in a method for treating at least one of tuberculosis and an NTM infection, the method comprising administering one or more compounds described herein (e.g., a compound of formula (I) and (I′)) to a subject in need of treatment of at least one of tuberculosis and an NTM infection. Also contemplated herein is one or more compounds described herein for use as a medicament for treating a patient in need of relief from at least one of tuberculosis and an NTM infection.

The term “therapeutically effective amount” as used herein, refers to that amount of one or more compounds of the various embodiments described herein (e.g. a compound of the formula (I) and (I′)) that elicits a biological or medicinal response in a tissue system, animal or human, that is being sought by a researcher, veterinarian, medical doctor or other clinician, which includes alleviation of the symptoms of the disease or disorder being treated (e.g., tuberculosis). The therapeutically effective amount can be that which may treat or alleviate the disease or symptoms of the disease at a reasonable benefitirisk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process,

The term “substituted,” “substituent,” and “functional group,” as used herein refers to a group that can be or is substituted onto a molecule or onto another group (e.g., on an aryl or an alkyl group). Examples of substituents include, but are not limited to, a halogen (e.g., F, Cl, Br, and I), OR, OC(O)N(R)₂, ON, NO, NO₂, ONO₂, azido, CF₃, OCF₃, R, O(oxo), S (thiono), C(O), S(O), methylenedioxy, ethylenedioxy, N(R)₂, SR, COR, SO₂R, SO₂N(R)₂, SO₃R, —(CH₂)₀₋₂P(O)(OR)₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, (R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein each R can be, independently, hydrogen, halo, alkyl, acyl, cycloalkyl, aryl, aralkyl, heterocyclyl, heteroaryl, or heteroaryialkyl, wherein any alkyl, acyl, cycloalkyl, aryl, aralkyl, heterocyclyl, heteroaryl, or heteroaryialkyl or two R groups, together, can form a cycloalkyl group or two R groups bonded to a nitrogen atom or to adjacent nitrogen atoms can together with the nitrogen atom or atoms form a heterocyclyl, which can be mono- or independently multi-substituted.

The term “alkyl” as used herein refers to substituted or unsubstituted straight chain and branched mono- or divalent alkyl groups and cycloalkyl groups having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups, to form dihalo or trihaloakyl groups, including difluoromethyl and trifiuoromethyl groups.

The term “alkenyl” as used herein refers to substituted or unsubstituted straight chain and branched mono- or divalent alkenyl groups and cycloalkenyl groups having at least one double bond and having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkenyl groups include those with from 1 to 3 carbon atoms such as —CH′CH—, —CH═CHCH₃, and —CH₂CH═CHCH₂— groups, wherein the double bonds can have an E- or Z-configuration. And when there are multiple bonds, each double bond can, independently, have an E- or a Z-configuration. Examples of branched alkenyl groups include, but are not limited to, —CH═C(CH₃)— and CH₂C—CH(CH₃) groups. Representative substituted alkenyl groups can be substituted one or more times with any of the groups listed herein, for example, amino, hydroxy, cyano, carboxy, nitro, thio, alkoxy, and halogen groups.

The term “cycloalkyl” as used herein refers to substituted or unsubstituted cyclic alkyl groups such as, but not limited to, cyclopropyl, cyciobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups can have any number of carbon atoms, e.g., 3 to 8 carbon atoms (C₃-C₈), 3 to 6 carbon atoms (C₃-C₆), and 4 to 8 carbon atoms (C₄-C₈). Cycloalkyl groups further include polycyclic cycloalkyl groups such as, but not limited to, norbornyl, adarnantyl, bornyl, camphenyl, isocamphenyl, and carenyl groups, and fused rings such as, but not limited to, decalinyl, and the like.

The term “cycloalkylalkyl” as used herein refers to substituted or unsubstituted alkyl groups as defined herein in which a hydrogen or carbon bond of an alkyl group as defined herein is replaced with a bond to a cycloalkyl group as defined herein. Representative cycloalkylalkyl groups include, but are not limited to, cyclopentylalkyl.

The term “alkylcycloalkyl” as used herein refers to substituted or unsubstituted cycloalkyl groups as defined herein in which a hydrogen of a cycloalkyl group as defined herein is replaced with a bond to an alkyl group as defined herein. Representative alkylcycloalkyl groups include, but are not limited to, alkylcyciopropyl.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heteracyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyi, and acryloyl groups and the like. When the group containing the carbon atom that is bonded to the carbonyl carbon atom contains a halogen, the group is termed a “haloacyl” group. An example is a trifluoroacetyl group.

The term “heterocyclylcarbonyl” is an example of an acyl group that is bonded to a substituted or unsubstituted heterocyclyl group, as the term “heterocyclyl” is defined herein. An example of a heterocyclyicarbonyl group is a prolyl group, wherein the ,prolyl group can be a D- or an L-prolyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptaienyl, biphenyl, indacenyl, fluorenyi, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 8-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” or “heterocyclo” as used herein refers to substituted or unsubstituted aromatic and non-aromatic ring compounds containing 3 or more ring members, of which, one or more (e.g., 1, 2 or 3) is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₅), 3 to 6 carbon atoms (C₃-C₆), 3 to 5 carbon atoms (C₃-C₅) or 6 to 8 carbon atoms (C₆-C₈). A heterocyclyl group designated as a C₂-heterocyciyl can be a 5-ring with two carbon atoms and three heteroatoms, a 6-ring with two carbon atoms and four heteroatoms and so forth. Likewise a C₄-heterocyclyi can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic aroups. Representative heterocyclyl groups include, but are not limited to pyrrolidinyl, azetidinyl, piperidynyl, piperazinyl, niorpholinyl, chromanyl, indolinonyl, isoindolinonyl, uranyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

Examples of indolinonyl groups include groups having the general formula:

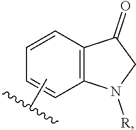

wherein R is as defined herein.

Examples of isoindolinonyl groups include groups having the general formula:

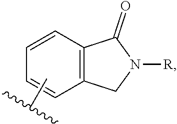

wherein R is as defined herein.

Examples of benzoxazolinyl groups include groups having the general formula:

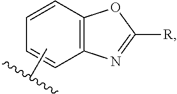

wherein R is as defined herein.

Examples of benzthiazolinyl groups include groups having the general formula:

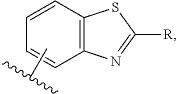

wherein R is as defined herein.

In some embodiments, the group R in benzoxazolinyl and benzthiazolinyl groups is an N(R)₂ group. In some embodiments, each R is hydrogen or alkyl, wherein the alkyl group is substituted or unsubstituted. In some embodiments, the alkyl group is substituted with a heterocyclyl group (e.g., with a pyrrolidinyl aroup).

The term “heterocyclyialkyl” as used herein refers to alkyl groups as defined herein in which a hydrogen or carbon bond of an alkyl group as defined herein is replaced with a bond to a heterocyclyl group as defined herein. Representative heterocyclylaikyl aroups include, but are not limited to, furan-2-yl methyl, furan-3-yl methyl, pyridine-3-yl methyl, tetrahydrofuran-2-yi methyl, and indo1-2-yl propyl.

The term “heterocyclylalkoxy” as used herein refers to alkyl groups as defined herein in which a hydrogen or carbon bond of an alkyl group as defined herein is replaced with a bond to a heterocyclyl group as defined herein and the alkyl group is attached to an oxygen. Representative heterocyclylalkoxy groups include, but are not limited to, —O—(CH₂)_{q} heterocyclyl, wherein q is an integer from 1 to 5. In some embodiments, heterocyclylalkoxy groups include —O—(CH₂)_{q} morpholinyl such as —O—CH₂CH₂-morpholine.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The terms “amine,” “amine group,” “amino,” and “amino group” as used herein refer to a substituent of the form —NH₂, —NHR, —NR₂, —NR₃⁺, wherein each R is defined herein, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group.

An “alkylamino” group includes a monoalkylamino, dialkylamino, and trialkylamino group. An example of a “alkylamino” is —NH-alkyl and —N(alkyl)₂.

An example of a “cycloalkylamino” group is —NH-cycloalkyl and —N(cycloalkyl)₂.

An example of a “cycloalkyl heterocycloamino” group is —NH-(heterocyclo cycloalkyl), wherein the heterocyclo group is attached to the nitrogen and the cycloalkyl group is attached to the heterocyclo group.

An example of a “heterocyclo cycloamino” group is —NH-(cycloalkyl heterocycle), wherein the cycloalkyl group is attached to the nitrogen and the heterocyclo group is attached to the cycloalkyl group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein ail halo atoms can be the same or different, and per-halo alkyl groups, wherein ail hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

As used herein, the term “salts” and “pharmaceutically acceptable salts” refer to derivatives of the disclosed compounds wherein the parent compound is modified by making add or base salts thereof. Examples of pharmaceutically acceptable salts include, but are not limited to, mineral or organic add salts of basic groups such as amines; and alkali or organic salts of acidic groups such as carboxylic adds. Pharmaceutically acceptable salts include the conventional non-toxic salts or the quaternary ammonium salts of the parent compound formed, for example, from non-toxic inorganic or organic adds. For example, such conventional non-toxic salts include those derived from inorganic adds such as hydrochloric, hydrobromic, sulfuric, sulfamic, phosphoric, and nitric; and the salts prepared from organic acids such as acetic, propionic, succinic, glycolic, stearic, lactic, mak, tartaric, citric, ascorbic, pamoic, maleic, hydroxymaleic, phenylacetic, glutamic, benzoic, salicylic, sulfanilic, 2-acetoxybenzoic, fumaric, toluenesulfonic, niethanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z.” has the same meaning as “about X, about Y, or about Z,” unless indicated otherwise.

In this document, the terms “a,” “an,” or “the” are used to include, one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in theft entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carded out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0,5%, 0,1%, 0,05%, 0.001%, or at less than about 0.0005% or less or about 0% or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

### EXAMPLES

The disclosure can be better understood by reference to the following examples which are offered by way of illustration. The disclosure is not limited to the examples given herein.

### Example 1

### 2-(1H-1,3-benrodiazol-2-ylsulfanyl)-N,N-bis(propan-2-yl)acetamide

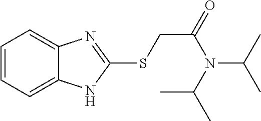 [SMILES: CC(C)N(C(=O)CSc1nc2ccccc2[nH]1)C(C)C]

### 5-Thia-2,7-diazatricyclo[6.4.0.0^{2,6}]dodeca-1(12),6,8,10-tetraen-3-one

The title compound was prepared by the following method (Mavrova, A. Ts.; Anichina K. K.; Vuchev, D.; Tsenov, J. A.; Denkova, P. S.; Kondeva, M. S.; Micheva, M. K., Eur. J. Med. Chem., 2006, 41, 1412). A 25.0 mL 14/20 round bottom flask was charged with 2-(1H-1,3-benzodiazol-2-ylsulfanyl)acetic acid (0.208 g, 1.00 mmol) and pyridine (0.600 mL). The suspension was heated to 80° C. and treated with acetic anhydride (0.200 mL). The reaction mixture clears and after 10 minutes a precipitate formed. The stirring mixture was cooled to room temperature and water added (2.00 mL). The resulting solid was filtered and dried to a constant weight in an oven, yielding 5-thia-2,7-diazatricyclo[6.4.0.0^{2.6}]dodeca-1(12),6,8,10-tetraen-3-one (0.155 g, 82%). ¹H NMR (500 MHz, DMSO-d₆) δ 7.90-7.83 (m, 1H), 7.59 (dt, J=8.0, 1.0 Hz, 1H), 7.37 (td, J=7.7, 1.3 Hz, 1H), 7.32 (td, J=7.6, 1.2 Hz, 1H), 4.61 (s, 2H).

### 2-(1H-1,3-benzodiazol-2-ylsulfanyl)-N,N-bis(propan-2-yl)acetamide

To a 10-dram vial was added toluene (2,00 mL), diisopropylamine (0.202 g, 2.00 mmol) and 5-thia-2,7-diazatricyclo[6.4.0.0^{2.6}]dodeca-1(12),6,8,10-tetraen-3-one (0.090 g, 0.47 mmol). The mixture was heated to 70° C. and stirred for 22 hours to provide a solid, which was filtered, washed with hexanes, and dried in vacuo to provide 2-(1H-1,3-benzodiazol-2-ylsulfanyl)-N,N-bis(propan-2-yl)acetamide. (0.012 g, 9%), NMR (500 MHz, Chloroform-d) δ7.47 (dd, J=6.1, 3.1 Hz, 2H), 7.17 (dd, J=6.0, 3.2 Hz, 2H), 3.60 (s, 2H), 3.34 (p, J=6.5 Hz, 2H), 1.34 (d, J=6.5 Hz, 12H). APCI [M+H] calc'd=292.1478, observed=292.1485.

### Example 2

### 2-[(5,6-dimethyl-1H-1,3-benzodiazol-2-yl)sulfanyl]-N,N-bis(propan-2-yl)acetamide

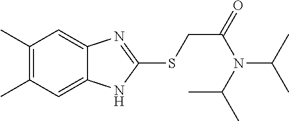 [SMILES: Cc1cc2nc(SCC(=O)N(C(C)C)C(C)C)[nH]c2cc1C]

2-chloro-N,N-bis(propan-2-yl)acetamide. The title compound was prepared by the following method (Chen, T. C.; Yu, D. S.; Fu, Y. C.; Lee, C. C.; Chen, C. L.; Huang, F. C.; Hsieh, H. H.; Lin, J. J.; Huang, H. S., Eur. J. Med. Chem, 2013, 69, 278). A 100 mL 24/40 round bottom flask (argon atmosphere) was charged with dichloromethane (20.0 mL) and chloroacetyl chloride (1.12 g, 10.0 mmol) and cooled to 0° C. Diisopropylamine (2.52 g, 25.0 mmol), dissolved in dichloromethane (10.0 mL), was added (0° C.) to the rapidly stirring solution, in a dropwise fashion. resulting in a precipitate. The mixture was stirred for 30 minutes at 0° C., then treated with 30.0 mL of a 1.00 N aqueous hydrochloric acid solution. The biphasic reaction mixture was stirred for 5 minutes at 0° C., then transferred to a separatory funnel and partitioned. The aqueous layer was washed with 15.0 mL dichloromethane and combined with the organic layer. The organic mixture was washed with brine, dried over sodium sulfate, filtered, and concentrated in vacuo to yield an oil, which was filtered through a plug of silica (100% dichloromethane). The filtrate was concentrated in vacuo to yield the pure product as a clear, colorless oil (1.51 g, 85% yield). ¹H NMR (500 MHz, CDCl₃) δ4.01 (s, 2H), 3.95 (p, J=6.7 Hz, 1H), 3.43 (dd, J=13.1, 6.9 Hz, 1H), 1.39 (d, J=6.8 Hz, 6H), 1.24 (d, J=6.7 Hz, 6H).

5,6-dimethyl-1H-1,3-benzodiazole-2-thiol. A 100 mL 24/40 round bottom flask was charged with ethanol (20.0 mL), carbon disulfide (1.67 g, 22.0 mol) and solid sodium hydroxide (0.880 g, 22.0 mmoles). 4,5-dimethylbenzene-1,2-diamine (2.58 g, 19.0 mmol) was added, followed by 3.00 mL water. The reaction mixture (argon atmosphere) was heated to reflux for 3 hours and a precipitate formed. The reaction mixture was cooled to room temperature, filtered, and washed with ethanol. The filtrate was diluted with 20.0 mL water, heated to 70° C. with stirring, and treated with 10.0 mL of a 1:1 acetic acid and water mixture, resulting in the formation of a precipitate. The mixture was cooled to 0° C. for 3 hours, filtered, and dried in vacuo for 20 hours to yield a solid (2.50 g, 74%, prepared according to the method described by Peddibhotla, S.; Shi, R.; Kahn, P.; Smith, L. H.; Mangravita-Novo, A.; Vicchiarelli, M.; Su, Y.; Okolotowica, K. J.; Cashman, J. R.; Reed, J. C.; Roth, G. P.; J. Med. Chem., 2010, 53, 4793.). ¹H NMR (500 MHz, CDCl₃) δ 7.47 (dd, J=6.0, 3.2 Hz, 1H), 7.17 (dd, J=6.0, 3.1 Hz, 1H), 3.61 (s, 1H), 3.33 (h, J=6.5 Hz, 1H), 1.34 (d, J=6.5 Hz, 6H).

2-[(5,6-dimethyl-1H-1,3-benzodiazol-2-yl)sulfanyl]-N,N-bis(propan-2-yl)acetamide. A 50.0 mL round bottom flask was charged with 2-chloro-N,N-bis(propan-2-yl)acetamide (0.354 g, 2.00 mmol), dry dimethylformamide (2.00 mL), and 5,6-dimethyl-1H-1,3-benzodiazole-2-thiol (1.00 mmol, 0.178 g). The reaction mixture was stirred, under an argon atmosphere, and treated with triethylamine (0.252 g, 2.50 mmol). After 18 hours, the mixture was poured into water (50.0 mL), and the resulting solid filtered, washed with water and hexanes, then dried in vacuo to provide 2-[(5,6-dimethyl-1H-1,3-benzodiazol-2-yl)sulfanyl]-N,N-bis(propan-2-yl)acetamide (0.161 g, 50%). ¹H NMR (500 MHz, DMSO-d₆) δ 12.23 (s, 1H), 7.24 (s, 1H), 7.11 (s, 1H), 4.35 (s, 2H), 4.06 (p, J=6.6 Hz, 1H), 3.52-3.45 (m, 1H), 2.25 (d, J=6.7 Hz, 6H), 2.07 (s, 2H), 1.27 (d, J=6.7 Hz, 6H), 1.17 (d, J=6.6 Hz, 6H). APCI HRMS [M+H] calc'd=320.1791, found 320.1790. m.p=172° C.

### Example 3

### 2-(1H-1,3-benzodiazol-2-ylsulfanyl)-N-cyclohexyl-N-ethylacetamide

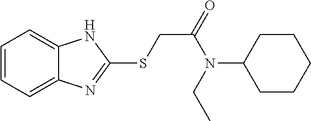 [SMILES: CCN(C(=O)CSc1nc2ccccc2[nH]1)C1CCCCC1]

2-(1H-1,3-benzodiazol-2-ylsulfanyl)-N-cyclohexyl-N-ethylacetamide. A 10-dram vial was charged with toluene (2.00 mL), N-ethyl-N-cyclohexylamine (0.254 g, 2.00 mmol) and the 5-thia-2,7-diazatricyclo[6.4.0.0^{2.6}]dodeca-1(12),6,8,10-tetraen-3-one (0.105 g, 0.55 mmol). The mixture was heated to 70° C. with stirring for 22 hours, The resulting solid was filtered, washed with hexanes and dried in vacuo to yield the pure product (0.051 g, 29%). ¹H NMR (500 MHz, Chloroform-d) δ 9.64 (s, 2H), 7.48 (dd, J=6.0, 3.2 Hz, 2H), 7.17 (dd, J=6.0, 3.1 Hz, 2H), 3.67 (s, 2H), 2.98 (q, J=7.2 Hz, 2H), 2.86 (tt, J=11.7, 3.9 Hz, 1H), 2.05 (dd, J=12.4, 3.8 Hz, 2H), 1.74 (dt, J=13.3, 3.5 Hz, 2H), 1.60 (dt, J=13.0, 3.3 Hz, 1H), 1.42 (dd, J=12.3, 3.5 Hz, 1H), 1.37 (dd, J=12.4, 3.6 Hz, 1H), 1.29 (t, J=7.2 Hz, 3H), 1.19 (qt, J=12.9, 3.2 Hz, 2H), 1.08 (ddt, J=16.2, 12.7, 6.2 Hz, 1H). APCI HRMS [M−H] calc'd=316.1489, found 316.1436.

### Example 4

### 2-(1H-1,3-benzodiazol-2-ylsulfanyl)-1-(piperidin-1-yl)ethan-1-one

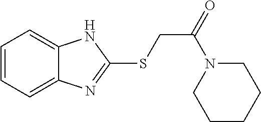 [SMILES: O=C(CSc1nc2ccccc2[nH]1)N1CCCCC1]

A 10-dram vial was charged with toluene (2.00 mL), piperidine (2.00 mmol, 0.170 g, 0.200 mL) and 5-thia-2,7-diazatricyclo[6.4.0,0^{2.6}]dodeca-1 (12),6,8,10-tetraen-3-one (0.090 g, 0.470 mmol). The mixture was heated to 70° C., stirred for 20 hours, concentrated, then redissolved in toluene (5.00 mL). The mixture was then washed with water, dried over sodium sulfate, filtered and concentrated in vacuo to yield the product as an oil (0.039 g, 14%) ¹H NMR (500 MHz, Chloroform-d) δ 7.54 (t, J=4.9 Hz, 2H), 7.24-7.16 (m, 2H), 3.98 (s, 1H), 3.68-3.62 (m, 1H), 3.51 (t, J=5.1 Hz, 1H), 1.73-1.65 (m, 2H), 1.64-1.57 (m, 1H). APCI [M+H] calc'd=276.1165, observed=276.1169.

### Example 5

### 2-(1H-1,3-benzodiazol-2-ylsulfanyl)-1-(2,6-dimethylpiperidin-1-yl)ethan-1-one

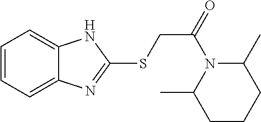 [SMILES: CC1CCCC(C)N1C(=O)CSc1nc2ccccc2[nH]1]

A 10-dram vial was charged with chlorobenzene (2.00 mL), piperidine (3.46 g, 27.0 mmol) and 5-thia-2,7-diazatricyclo[6.4.0.0^{2.6}]dodeca-1(12),6,8,10-tetraen-3-one (0.070 g, 0.36 mmol). The mixture was heated to 130° C. and stirred for 24 hours. The mixture was subsequently cooled to room temperature and washed with water, brine, and the organic layer dried over sodium sulfate. The organic layer was filtered and concentrated in vacuo to yield the product as an oil (0.004 g, 1.30%). ¹H NMR (500 MHz, Chloroform-d) δ 7.48 (t, J=4.7 Hz, 1H), 7.21-7.14 (m, 1H), 3.64 (s, 1H), 3.00 (q, J=6.5 Hz, 1H), 1.87-1.77 (m, 1H), 1.80-1.73 (m, 1H), 1.61-1.52 (m, 1H), 1.55-1.39 (m, 1H), 1.34 (d, J=6.3 Hz, 4H). APCI [M+H] calc'd=304.1478, observed=304.1505.

### Example 6

### N[2,6-bis(propan-2-yl)phenyl]-2-[(5,6-dimethyl-1H-1,3-benzodiazol-2-yl)sulfanyl]acetamide

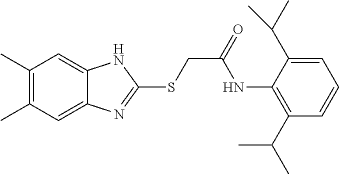 [SMILES: Cc1cc2nc(SCC(=O)Nc3c(C(C)C)cccc3C(C)C)[nH]c2cc1C]

A 50.0 mL 24/40 round bottom flask was charged with N-[2,6-bis(propan-2-yl)phenyl]-2-chloroacetamide (0.761 g, 3.00 mmol, Thomas, R. M,; Keitz, B. J.; Champagne, T. M.; Grubbs, R. H,; J. Am, Chem. Soc., 2011, 133, 7490), dimethylformamide (5.00 mL) and 5,6-dimethyl-1H-1,3-benzodiazole-2-thiol (0.356 g, 2.00 mmol) under an argon atmosphere. Triethylamine (0.505 g, 5.00 mmol) was added and the mixture stirred at room temperature for 17 hours. Water was added (200 mL) and a solid formed, which was filtered, washed with water and dried in vacuo. The product was purified by trituration from hot ethanol to yield a solid (0.252 g, 30%). ¹H NMR (500 MHz, DMSO-d₆) δ 12.38 (s, 1H), 9.68 (s, 1H), 7.25 (s, 1H), 7.20 (t, J=7.7 Hz, 1H), 7.14 (s, 1H), 7.08 (d, J=7.7 Hz, 2H), 4.18 (s, 2H), 3.02 (hept, J=6.9 Hz, 2H), 2.26 (d, J=6.1 Hz, 6H), 0.98 (s, 11H). APCI [M+H] calc'd=396.2104, observed=396.2117.

### Example 7

### 2-[(6-chloro-1H-1,3-benzodiazol-2-yl)sulfanyl]-1-(2,3-dihydro-1H-indol-1-yl)ethan-1-one

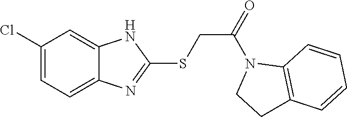 [SMILES: O=C(CSc1nc2ccc(Cl)cc2[nH]1)N1CCc2ccccc21]

2-chloro-1-(2,3-dihydro-1H-indol-1-yl)ethan-1-one. A 50.0 mL 24/40 round bottom flask was charged with indoline (1.19 g, 10.0 mmol) and dichloromethane (10.0 mL), then cooled to 0° C. under an argon atmosphere, and treated dropwise with a solution of chloroacetyi chloride (0.564 g, 5.0 mmol) dissolved in 0.600 mL dichloromethane. A solid formed and additional dichloromethane (10.0 mL) was added. The reaction mixture was stirred at 0° C. for 1 hour then washed with saturated sodium bicarbonate, dried over sodium sulfate, filtered, and concentrated to a solid (0.783 g, 40%). ¹H NMR (500 MHz, DMSO-d₆) δ8.02 (d, J=8.0 Hz, 1H), 7.24 (d, J=7.4 Hz, 1H), 7.16 (t, J=7.7 Hz, 1H), 7.02 (td, J=7.5, 1.1 Hz, 1H), 4.52 (s, 2H), 4.11 (t, J=8,5 Hz, 2H), 3.15 (t, J=8,4 Hz, 2H).

6-chloro-1H-1,3-benzodiazole-2-thiol. A 100 mL 14/20 roundbottom flask was charged with 4-chloro-1,2-diaminobenzene (2.70 g, 19.0 mmol), water (2.00 mL), ethanol (20.0 mL), sodium hydroxide (0.880 g, 22.0 mmol) and carbon disulfide (1.67 g, 22.0 mmol). The mixture was heated to reflux for 3 hours under an argon atmosphere, then cooled to 70° C. and treated with 10.0 mL of a 1:9 mixture of acetic acid:water. A precipitate formed, the reaction mixture and cooled at 0° C. for 3 hours. The precipitate was then filtered and washed with a 1:1 ethanol:water mixture. The solid was dried in vacuo to yield the product as a powder which was used without further purification (2.00 g, 57%). ¹H NMR (500 MHz, DMSO-d₆) δ 12.46 (s, 2H), 7.11 (d, J=14.5 Hz, 3H).

2-[(6-chloro-1H-1,3-bermodiazol-2-yl)sulfanyl]-1-(2,3-dihydro-1H-indol-1-yl)ethan-1-one. A 50.0 mL 24/40 round bottom flask was charged with 6-chloro-1H-1,3-benzodiazole-2-thiol (0.184 g, 1.00 mmol), dimethylformamide (2.00 mL), and 2-chloro-1-(2,3-dihydro-1H-indol-1-yl)ethan-1-one (0.391 g, 2.00 mmol) The reaction mixture was stirred under an argon atmosphere, then treated with triethylamine (0.505 g, 5.00 mmol) and stirred at room temperature for 18 hours, during which a precipitate formed. Water (15.0 mL) was added and the precipitate filtered, washed with water, and dried to constant weight. The product was purified by trituration from hot ethanol to yield the pure compound as a solid (0.210 g, 75%). ¹H NMR (500 MHz, DMSO-d₆) δ8.05-7.87 (m, 1H), 7.81-7.50 (m, 1H), 7.34-7.10 (m, 3H), 7.09-6.96 (m, 1H), 5.32 (d, J=9.0 Hz, 1H), 4.61-4.48 (m, 1H), 4.37 (t, J=8.4 Hz, 1H), 4.33-4.22 (m, 1H), 3.27 (t, d=8.4 Hz, 1H), 3.24-3.14 (m, 1H), 2.93-2.69 (m, 1H). APCI [M+H] calc'd=344.0618, observed=344.0619.

### Example 8

### N-tert-butyl-2-[(6-chloro-1H-1,3-benzodiazol-2-yl)sulfanyl]acetamide

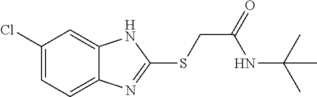 [SMILES: CC(C)(C)NC(=O)CSc1nc2ccc(Cl)cc2[nH]1]

N-tert-butyl-2-chloroacetamide. A 50.0 mL round bottom flask was charged with chloroacetyl chloride (1.12 g, 10.0 mmol) and dichloromethane (15.0 mL), then cooled to 0° C. (argon atmosphere). t-Butylamine (1.82 g, 25.0 mmol) dissolved in dichloromethane (10.0 mL) was added then added dropwise resulting in a precipitate. The mixture was stirred for 30 minutes at 0° C., then 30.0 mL of a 1.0 N aqueous hydrochloric acid solution added. The biphasic mixture was stirred for 5 minutes at 0° C. and partitioned. The aqueous layer was washed with 15.0 mL dichloromethane. The organic layers were then combined, washed with brine, dried over sodium sulfate, filtered, and concentrated in vacuo to yield an oil (1.23 g, 82%). ¹H NMR (500 MHz, DMSO-d₆) δ 3.95 (s, 2H), 1.25 (s, 9H). N-tert-butyl-2-[(6-chloro-1H-1,3-benzodiazol-2-yl)sulfanyl]acetamide. A 50.0 mL round bottom flask was charged with 6-chloro-1H-1,3-benzodiazole-2-thiol (0.184 g, 1.0 mmol) and dimethylformamide (2.00 mL). The mixture was stirred under an argon atmosphere and N-tert-butyl-2-chloroacetamide (0.302 g, 2.00 mmol) added. Triethylamine (0.303 g, 3.00 mmol) was added and the mixture stirred at room temperature for 18 hours. Water (15.0 mL) was added and the mixture extracted with ethyl acetate. The organic layer was dried over sodium sulfate and concentrated in vacuo. The crude material was purified by Reverse-phase Medium Pressure Liquid Chromatography (50 g C18 column, 50-100% methanol in 25.0 mmolar ammonium formate) to provide the formate salt. The salt was dissolved in ethyl acetate, washed with sodium bicarbonate, brine, dried over sodium sulfate, filtered, and concentrated to yield the final product (0.078 g, 26%). ¹H NMR (500 MHz, Chloroform-d) δ 11.49 (s, 1H), 7.77 (s, 1H), 7.51 (s, 1H), 7.41 (d, J=8.5 Hz, 1H), 7.18 (dd, J=8.5, 2.0 Hz, 1H), 3.71 (s, 2H), 1.36 (s, 9H). APCI [M+H] calc'd=298.0775, observed=298.0782.

### Example 9

### 2-[(6-chloro-1H-1,3-benzodiazol-2-yl)sulfanyl]-1-(2,6-dimethylpiperidin-1-yl)ethan-1-one

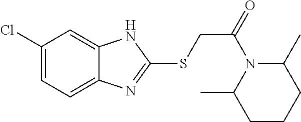 [SMILES: CC1CCCC(C)N1C(=O)CSc1nc2ccc(Cl)cc2[nH]1]

2-chloro-1-(2,6-dimethylpiperidin-1-yl)ethan-1-one. A 50.0 mL round bottom flask was charged with chloroacetyl chloride (1.12 g, 10.0 mmol) and dichloromethane (15.0 mL). The mixture was cooled to 0° C. (argon atmosphere) and treated dropwise with a solution of 2,6-cis-dimethylamine (2.82 g, 25.0 mmol) in dichloromethane (10.0 mL). Precipitation was observed and the mixture stirred for 30 minutes at 0° C., at which time 30.0 mL 1.00 N aqueous hydrochloric acid solution was added. The biphasic reaction mixture was stirred for 5 minutes at 0° C., at which time it was transferred to a separatory funnel and partitioned. The aqueous layer was washed with 15.0 mL dichloromethane. The organic layers were combined, washed with brine, dried over sodium sulfate and concentrated in vacuo to yield a clear oil (1.33 g, 70%. Prepared by the method of Liu, C.; Zhang, M.; Zhang, Z.; Zhang, S. B.; Yang, S.; Zhang, A.; Yin, L.; Swarts, S.; Vidyasagar, S.; Zhang, L.; Okunieff, P.; Bioorg. Med. Chem., 2016, 24, 4263). ¹H NMR (500 MHz, DMSO-d₆) δ4.59 (s, 2H), 4.36-4.17 (m, 1H), 4.08 (s, 1H), 1.77 (tq, J=13.3, 8.3, 6.5 Hz, 1H), 1.66-1.47 (m, 4H), 1.42 (dp, J=13.3, 3.7 Hz, 1H), 1.34-0.98 (m, 6H).

2-[(6-chloro-1H-1,3-benzodiazol-2-yl)sulfanyl]-1-(2,6-dimethylpiperidin-1-yl)ethan-1-one. A 50.0 mL round bottom flask was charged with 6-chloro-1H-1,3-benzodiazole-2-thiol (0.369 g, 2.00 mmol), dimethylformamide (3.00 mL) and 2-chloro-1-(2,6-dimethylpiperidin-1-yl)ethan-1-one (0.568 g, 3.0 mmol). Triethylamine (0.505 g, 5.00 mmol) was added under an argon atmosphere and the mixture stirred at room temperature for 18 hours. Water (15.0 mL) was added and the mixture extracted with ethyl acetate and organic layer concentrated in vacuo. The crude material was purified by reverse-phase medium pressure liquid chromatography (50 g C18 column, 50-100% methanol in 25.0 mmolar ammonium formate). The formate solvate was dissolved in ethyl acetate and washed with a saturated sodium bicarbonate solution, brine, dried over sodium sulfate, filtered, and concentrated in vacuo to yield the pure product as a solid (0.225 g, 33%).¹H NMR (500 MHz, DMSO-d₆) δ 12.71 (s, 1H), 7.59-7.29 (m, 2H), 7.11 (d, J=8.4 Hz, 1H), 4.71-4.43 (m, 2H), 4.39-4.12 (m, 2H), 1.85-1.39 (m, 6H), 1.37-0.99 (m, 5H). APCI [M+H] calc'd=338.1088, observed=338.1091.

### Example 10

### N-(adamantan-1-yl)-2-[(5,6-dimethyl-1H-1,3-benzodiazol-2-yl)sulfanyl]acetamide

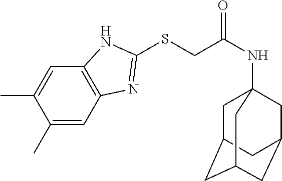 [SMILES: Cc1cc2nc(SCC(=O)NC34CC5CC(CC(C5)C3)C4)[nH]c2cc1C]

N-(adamantan-1-yl)-2-chloroacetamide. A 50.0 mL 24/40 round bottom flask was charged with imidazole (0.680 g, 10.0 mmol) and dichloromethane (10.0 mL) and cooled to 0° C., Chloroacetyl chloride (0.784 g, 7.00 mmol) was added dropwise as a solution in dichloromethane (0.500 mL) providing a precipitate that was stirred for 1 hour at 0° C. 20.0 mL of a 1.00 N aqueous hydrochloric acid solution was added, and the biphasic mixture stirred for 5 minutes at 0° C. and partitioned. The aqueous layer was washed with 15.0 mL dichloromethane, the organic layers combined, washed with brine, dried over sodium sulfate, filtered, and concentrated in vacuo to yield the product as an oil (0.510 g, 45%). ¹H NMR (500 MHz, CDCl₃) δ6.29-6.19 (m, 1H), 3.94 (s, 2H), 2.12-2.07 (m, 3H), 2.01 (d, J=3.0 Hz, 7H), 1.75-1.62 (m, 7H).

N-(adamantan-1-yl)-2-[(5,6-dimethyl-1H-1,3-benzodiazol-2-yl)sulfanyl]acetamide. A 50.0 mL round bottom flask was charged with 5,6-dimethyl-1H-1,3-benzodiazole-2-thiol (0.200 g, 1.10 mmol), dimethylformamide (2.00 mL) and N-(adamantan-1-yl)-2-chloroacetamide (0.510 g, 2.30 mmol). Triethylamine (0.505 g, 5.00 mmol) was added under an argon atmosphere and the mixture stirred at room temperature for 18 hours. Water (15.0 mL) was added and the mixture extracted with ethyl acetate. The organic layer was washed with brine, dried over sodium sulfate, filtered, and concentrated in vacuo. The product was purified by recrystallization from methanol (0.206 g, 51% ¹H NMR (500 MHz, DMSO-d₆) δ 12.33 (s, 1H), 7.99 (s, 1H), 7.22 (s, 1H), 7.14 (s, 1H), 3.87 (s, 2H), 2.25 (s, 6H), 1.97 (dd, J=5.8, 3.1 Hz, 3H), 1.87 (d, J=2.9 Hz, OH), 1.58 (s, 5H), APCI [M+H] calc'd=370.1947, observed=370.1955.

### Example 11

### 2-{1H-naphtho[2,3-d]imidazol-2-ylsulfanyl}-N,N-bis(propan-2-yl)acetamide

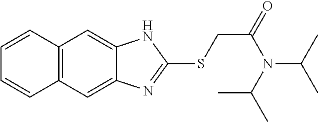 [SMILES: CC(C)N(C(=O)CSc1nc2cc3ccccc3cc2[nH]1)C(C)C]

1H-naphtho[2,3-d]imidazole-2-thiol. A 50.0 mL round bottom flask was charged with 2,3-diaminonaphthalene (2.37 g, 15.0 mmol), ethanol (20.0 mL), water (2.00 mL) and sodium hydroxide (0.719 g, 18.0 mmol). The mixture was subsequently heated to 75° C. for 3 hours after being treated with carbon disulfide (1.37 g, 18.0 mmol). The reaction was quenched by adding water (20.0 mL) and cooled to room temperature. The product was precipitated by adding 10.0 mL of a 1:9 acetic acid:water mixture, the solid filtered, washed with hexanes, and dried in vacuo to yield the product as a solid (83%, 2.48 g). ¹H NMR (500 MHz, DMSO-d₆) δ7.91 (dt, J=6.7, 3.3 Hz, 1H), 7.55 (s, 1H), 7.35 (dq, J=6.8, 3.6 Hz, 2H), 6.98 (dd, J=6.1, 3.2 Hz, 1H), 6.80 (s, 1H), 4.99 (s, 2H).

2-chloro-N,N-bis(propan-2-yl)acetamide. A 25.0 mL round bottom flask was charged with 1H-naphtho[2,3-d]imidazole-2-thiol (0.600 g, 3.0 mmol), dimethylformamide (6.00 mL) and 2-chloro-N,N-bis(propan-2-yl)acetamide (0.886 g, 5.0 mmol). Triethylamine (0.808 g, 8.00 mmol) was added dropwise under an argon atmosphere and the mixture stirred for 19 hours then treated with water (10.0 mL), partitioned with ethyl acetate, the organic layers combined, washed twice with water, twice with brine and concentrated in vacuo to yield the crude product as a solid. The crude material was purified by Reverse-phase Medium Pressure Liquid Chromatography (50 g C18 column, 80 -100% methanol in 25.0 mmolar ammonium formate) to provide a solid. The solid was then dissolved in ethyl acetate and washed with sodium bicarbonate and brine. The organic layer was dried over sodium sulfate, filtered, and concentrated in vacuo to yield the final product as a solid (0.175 g, 10%). ¹H NMR (500 MHz, DMSO-d₆) δ 12.57 (s, 1H), 7.97 (s, 1H), 7.93 (d, J=8.7 Hz, 2H), 7.80 (s, 1H), 7.34 (ddt, J=7.7, 5.5, 3.3 Hz, 2H), 4.52 (s, 2H), 4.12 (p, J=6.6 Hz, 1H), 3.55-3.48 (m, 1H), 1.29 (d, J=6.7 Hz, 6H), 1.22 (d, J=6.6 Hz, 6H). APCI [M+H] calc'd=342.1634 observed=342.1649.

### Example 12

### 2-[(5,6-dichloro-1H-1,3-benzodiazol-2-yl)sulfanyl]-N,N-bis(propan-2-yl)acetamide

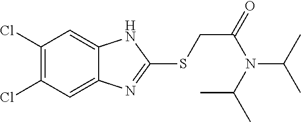 [SMILES: CC(C)N(C(=O)CSc1nc2cc(Cl)c(Cl)cc2[nH]1)C(C)C]

5,6-dichloro-1H-1,3-benzodiazole-2-thiol. A 100 mL round bottom flask was charged with 4,5-dichlorobenzene-1,2-diamine (5.00 g, 28.2 mrnol), ethanol (40.0 mL), water (3.00 mL) and sodium hydroxide (1.39 g, 35.0 mmol). Carbon disulfide (2.66 g, 35.0 mmol) was then added, in dropwise fahion, and the mixture heated to 75° C. for 3 hours. The reaction was quenched by adding water (40.0 mL) and then cooled to room temperature. The product was precipitated by adding 20.0 mL of a 1:9 acetic acid:water mixture. The solid was filtered, washed with hexanes, and dried in vacuo to yield the product as a solid (56%, 3.47 g), ¹H NMR (500 MHz, DMSO-d₆) δ 7.40-7.29 (m, 2H).

2-[(5,6-dichloro-1H-1,3-benzodiazol-2-yl)sulfanyl]-N,N-bis(propan-2-yl)acetamide. A 50.0 mL 14/20 round bottom flask was charged with 5,6-dichloro-1H-1,3-benzodiazole-2-thiol (0.613 g, 2.80 mmol), dimethylformamide (6.00 mL), and 2-chloro-N,N-bis(propan-2-yl)acetarnde (0.634 g, 3.50 mmol). Triethylamine (0.835 mL, 6.00 mmol) was added under an argon atmosphere and stirred at room temperature for 22 hours. The reaction was quenched by adding water (10.0 mL) and the crude product partitioned with ethyl acetate. The organic layer was washed six times with brine and concentrated in vacuo to provide a solid. The material was purified by Reverse-phase Medium Pressure Liquid Chromatography (50 g C18 column, 50-100% methanol in 25.0 mmolar ammonium formate). The formate salt was dissolved in ethyl acetate, washed with sodium bicarbonate, brine, dried over sodium sulfate, filtered, and concentrated in vacuo to yield the product as a solid (0.883 g, 86%). ¹H NMR (500 MHz, DMSO-d₆) δ 12.87 (s, 1H), 7.74 (s, 1H), 7.63 (s, 1H), 4.45 (s, 2H), 4.10-4.00 (m, 1H), 3.55-3.48 (m, 1H), 1.31-1.24 (m, 6H), 1.20 (d, J=6.6 Hz, 6H). APCI [M+H] calc'd=360.0698 observed=360.0712.

### Example 13

### N,N-bis(propan-2-yl)-2-[(1,5,6-trimethyl-1H-1,3-benzodiazol-2-yl)sulfanyl]acetamide

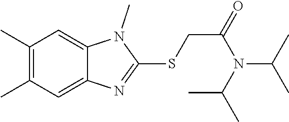 [SMILES: Cc1cc2nc(SCC(=O)N(C(C)C)C(C)C)n(C)c2cc1C]

1,5,6-trimethyl-1H-1,3-benzodiazole-2-thiol. A 50.0 mL 24/40 round bottom flask was charged with N-1,4,5-trimethylbenzene-1,2-diamine (1.47 g, 9.80 mmol, prepared according to the method described in Jhulki, I.; Chanani, P. K.; Abdeiwahed, S. H.; Be&y, T. P.; J. Am, Chem. Soc., 2016, 138, 8324), ethanol (10.0 mL), water (1.00 mL), sodium hydroxide (0.479 g, 12.0 mmol) and carbon disulfide (0.913 g, 12.0 mmol). The mixture was heated to 75° C. for 3 hours, quenched with water (12.0 mL) and cooled to room temperature. The product was precipitated by adding 9.00 mL of a 1:1 acetic acid:water mixture. The solid was filtered, washed with hexanes, and dried in vacuo to yield the product as a solid (1.55 g, 87%). ¹H NMR (500 MHz, DMSO-d₆) δ 12.53 (s, 1H), 7.14 (s, 1H), 6.96 (s, 1H), 158 (5, 3H), 2.26 (s, 3H), 2.24 (s, 3H).

N,N-bis(propan-2-yl)-2-[(1,5,6-trimethyl-1H-1,3-benzodiazo1-2-yl)sulfanyl]acetamide. A 50.0 mL 24/40 round bottom flask was charged with 1,5,6-trimethyl-1,3-benzodiazole-2-thiol (0.384 g, 2.0 mmol), dimethylformamide (4.00 mL) and 2-chloro-N,N-bis(propan-2-yl)acetamide (0.532 g, 3.00 mmol). Triethylamine (0.505 g, 5.0 mmol) was added under an argon atmosphere and the mixture stirred at room temperature for 22 hours. The reaction was quenched by adding water (10.0 mL) and the mixture partitioned with ethyl acetate. The organic layer was washed four times with brine, dried over sodium sulfate, filtered, and concentrated in vacuo to yield an oil. The crude material was purified by Reverse-phase Medium Pressure Liquid Chromatography (50 g C18 column, 50-100% methanol in 25.0 mmolar ammonium formate) to give a formate salt. The salt was dissolved in ethyl acetate, washed with sodium bicarbonate and brine. The organic layer was then dried over sodium sulfate, filtered, and concentrated in vacuo to yield an oil (0.387 g, 55%), ¹H NMR (500 MHz, Chloroform-d) δ7.43 (s, 1H), 7.03 (s, 1H), 4.50 (s, 2H), 4.19 (p, J=6.7 Hz, 1H), 3.69 (s, 3H), 3.52-3.45 (m, 1H), 2.37 (d, J=11.7 Hz, 6H), 1.42 (d, J=6.8 Hz, 6H), 1.25 (d, J=6.7 Hz, 6H). APCI [M+H] calc'd=334.1947 observed=334.1972.

### Example 14

### N,N-bis(propan-2-yl)-2-[(5,6,7-trifluoro-1H-1,3-benzodiazol-2-yl)sulfanyl]acetamide

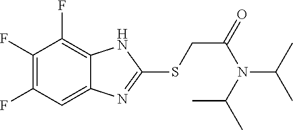 [SMILES: CC(C)N(C(=O)CSc1nc2cc(F)c(F)c(F)c2[nH]1)C(C)C]

3,4,5-trifluorobenzene-1,2-diamine. This compound was prepared according to the literature (J. Heterocyclic Chem., 2009, 46, 936.) by dissolving 2,3,4-trifluoro-6-nitroaniiine (2.00 g, 10.4 mmol) in anhydrous tetrahydrofuran (20.0 mL) and treating with 10% palladium on carbon (0.157 g, 1.47 mmol) for 20 hours under a hydrogen atmosphere (1 atm.). The crude reaction mixture was filtered through Celite and concentrated in vacuo to yield a solid (1.59 g, 99%). ¹H NMR (500 MHz, CDCl₃) δ6.33 (ddd, J=11.4, 6.9, 2.2 Hz, 1H), 3.31 (s, 4H). ¹⁹F NMR (470 MHz, CDCl₃) δ −147.55-−147.74 (m), −147.74-−147.96 (m), −154,28 (d, J=2.4 Hz), −154.32 (d, J=2.4 Hz), −172.65 (d, J=6.7 Hz), −172.72 (dd, J=21.8, 7.0 Hz).

5,6,7-trifluoro-1H-1,3-benzodiazole-2-thiol. The product was prepared using the method described by Kotovskaya, S. K.; Perova, N. M.; Baskakova, Z. M.; Rornanova, S. A.; Charushin, V. N.; Chupakhin, O. N.; Russ. J. Org. Chem., 2001, 37, 564. A 50.0 mL 24/40 round bottom flask was charged with 3,4,5-trifluorobenzene-1,2-diamine (1.48 g, 9.10 mmol), ethanol (18.0 mL), triethylamine (1.84 g, 18.2 mmol) and carbon disulfide (2.07 g, 27.3 mmol). The mixture was heated to 75° C. for 4 hours (argon atmosphere) and concentrated to a brown oil. The crude material was dissolved in ethanol (500 mL) and precipitated by adding water (150 mL). The solid was filtered, washed with hexanes, and dried n vacuo to yield the product as a solid (0.381 g, 21%). ¹H NMR (500 MHz, DMSO-d₆) δ7.07 (ddd, J=9.7, 6.0, 1.7 Hz, 1H). ¹⁹F NMR (470 MHz, DMSO-d₆) δ −142.94-−143.88 (m), −152.35-−153.24 (m), −170.21 (td, J=21.5, 6.8 Hz).

N,N-bis(propan-2-yl)-2-[(5,6,7-trifluoro-1H-1,3-benzodiazol-2-yl)sulfanyl]acetamide. A 50.0 mL 14/20 round bottom flask was charged with 5,6,7-trifluoro-1H-1,3-benzodiazole-2-thiol (0.350 g, 1.70 mmol), dimethylformamide (5.00 mL) and 2-chloro-N,N-bis(propan-2-yl)acetamide (0.532 g, 3.00 mmol). Triethylamine (0.506 g, 5.00 mmol) was added under an argon atmosphere and the mixture stirred at room temperature for 21 hours. The reaction was quenched by adding water (10.0 mL) and the crude material partitioned with ethyl acetate. The organic layer was washed four times with brine and concentrated in vacuo. The crude material was purified by Reverse-phase Medium Pressure Liquid Chromatography (50 g C18 column, 70-100% methanol in 25.0 mmolar ammonium formate) to give a formate salt. The salt was dissolved in ethyl acetate and washed with sodium bicarbonate and brine. The organic layer was dried over sodium sulfate, filtered, and concentrated in vacuo to yield the final product as a solid (0.303 g, 80%). ¹H NMR (500 MHz, DMSO-d₆) δ 13.22 (d, J=153.4 Hz, 1H), 7.69-7.24 (m, 1H), 4.60-4.34 (m, 2H), 4.15-3.86 (m, 1H), 3.61-3.40 (m, 1H), 1.58-0.91 (m, 12H), ¹⁹F NMR (470 MHz, DMSO-d₆) δ −141.53-−145.48 (m, 1F), −151.39-−153.64 (m, 1F), −168.86-−-172.22 (rn, 1F), APCI [M+H] calc'd=346.1195, observed=346,1209.

### Example 15

### 2-[(6-phenoxy-1H-1,3-benzodiazol-2-yl)sulfanyl]-N,N-bis(propan-2-yl)acetamide

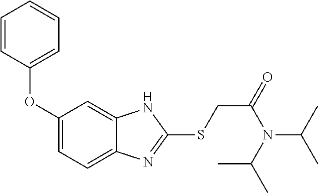 [SMILES: CC(C)N(C(=O)CSc1nc2ccc(Oc3ccccc3)cc2[nH]1)C(C)C]

6-phenoxy-1H-1,3-benzothazole-2-thiol. A 50.0 mL 24/40 round bottom flask was charged with 4-phenoxybenzene-1,2-diamine (0.500 g, 2.50 mmol), ethanol (10.0 mL), water (2.00 mL), sodium hydroxide (0.139 g, 3.50 mmol), and carbon disulfide (0.211 mL, 3.50 mmol). The mixture was heated to 75° C. for 3 hours, quenched with water (5.00 mL) and then cooled to room temperature. The product was precipitated by adding 6 mL of a 1:1 acetic acid:water mixture. The solid was filtered, washed with hexanes, and dried in vacuo to yield a solid (0.511g, 84%). ¹H NMR (500 MHz, DMSO-d₆) δ 12.51 (s, 2H), 7.41-7.29 (m, 2H), 7.17-7.08 (m, 2H), 7.02-6.93 (m, 2H), 6.83 (dd, J=8.6, 2.3 Hz, 1H), 6.71 (d, J=2.3 Hz, 1H).

2-[(6-phenoxy-1H-1,3-benzodiazol-2-yl)sulfanyl]-N,N-bis(propan-2-yl)acetamide. A 50.0 mL round bottom flask was charged with 6-phenoxy-1H-1,3-benzodiazole-2-thiol (0.400 g, 1.60 mmol), dimethylformamide (2.50 mL) and 2-chloro-N,N-bis(propan-2-yl)acetarnde (0.355 g, 2.00 mmol). Triethylamine (0.303 g, 3.00 mmol) was added under an argon atmosphere and the reaction mixture stirred at room temperature for 17 hours. The reaction was quenched by adding water (30.0 mL) and then partitioned into ethyl acetate. The organic layer was washed four times with brine and concentrated in vacuo to yield an oil. The crude material was purified by Reverse-phase Medium Pressure Liquid Chromatography (50 g C18 column, 70-100% methanol in 25.0 mmolar ammonium formate) to provide a formate salt. The product was dissolved in ethyl acetate then washed with sodium bicarbonate and brine. The organic layer was dried over sodium sulfate, filtered, and concentrated in vacuo to yield the product as a solid (0.244 g, 41%). ¹H NMR (500 MHz, DMSO-d₆) δ 12.56 (d, J=42.3 Hz, 1H), 7.57-7.25 (m, 3H), 7.18-6.80 (m, 4H), 4.41 (d, J=6.3 Hz, 2H), 4.14-3.99 (m, 1H), 3.59-3.44 (m, 1H), 1.29 (d, J=6.7 Hz, 6H), 1.19 (dd, J=6.1, 2.0 Hz, 6H). APCI [M+H] calc'd=384.1740 observed=384.1750.

### Example 16

### 2-[(6-methoxy-1H-1,3-benzodiazol-2-yl)sulfanyl]-N,N-bis(propan-2-yl) acetamide

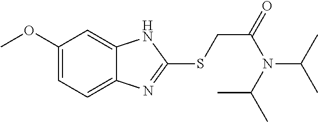 [SMILES: COc1ccc2nc(SCC(=O)N(C(C)C)C(C)C)[nH]c2c1]

A 50.0 mL round bottom flask was charged with 6-methoxy-1H-1,3-benzodiazole-2-thiol (0.270 g, 1.50 mmol.), dimethylformamide (5.00 mL), and 2-chloro-N,N-bis(propan-2-yl)acetamide (0.355 g, 2.00 mmol). Triethylamine was added (0.303 g, 3.00 mmol) under an argon atmosophere and the reaction stirred at room temperature for 17 hours. The reaction was quenched by adding water (30.0 mL) and the crude material partitioned into ethyl acetate. The organic layer was washed four times with brine and concentrated in vacuo to yield an oil. The crude material was purified by Reverse-phase Medium Pressure Liquid Chromatography (50 g C18 column, 70-100% methanol in 25.0 mmolar ammonium formate) to provide a formate salt. The product was dissolved in ethyl acetate and washed with sodium bicarbonate and brine. The organic layer was dried over sodium sulfate, filtered, and concentrated in vacuo to yield a solid (0.385 g, 84%). ¹H NMR (500 MHz, DMSO-d₆) δ 12.36 (s, 1H), 7.45-6,62 (m, 3H), 4.45-4.29 (m, 2H), 4.15-3.95 (m, 1H), 3.74 (s, 3H), 3.49 (t, J=6.9 Hz, 1H), 1.28 (d, J=6.7 Hz, 6H), 1.18 (d, J=6.5 Hz, 6H). APCI [M+H] calc'd=322.1583 observed=322.1529.

### Example 17

### N,N-bis(propan-2-yl)-2-{[6-(propan-2-yl)-1H-1,3-benzodiazol-2-yl]sulfanyl}acetamide

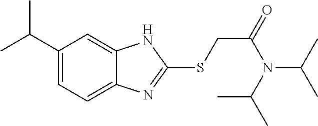 [SMILES: CC(C)c1ccc2nc(SCC(=O)N(C(C)C)C(C)C)[nH]c2c1]

6-(propan-2-yl)-1H-1,3-benzodiazole-2-thiol. To a 50.0 mL 24/40 round bottom flask was added 4-(propah-2-yl)benzehe-1,2-diamine (1.55 g, 11.0 mmol, prepared according to the method described by Steven John Woodhead, Martyn Frederickson, Christopher Hamlett, Andrew James Woodhead, Marinus Leendert Verdonk, Hannah Fiona Sore, David Winter Walker, Peter Biurton, an Collins, Kwai Ming Cheung, John Caldwell, Tatiana Faria Da Fonseca Mchardy, Richard William Arthur Luke, Zbigniew Stanley Matusiak, Andrew Leach, Jeffrey James Morris, WO2008075109A1), ethanol (15.0 mL), water (2.00 mL), sodium hydroxide (0.519 g, 13.0 mmol) and carbon disulfide (0.988 g, 13.0 mmol). The mixture was heated to 75° C. for 3 hours and then quenched by adding water (40.0 mL) and cooling to room temperature. The product was precipitated by adding 20.0 mL of a 1:1 acetic acid:water mixture and the solid filtered, washed with hexanes, and dried in vacuo to yield the product as powder (0.864 g, 40%), ¹H NMR (500 MHz, DMSO-d₆) δ 12.42 (d, 9.0 Hz, 2H), 7.10-6.88 (m, 3H), 2.91 (p, J=6.8 Hz, 1H), 1.18 (dd, J=7.0, 1.7 Hz, 6H).

N,N-bis(propan-2-yl)-2-{[6-(propan-2-yl)-1H-1,3-benzodiazol-2-yl]sulfanyl}acetamide

A 50.0 mL round bottom flask was charged with 6-(propan-2-yl)-1H-1,3-benzodiazole-2-thiol. (0.576 g, 3.00 mmol), dimethylformamide (5.00 mL), and 2-chloro-N,N-bis(propan-2-yl)acetamide (0.680 g, 3.80 mmol). The flask was sealed (argon atmosphere) and triethylamine (0.500 g, 5.00 mmol) added. The reaction was stirred at room temperature for 20 hours and quenched by with water (30.0 mL). The crude material was partitioned between water and ethyl acetate and the organic layer washed four times with brine, dried over sodium sulfate, and concentrated in vacuo to yield an oil. The crude oil was purified by Reverse-phase Medium Pressure Liquid Chromatography (50 g C18 column, 70-100% methanol in 25.0 mmolar ammonium formate) to provide a formate salt. The product was dissolved in ethyl acetate, washed with sodium bicarbonate and brine. The organic layer was dried over sodium sulfate, filtered, and concentrated in vacuo to yield the final product as a solid (0.436 g, 46%). ¹H NMR (500 MHz, DMSO-d₆) δ 12.38 (s, 1H), 7.48-6.88 (m, 4H), 4,39 (d, J=3.1 Hz, 2H), 4.06 (tt, J=15.2, 7.0 Hz, 1H), 3.50 (s, 1H), 2.96 (hept, J=7.0 Hz, 1H), 1.43-1.10 (m, 16H). APCI [M+H] calc'd=334.1947, observed=334.2001.

### Example 18

### 2-[(5,6-dichloro-1H-1,3-benzodiazol-2-yl)sulfonyl]-N,N-bis(propan-2-yl)acetamide

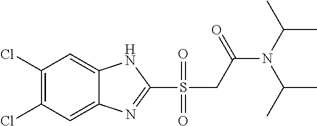 [SMILES: CC(C)N(C(=O)CS(=O)(=O)c1nc2cc(Cl)c(Cl)cc2[nH]1)C(C)C]

A 25.0 mL 14/20 round bottom flask was charged with 2-[(5,6-dichloro-1H-1,3-benzodiazol-2-yl)sulfanyl]-N,N-bis(oropan-2-yl)acetamide (0.120 g, 0.300 mmol) and dichloromethane (5.00 mL) and cooled to 0° C. in an ice bath. Meta-chloroperoxybenzoic acid (0.206 g, 1.20 mmol) was added. After 5 minutes a precipitate formed and additional dichloromethane (5.00 mL) was added. After an additional 5 minutes the reaction was quenched with the addition of saturated aqueous sodium carbonate (10.0 mL) and partitioned. The organic layer was dried over sodium sulfate, filtered, and concentrated in vacuo. The crude material was purified by Reverse-phase Medium Pressure Liquid Chromatography (50 g C18 column, 70-100% methanol in 25.0 rnmolar ammonium formate) to provide a formate salt. The product was dissolved in ethyl acetate, washed with sodium bicarbonate and brine. The organic layer was dried over sodium sulfate, filtered, and concentrated in vacuo to yield a powder (36%, 0.043 g). ¹H NMR (500 MHz, DMSO-d₆) δ7.67 (s, 2H), 4.52 (s, 2H), 4.17 (p, J=6.6 Hz, 1H), 3.45 (dd, J=11.9, 5.2 Hz, 1H), 1.27 (d, J=6.7 Hz, 6H), 1.16-1.08 (m, 6H). APCI [M−H] calc'd=390.0451 observed=390.0444.

### Example 19

### 3-(5,6-dichloro-1H-1,3-benzodiazol-2-yl)-N,N-bis(propan-2-yl)propenamide

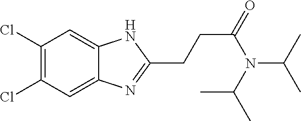 [SMILES: CC(C)N(C(=O)CCc1nc2cc(Cl)c(Cl)cc2[nH]1)C(C)C]

3-[bis(propan-2-yl)carbamoyl]propanoic acid. A 100 mL 24/40 round bottom flask was charged with succinic anhydride (2.00 g, 19.0 mmol) and ethyl acetate (20.0 mL) and treated dropwise with diisopropylamine (3.08 mL, 22.0 mmol). The mixture was heated to reflux for 23 hours, then concentrated in vacuo to yield the product as an oil (3.8 g, 100%). ¹H NMR (500 MHz, DMSO-d₅) δ4.00 (dq, J=13.2, 6.9 Hz, 1H), 3.18 (p, J=6.4 Hz, 1H), 2.45 (dd, J=7.2, 6.1 Hz, 2H), 2.37-2.30 (m, 2H), 1.24 (d, J=6.7 Hz, 4H), 1.17-1.08 (m, 8H).

3-(5,6-dichloro-1H-1,3-benzodiazol-2-yl)-N,N-bis(propan-2-yl)propenamide, A 100 mL 24/40 round bottom flask was charged with 4,5-dichlorobenzene-1,2-diamine (0.299 g, 2.20 mmol) and anhydrous 1,4-dioxane (6.00 mL). 3-[bis(propan-2-yl)carbamoyl]propanoic acid (0.482 g, 2.40 mmol) was added, followed by polyphosphoric acid (1.63 g, 16.0 mmol). The reaction vessel was heated to 105° C. and stirred under an argon atmosphere for 20 hours. The reaction was quenched by adding water (30.0 mL) and sodium carbonate (12.3 g, 117 mmol). The reaction mixture was partitioned with ethyl acetate and concentrated in vacuo. The crude material was purified by Reverse-phase Medium Pressure Liquid Chromatography (50 g C18 column, methanol in 25.0 mmolar ammonium formate). The resulting formate salt was then partitioned between ethyl acetate and a saturated sodium bicarbonate solution, the organic layer washed with brine, dried over sodium sulfate, filtered, and concentrated in vacuo to yield pure product as a powder (0.151 g, 22%). ¹H NMR (500 MHz, DMSO-d₆) δ 11.88 (s, 1H), 7.20 (s, 2H), 4.11-3.95 (m, 1H), 3.46 (s, 1H), 2.94 (dd, J=9.0, 6.3 Hz, 2H), 2.78 (dd, J=9.0, 6.3 Hz, 2H), 2.26 (s, 6H), 1.27 (d, J=6,7 Hz, 6H), 1.18-1.10 (m, 6H). APCI [M+H] calc'd=302.2226 observed=302.2274, m.p.=182° C.

### Example 20

### 3-(6-chloro-1H-1,3-benzodiazol-2-yl)-N,N-bis(propan-2-yl)propenamide

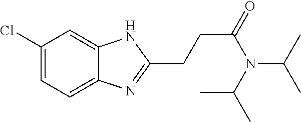 [SMILES: CC(C)N(C(=O)CCc1nc2ccc(Cl)cc2[nH]1)C(C)C]

A 100 mL round bottom flask was charged with 4-chlorobenzene-1,2-diamine (0.570 g, 4.00 mmol), anhydrous 1,4-dioxane (25.0 mL), 3-[bis(propan-2-yl)carbamoyl]propanoic acid (1.01 g, 5.00 mmol) and polyphosphoric acid (3.26 g, 32.0 mmol). The reaction mixture was heated to 105° C. under an argon atmosphere with stirring for 20 hours. The mixture was quenched with water (30.0 mL) and sodium carbonate (12.3 g, 117 mmol) and then partitioned with ethyl acetate. The organic layer was concentrated, and the crude material purified by Reverse-phase Medium Pressure Liquid Chromatography (50 g C18 column, methanol in 25.0 mmoiar ammonium formate). The product was partitioned between ethyl acetate and saturated sodium bicarbonate, washed with brine, dried over sodium sulfate, filtered, and concentrated to yield the freebase of the pure product as a powder (0.560 g, 46%). ¹H NMR (500 MHz, DMSO-d₆) δ 12.56-12.25 (m, 1H), 7.63-7.37 (m, 2H), 7.11 (ddd, J=12.9, 8.4, 2.0 Hz, 1H), 4.02 (tt, J=11.1, 6.9 Hz, 1H), 3.45 (br s, 1H), 2.98 (dd, J=8.7, 6.4 Hz, 2H), 2.80 (dd, J=8.7, 6.4 Hz, 2H), 1.25 (d, J=6.7 Hz, 6H), 1.12 (d, J=6.6 Hz, 6H). APCI [M+H] calc'd=308.1524 observed=308.1550.

### Example 2

### 3-(5,7-dimethyl-1H-1,3-benzodiazol-2-yl)-N,N-bis(propan-2-yl)propenamide

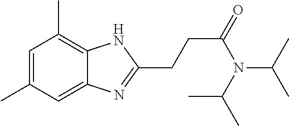 [SMILES: Cc1cc(C)c2[nH]c(CCC(=O)N(C(C)C)C(C)C)nc2c1]

A 100 mL 24/40 round bottom flask was charged with 3,5-dimethylbenzene-1,2-diamine (0.484 g, 3.50 mmol), anhydrous 1,4-dioxane (20.0 mL), 3-[bis(propan-2-yl)carbamoyl]propanoic add (1.01 g, 5.00 mmol) and polyphosphoric add (2.49 g, 25.6 mmol). The reaction mixture was heated to 105° C. under argon with stirring for 20 hours then quenched by adding water (30.0 mL) and sodium carbonate (12.3 g, 117 mmol). The mixture was partitioned into ethyl acetate and the organic layer concentrated and purified by Reverse-phase Medium Pressure Liquid Chromatography (50 g C18 column, methanol in 25.0 mmolar ammonium formate). The product was partitioned between ethyl acetate and a saturated sodium bicarbonate solution. The organic layer washed with brine, dried over sodium sulfate, filtered, and concentrated to yield a foam, This material was purified by filtration through a plug of silica gel (eluted with 10% methanol in dichloromethane). This material was concentrated in vacuo and recrystallized from dichloromethane and hexanes to yield the final product as a powder (0.344 g, 33%). ¹H NMR (500 MHz, DMSO-d₆) δ 11.92 (d, J=26.3 Hz, 1H), 7.00 (s, 1H), 6.71 (s, 1H), 4.05 (p, J=6.6 Hz, 1H), 3.46 (br s, 1H), 2.95 (dd, J=9.0, 6.3 Hz, 2H), 2.78 (t, J=7.6 Hz, 2H), 2.40 (s, 3H), 2.32 (s, 3H), 1.31-1.20 (m, 6H), 1.13 (d, J=6.6 Hz, 6H). APCI [M+H] calc'd=302.2226 observed=302.2258.

### Example 21A

### 3-(5,6-dimethyl-1H-1,3-benzodiazol-2-yl)-N,N-bis(propan-2-yl)propenamide

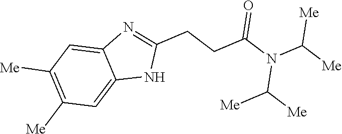 [SMILES: Cc1cc2nc(CCC(=O)N(C(C)C)C(C)C)[nH]c2cc1C]

A 100 mL 24/40 round bottom flask was charged with 4,5-dimethylbenzene-1,2-diamine (0.299 g, 2.20 mmol), anhydrous 1,4-dioxane (6.00 mL), 3-[bis(propan-2-yl)carbarnoyl]propanoic acid (0.482 g, 2.39 mmol) and polyphosphoric acid (1.6.3 g, 16.0 mmol). The reaction mixture was heated to 105° C. under argon with stirring for 20 hours then quenched by adding water (30.0 mL) and sodium carbonate (12.3 g, 117 mmol). The mixture was partitioned into ethyl acetate and the organic layer concentrated and purified by Reverse-phase Medium Pressure Liquid Chromatography (50 g C18 column, 0 to 70% methanol in 25.0 mmolar aqueous ammonium formate). The product was partitioned between ethyl acetate and a saturated sodium bicarbonate solution. The organic layer washed with brine, dried over sodium sulfate, filtered, and concentrated to yield a white solid (0.151 g, 22%). ¹H NMR (500 MHz, DMSO-d₆) δ 11.88 (s, 1H), 7.20 (s, 2H), 4.11-3.95 (m, 1H), 3.46 (s, 1H), 2.94 (dd, J=9.0, 6.3 Hz, 2H), 2.78 (dd, J=9.0, 6.3 Hz, 2H), 2.26 (s, 6H), 1.27 (d, J=6.7 Hz, 6H), 1.18-1.10 (m, 6H). APCI [M+H] calc'd=302.2226 observed=302.2274.

### Example 22

### 3-(5,7-dichloro-1H-1,3-benzodiazol-2-yl)-N,N-bis(propan-2-yl)propenamide

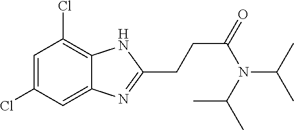 [SMILES: CC(C)N(C(=O)CCc1nc2cc(Cl)cc(Cl)c2[nH]1)C(C)C]

A 100 mL round bottom flask was charged with 3,5-dichlorobenzene-1,2-diamine (0.354 g, 2.00 mmol), anhydrous 1,4-dioxane (10.0 mL), 3-[bis(pr.pan-2-yl)carbamoyl]propanoic acid (0.601 g, 3.00 mmol) and polyphosphoric acid (1.56 g, 15.9 mmol). The reaction mixture was heated to 105° C. under argon with stirring for 20 hours. The reaction was then quenched with water (30.0 mL) and sodium carbonate (12.3 g, 117 mmol) and partitioned into ethyl acetate, The organic layer was concentrated in vacuo and the crude material purified by Reverse-phase Medium Pressure Liquid Chromatography (50 g C18 column, methanol in 25.0 mmolar ammonium formate). The formate salt was partitioned between ethyl acetate and saturated sodium bicarbonate, the organic layer washed with brine, dried over sodium sulfate, filtered, and concentrated to the product as a solid (0.235 g, 34%). ¹H NMR (500 MHz, DMSO-d₆) δ 12.72 (d, J=95.0 Hz, 1H), 7.67-7.40 (m, 1H), 7.37-7.16 (m, 1H), 4.05 (tt, J=13.4, 6.0 Hz, 1H), 3.45 (s, 1H), 3.00 (dd, J=8.3, 6.5 Hz, 2H), 2.82 (q, J=7.4 Hz, 2H), 1.24 (d, J=6.7 Hz, 6H), 1.13 (d, J=6.6 Hz, 6H). APCI [M+H] calc'd=342.1134 observed=342.1145.

### Example 23

### N,N-bis(propan-2-yl)-3-(5,6,7-trifluoro-1H-1,3-benzodiazol-2-yl)propenamide

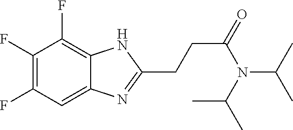 [SMILES: CC(C)N(C(=O)CCc1nc2cc(F)c(F)c(F)c2[nH]1)C(C)C]

A 100 mL round bottom flask was charged with 3,4,5-trifluorobenzene-1,2-diamine (0.513 g, 3.20 mmol), anhydrous 1,4-dioxane (15.0 mL), 3-[bis(propan-2-yl)carbamoyl]propanoic acid (1.01 g, 5.00 mmol) and polyphosphoric acid (2.49 g, 25.6 mmol). The reaction mixture was heated to 105° C. under an argon atmosphere with stirring for 20 hours. The reaction was quenched with water (30.0 mL) and sodium carbonate (12.3 g, 117 mmol) and partitioned into ethyl acetate. The crude material was purified by Reverse-phase Medium Pressure Liquid Chromatography (50 g C18 column, methanol in 25.0 mmolar ammonium formate). The product was partitioned between ethyl acetate and a saturated sodium bicarbonate solution. The organic layer washed with brine, dried over sodium sulfate, and concentrated in vacuo to yield the product. This material was recrystallized from dichloromethane and hexanes to provide the final product as a powder (0.278 g, 27%). ¹H NMR (500 MHz, DMSO-d₆) δ 12.75 (s, 1H), 7.40 (ddd, J=10.3, 6.1, 1.7 Hz, 1H), 4.04 (p, J=6.7 Hz, 1H), 3.46 (br s, 1H), 2.98 (dd, J=8.5, 6,4 Hz, 2H), 2.81 (dd, J=8.6, 6.4 Hz, 2H), 1.24 (d, J=6.7 Hz, 6H), 1.13 (d, J=6.6 Hz, 6H). ¹⁹F NMR (470 MHz, DMSO-d₆) δ −143.32, −152.23, −171.93. APCI [M+H] calc'd=328.1631 observed=328.1642.

### Example 24

### N,N-bis(propan-2-yl)-3-(5,6,7-trichloro-1H-1,3-benzodiazol-2-yl)propenamide

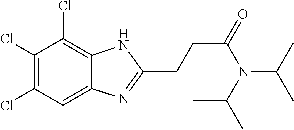 [SMILES: CC(C)N(C(=O)CCc1nc2cc(Cl)c(Cl)c(Cl)c2[nH]1)C(C)C]

A 100 mL 24/40 round bottom flask was charged with 3,4,5-trichlorobenzene-1,2-diamine (0.629 g, 3.0 mmol, prepared according to the method described in Gudmundsson, K. S.; Tidwell, J.; Lippa, N.; Koszalka, G. W.; van Draanen N.; Ptak, R. G.; Drach, J, C.; Townsend, L. B.; J. Med. Chem., 2000, 43, 2464.), anhydrous 1,4-dioxane (15.0 mL), 3-[bis(propan-2-yl)carbamoyl]propanoic acid (0.804 g, 4.00 mmol) and polyphosphoric acid (2.35 g, 24.0 mmol). The reaction mixture was heated to 105° C. with stirring under an argon atmosphere for 20 hours. The reaction mixture was quenched with water (30.0 mL) and sodium carbonate (117 mmol, 12.3 g) and partitioned into ethyl acetate. The organic layer was concentrated in vacuo purified by Reverse-phase Medium Pressure Liquid Chromatography (50 g C18 column, methanol in 25.0 mmolar ammonium formate). The salt was partitioned between ethyl acetate and saturated sodium bicarbonate, the organic layer washed with brine, dried over sodium sulfate, filtered, and concentrated to yield a solid. This material was recrystallized from dichloromethane (with 3% methanol) and hexanes to yield the final product as a powder (0.678 g, 60%). ¹H NMR (500 MHz, DMSO-d₆) δ 12.83 (s, 1H), 7.75 (s, 1H), 4.05 (dq, J=20.3, 7.1, 6.2 Hz, 1H), 3.45 (s, 1H), 3.02 (t, J=7.4 Hz, 2H), 2.82 (t, J=7.4 Hz, 2H), 1.24 (d, J=6.8 Hz, 6H), 1.13 (d, J=6.6 Hz, 6H). APCI [M+H] calc'd=376.0744 observed=376.0759.

### Example 25

### N,N-bis(propan-2-yl)-3-[6-(trifluoromethoxy)-1H-1,3-benzodiazol-2-yl]propenamide

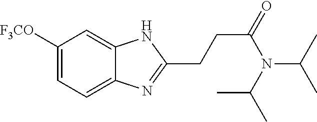 [SMILES: CC(C)N(C(=O)CCc1nc2ccc(OC(F)(F)F)cc2[nH]1)C(C)C]

A 100 mL 24/40 round bottom flask was charged with 4-(trifluoromethoxy)benzene-1,2-diamine (0.384 g, 2.00 mmol), anhydrous 1,4-dioxane (10.0 mL), 3-[bis(propan-2-yl)carbamoyl]propanoic acid (0.600 g, 3.00 mmol) and polyphosphoric acid (1.56 g, 16.0 mmol). The reaction mixture was heated to 105° C. under an argon atmosphere with stirring for 20 hours. The reaction was quenched with water (30.0 mL) and sodium carbonate (117 mmol, 12.3 g) and partitioned with ethyl acetate. The organic layer was concentrated and purified by Reverse-phase Medium Pressure Liquid Chromatography (50 g C18 column, methanol in 25.0 mmolar ammonium formate), Fractions containing product were combined and concentrated in vacuo. The product was partitioned between ethyl acetate and saturated sodium bicarbonate, the organic layer washed with brine, dried over sodium sulfate, filtered, and concentrated to yield a solid. This material was further purified by silica gel chromatography (0-5% methanol in dichloromethane) and recrystallized from dichloromethane and hexanes to yield the final product as a solid (0.284 g, 41%). ¹H NMR (500 MHz, DMSO-d₆) δ 12.42 (d, J=17.2 Hz, 1H), 7.68-7.33 (m, 2H), 7.26-6.98 (m, 1H), 4.04 (p, J=6.7 Hz, 1H), 3.39 (s, 1H), 3.00 (dd, J=8.7, 6.4 Hz, 2H), 2.82 (t, J=7.6 Hz, 2H), 1.26 (d, J=6.7 Hz, 6H), 1.13 (d, J=6.6 Hz, 6H). ¹⁹F NMR (470 MHz, DMSO-d₆) δ −57.06 (d, J=21.7 Hz). APCI [M+H] calc'd=358.1736 observed=358.1758.

### Example 26

### N,N-bis(propan-2-yl)-3-[6-(trifluoromethyl)-1H-1,3-benzodiazol-2-yl]propenamide

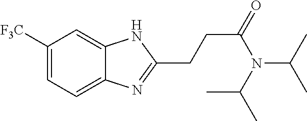 [SMILES: CC(C)N(C(=O)CCc1nc2ccc(C(F)(F)F)cc2[nH]1)C(C)C]

A 100 mL roundbottom flask was charged with 4-(trifluoromethyl)benzene-1,2-diamine (0.352 g, 2.00 mmol,), anhydrous 1,4-dioxane (10.0 mL), 3-[bis(propan-2-yl)carbamoyl]propanoic acid (0.600 g, 3.00 mmol) and polyphosphoric acid (1.56 g, 16.0 mmol). The reaction mixture was heated to 105° C. under an argon atmosphere with stirring for 20 hours. The reaction was quenched with water (30.0 mL) and sodium carbonate (12.3 g, 117 mmol) and partitioned into ethyl acetate. The organic layer was concentrated and purified by Reverse-phase Medium Pressure Liquid Chromatography (50 g C18 column, methanol in 25.0 mmolar ammonium formate). The salt was partitioned between ethyl acetate and sodium bicarbonate, brine, dried over sodium sulfate, filtered, and concentrated in vacuo. This material was further purified by silica gel chromatography (0-10% methanol in dichloromethane) and twice recrystallized from dichloromethane (containing 5% methanol) and hexanes to yield the final product as a powder (0.269 g, 39%). ¹H NMR (500 MHz, DMSO-d₆) δ 12.69 (d, J==94.9 Hz, 1H), 7.97-7.55 (m, 2H), 7.54-7.33 (m, 1H), 4.13-3.96 (m, 1H), 3.44 (s, 1H), 3.02 (d, J=8.9 Hz, 2H), 2.84 (t, J=7.5 Hz, 2H), 1.25 (d, J=6.7 Hz, 6H), 1.13 (d, J=6.7 Hz, 6H). ¹⁹F NMR (470 MHz, DMSO-d₆) δ −58.63 (d, J=25.6 Hz), APCI [M+HH] calc'd=342.1787 observed=342.1816.

### Example 27

### 3-(6-phenoxy-1,3-benzodiazol-2-yl)-N,N-bis(propan-2-yl)propenamide

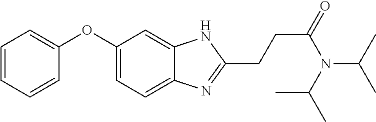 [SMILES: CC(C)N(C(=O)CCc1nc2ccc(Oc3ccccc3)cc2[nH]1)C(C)C]

A 100 mL roundbottom flask was charged with 4-phenoxybenzene-1,2-diamine (0.301 g, 1.50 mmol), anhydrous 1,4-dioxane (10.0 mL), 3-[bis(propan-2-yl)carbamoyl]propanoic acid (0.503 g, 2.50 mmol) and polyphosphoric acid (1.22 g, 12.5 mmol). The reaction mixture was heated to 105° C. under an argon atmosphere with stirring for 20 hours. The reaction was quenched with water (30.0 mL) and sodium carbonate (117 mmol, 12.3 g) and partitioned into ethyl acetate. The organic layer was concentrated and purified by Reverse-phase Medium Pressure Liquid Chromatography (50 g C18 column, methanol in 25.0 mmolar ammonium formate). The residue was partitioned between ethyl acetate and a saturated sodium bicarbonate solution and the organic layer washed with brine, dried over sodium sulfate, filtered, and concentrated to a solid, which was recrystallized from dichloromethane and hexanes to yield the final product powder (0.137 g, 25%). ¹H NMR (500 MHz, Chloroform-d) δ7.57-7.44 (m, 1H), 7.37-7.25 (m, 3H), 7.20 (d, J=5.5 Hz, 1H), 7.05 (dt, J=7.3, 1.2 Hz, 1H), 7.03-6.91 (m, 3H), 4.02 (p, J=6.7 Hz, 1H), 3.51 (s, 1H), 3.35-3.20 (m, 2H), 2.92-2.78 (m, 2H), 1.43 (d, J=6.8 Hz, 6H), 1.19 (d, J=6.6 Hz, 6H). APCI [M+H] calc'd=366.2176 observed=366.2187.

### Example 28

### 3-(5,7-difluoro-1H-1,3-benzodiazol-2-yl)-N,N-bis(propan-2-yl)propenamide

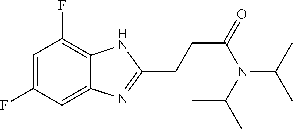 [SMILES: CC(C)N(C(=O)CCc1nc2cc(F)cc(F)c2[nH]1)C(C)C]

A 100 mL 24/40 round bottom flask was charged with 3,5-difluorobenzene-1,2-diamine (0.432 g, 3.00 mmol), anhydrous 1,4-dioxane (10.0 mL), 3-[bis(propan-2-yl)carbamoyl]propanoic acid (0.805 g, 4.00 mmol) polyphosphoric acid (2.35 g, 24.0 mmol). The reaction mixture was heated to 105° C. under an argon atmosphere with stirring for 20 hours. The reaction was quenched with water (30.0 mL) and sodium carbonate (117 mmol, 12.3 g), partitioned into ethyl acetate, and concentrated. The crude material was purified by Reverse-phase Medium Pressure Liquid Chromatography (50 g C18 column, methanol in 25.0 mmolar ammonium formate). The salt was partitioned between ethyl acetate and a saturated sodium bicarbonate solution, and the oraanic layer washed with brine, dried over sodium sulfate, filtered, and concentrated in vacuo. The product was additionally purified by silica gel chromatography (0-10% methanol in dichloromethane). This material was recrystallized from dichloromethane and hexanes to yield the final product as a powder (0.168 g, 18%). ¹H NMR (500 MHz, DMSO-d₆) δ 12.69 (m, 1H), 7.26-6.80 (m, 2H), 4.14-3.97 (m, 1H), 3.45 (br s, 1H), 2.97 (t, J=7.4 Hz, 2H), 2.81 (q, J=7,9, 7.3 Hz, 2H), 1.25 (d, J=6.7 Hz, 6H), 1.13 (d, J=6.6 Hz, 6H), ¹⁹F NMR (470 MHz, DMSO-d₆) δ −118.43 (d, J=9.9 Hz), −118,81 (t, J=9.1 Hz), −120,52 (t, J=9.9 Hz), −125.86 (d, J=11.5 Hz), −126.18 (d, J=10.6 Hz), −127.97 (d, J=10.7 Hz). APCI [M+H] calc'd=310.1725 observed=310.1743.

### Example 29

### 3-(5-chloro-1,3-benzoxazol-2-yl)-N,N-bis(propan-2-yl)propenamide

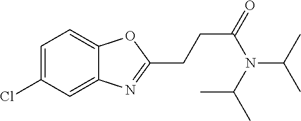 [SMILES: CC(C)N(C(=O)CCc1nc2cc(Cl)ccc2o1)C(C)C]

A 100 mL round bottom flask was charged with 2-amino-4-chlorophenol (0.430 g, 3.00 mmol), anhydrous 1,4-dioxane (15.0 mL), 3-[bis(propan-2-yl)carbamoyl]propanoic acid (0.804 g, 4.00 mmol) and polyphosphoric acid (2.35 g, 24.0 mmol). The reaction mixture was heated to 105°0 C. under an argon atmosphere with stirring for 20 hours. The reaction was quenched with water (30.0 mL) and sodium carbonate (12.3 g, 117 mmol) and partitioned into ethyl acetate. The organic layer was concentrated to yield the crude product which was purified by Reverse-phase Medium Pressure Liquid Chromatography (50 g C18 column, methanol in 25.0 mmolar ammonium formate). The formate salt was partitioned between ethyl acetate and saturated sodium bicarbonate, the organic layer washed with brine, dried over sodium sulfate, filtered, and concentrated to yield the product. This material was additionally purified by silica gel chromatography (0-5% methanol in dichioromethane) and recrystallized twice from dichioromethane and hexanes to yield the final product as yellow powder (0.100 g, 10%). ¹H NMR (500 MHz, DMSO-d₆) δ 7.76 (d, J=2.1 Hz, 1H), 7.69 (d, J=8,6 Hz, 1H), 7.37 (dd, J=8.6, 2.1 Hz, 1H), 4.05 (p, J=6.7 Hz, 1H), 3.55 (s, 1H), 3.10 (t, J=7.1 Hz, 2H), 2.87 (t, J=7.1 Hz, 2H), 1.23 (d, J=6.7 Hz, 6H), 1.15 (d, J=6.6 Hz, 6H), APCI [M+H] calc'd=309.1364 observed=309.1382.

### Example 30

### 3-{5-chloro-1H-imidazo[4,5-b]pyridin-2-yl}-N,N-bis(propan-2-yl)propenamide

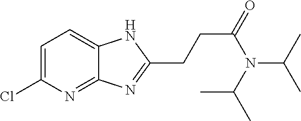 [SMILES: CC(C)N(C(=O)CCc1nc2nc(Cl)ccc2[nH]1)C(C)C]

A 100 mL 24/40 round bottom flask was charged with 6-chloropyridine-2,3-diamine (0.430 g, 3.00 mmol), anhydrous 1,4-dioxane (20.0 mL), 3-[bis(propan-2-yl)carbarnoyl]propanoic acid (0,804 g 4.00 mmol), and polyphosphoric acid (2.35 g, 24.0 mmol), The reaction mixture was heated to 105° C. under argon with stirring for 20 hours. The reaction was quenched with water (30.0 mL) and sodium carbonate (12.3 g, 117 mmol) and partitioned into ethyl acetate. The organic layer was then concentrated and purified by Reverse-phase Medium Pressure Liquid Chromatography (50 g C18 column, methanol in 25.0 mmolar ammonium formate). The salt was partitioned between ethyl acetate and saturated sodium bicarbonate, washed with brine, dried over sodium sulfate, filtered, and concentrated to yield the product. This material was additionally purified by silica gel chromatography (0-5% methanol in dichloromethane) and recrystallized twice from dichloromethane and hexanes to yield the final product as a powder (0.157 g, 17%). ¹H NMR (500 MHz, DMSO-d₆) δ 13.12-12.51 (m, 1H), 8.22 (s, 1H), 8.00 (d, J=23.9 Hz, 1H), 4.04 (hept, J=6.3 Hz, 1H), 3.45 (s, 1H), 3.01 (t, J=7.4 Hz, 2H), 2.84 (dd, J=8.5, 6.4 Hz, 2H), 1.24 (d, J=6.7 Hz, 6H), 1.13 (d, J=6.6 Hz, 6H). APCI [M+H]calc'd=309.1476 observed=309.1494.

### Example 31

### 2-[(5,6-dichloro-1H-1,3-benzodiazol-2-yl)amino]-N,N-bis(propan-2-yl)acetamide

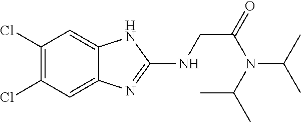 [SMILES: CC(C)N(C(=O)CNc1nc2cc(Cl)c(Cl)cc2[nH]1)C(C)C]

2-amino-N,N-bis(propari-2-yl)acetamide. 2-chloro-N,N-bis(propan-2-yl)acetamide (1.77 g, 10.0 mmol) was prepared according to the method described in Example 2, dissolved in dimethylformamide (20.0 mL), combined with sodium azide (1.95 g, 30.0 mmol), and heated to 50° C. under argon for 19 hours. The reaction mixture was cooled to room temperature, partitioned between ethyl acetate and water, washed with brine, 1.00 N aqueous hydrochloric acid, brine, dried over sodium sulfate, filtered and concentrated to yield the product as an oil (1.82 yield, 99%). ¹H NMR (500 MHz, Chloroform-d) δ 3.87 (s, 2H), 3.72 (h, J=6.7 Hz, 1H), 3.53 (s, 1H), 1.41 (d, J=6.8 Hz, 6H), 1.28-1.18 (m, 6H). Spectra match that reported for this compound in Eur. J. Med, Chem., 2013, 69, 338.

This material was dissolved in ethanol (30.0 mL), stirred with 10% palladium on carbon (0.304 g, 2.85 mmol). The reaction vessel was sealed and hydrogenated using a balloon filled with hydrogen for 14 hours. The reaction was filtered through Celite (washing with ethanol) and concentrated in vacuo to yield the product as an oil (1.50 g, 95%). ¹H NMR (500 MHz, Chloroform-d) δ 3.79 (dt, J=13.4, 6.7 Hz, 1H), 3.42 (d, J=18.0 Hz, 1H), 3.36 (s, 2H), 1.36 (d, J=6.8 Hz, 6H), 1.15 (d, J=6.6 Hz, 6H). Spectra match that reported in Monaghan, Sandra Marina; Mantell, Simon John, WO2000023457.

A 100 mL 24/40 round bottom flask was charged with 2-amino-N,N-bis(propan-2-yl)acetamide (0.158 g, 1.00 mmol), chlorobenzene (5.00 mL) and 2,5,6-trichloro-1H-benzo[d]imidazole (0.110 g, 0.500 mmol). Under an argon atmosphere, the flask was heated to 140° C. for 17 hours, during which time precipitate formed. The reaction was concentrated and purified by Reverse-phase Medium Pressure Liquid Chromatography (50 g C18 column, methanol in 25.0 mmolar ammonium formate). The resulting salt was partitioned between ethyl acetate and saturated sodium bicarbonate, and the organic layer washed with brine, dried over sodium sulfate, filtered, and concentrated in vacuo to yield the product. The product was further purified by recrystallization from dichloromethane and hexanes to yield the pure product as a white solid (0.122 g, 60%). ¹H NMR (500 MHz, DMSO-d₆) δ 10.86 (s, 1H), 7.32 (d, J=36.8 Hz, 2H), 6.85 (s, 1H), 4.11 (s, 2H), 3.91 (s, 1H), 3.50 (s, 1H), 1.30 (d, J=6.7 Hz, 6H), 1.16 (d, J=6.6 Hz, 6H). HRMS ESI (+) calc'd for [M+Na]=365.0911, found=365.0917.

### Example 32

### N,N-bis(propan-2-yl-3-(4,5,6,7-tetrafluoro-1H-1,3-benzodiazol-2-yl)propenamide

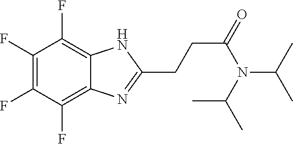 [SMILES: CC(C)N(C(=O)CCc1nc2c(F)c(F)c(F)c(F)c2[nH]1)C(C)C]

Tetrafluorobenzene-1,2-diamine. A roundbottom flask was charged with 2-nitrotetrafluoroaniline (0.420 g, 2.00 mmol), palladium on carbon (10%, 0.347 g) and ethanol (20.0 mL). The flask was evacuated and flushed with hydrogen (1 atmosphere) and allowed to stir for 20 hours. The reaction was then filtered through Celite and concentrated to yield tetrafluorobenzene-1,2-diamine (0.310 g, 86%), which was used without further purification. ¹H NMR (500 MHz, DMSO-d6): 4.92 (br s, 4H). ¹⁹F NMR (470 MHz, DMSO-d6) δ −163.92-164.11 (m), −177.84-178.00 (m).

N,N-bis(propan-2-yl)-3-(4,5,6,7-tetrafluoro-1H-1,3-benzodiazol-2-yl)propenamide. A 100 24/40 round bottom flask was charged with tetrafluorobenzene-1,2-diamine (0.310 g, 1.70 mmol), 3-[bis(propan-2-yl)carbamoyl]propanoic acid (0.608 g, 3.02 mmol), and 1,4-dioxane (12.0 mL). The reaction mixture was stirred until the solids dissolved and polyphosphoric acid added (3.08 g, 31.4 mmol). The reaction mixture was heated under argon for 22 hours, quenched with water (30.0 mL) and sodium carbonate (12.3 g, 117 mmol) and partitioned into ethyl acetate. The organic layer was then concentrated in vacuo and purified by Reverse-phase Medium Pressure Liquid Chromatography (50 g C18 column, methanol in 25.0 mmolar ammonium formate). The resulting salt was partitioned between ethyl acetate and a saturated sodium bicarbonate solution. The organic layer washed with brine, dried over sodium sulfate, filtered, and concentrated in vacuo. The product was additionally purified by recrystallization from dichloromethane/hexanes to yield the pure product as a tan powder (0.324 g, 55%). ¹H NMR (500 MHz, DMSO-d₆) δ 13.41 (s, 1H), 4.04 (p, J=6.8 Hz, 1H), 3.45 (s, 1H), 3.00 (t, J=7.4 Hz, 2H), 2.83 (t, J=7.4 Hz, 2H), 1.24 (d, J=6.7 Hz, 6H), 1.14 (d, J=6.6 Hz, 6H). ¹⁹F NMR (470 MHz, DMSO-d₆) δ −156.49 (d, J=408.5 Hz), −163.34 (d, J=886.4 Hz). HRMS ESI (+) calc'd for [M+Na]=368.1361, found=368,1344.

### Example 33

### 4-(5,6-dichloro-1H-1,3-benzodiazol-2-yl)-N,N-bis(propan-2-yl)butanamide

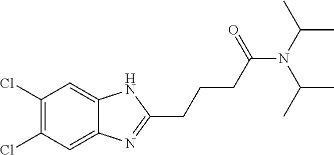 [SMILES: CC(C)N(C(=O)CCCc1nc2cc(Cl)c(Cl)cc2[nH]1)C(C)C]

4-(5,6-dichloro-1H-1,3-benzodiazol-2-yl)-N,N-bis(propan-2-yl)butanamide. 4-[bis(propan-2-yl)carbamoyl]butanoic acid was prepared by combining glutaric anhydride (1.16 g, 10.0 mmol) and diisopropyl amine (1.22 g, 11.0 mmol) and heating to 50° C. in ethyl acetate (30.0 mL) for 17 hours. The reaction mixture was cooled to room temperature and concentrated in vacuo to yield the crude 4-[bis(propan-2-yl)carbamoyl]butanoic acid (1.68 g, 78% yield). A 100 mL 24/40 round bottom flask was charged with 4,5-dichlorobenzene-1,2-diamine (0.354 g, 2.00 mmol), 4-[bis(propan-2-yl)carbamoyl]butanoic acid (0.645 g, 3.00 mmol), and 1,4-dioxane (12.0 mL). The reaction mixture was stirred until the solids dissolved and polyphosphoric acid was added (1.56 g, 16.0 mmol). The reaction mixture was heated to 105° C. under argon for 20 hours, quenched with water (30 mL) and sodium carbonate (12.3 g, 117 mmol) and partitioned with ethyl acetate. The organic layer was then concentrated and purified by Reverse-phase Medium Pressure Liquid Chromatography (50 g C18 column, methanol in 25.0 mmolar ammonium formate). The product was partitioned between ethyl acetate and saturated sodium bicarbonate, washed with brine, dried over sodium sulfate, filtered and concentrated. The product was additionally purified by recrystallization from dichloromethane/hexanes to yield the pure product as a solid (0.097 g, 14%). ¹H NMR (500 MHz, DMSO-d₆) δ 12.53 (s, 1H), 7,77 (s, 1H), 7.66 (s, 1H), 3,94 (p, J=6.7 Hz, 1H), 3.46 (br s, 1H), 2.83 (t, J=7.5 Hz, 2H), 2.33 (t, J=7.4 Hz, 2H), 1.95 (p, J=7.5 Hz, 2H), 1.26 (d, J=6.7 Hz, 6H), 1.08 (d, J=6.6 Hz, 6H). HRMS ESI (+) calc'd for [M+H]=356.1292, found=356.1303.

### Example 34

### 3-(5,6-dichloro-1H-1,3-benzodiazol-2-yl)-1-[(2R,6S)-2,6-dimethylpiperidin-1-yl]propan-1-one

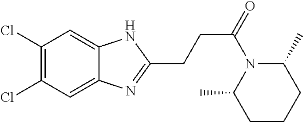 [SMILES: C[C@@H]1CCC[C@H](C)N1C(=O)CCc1nc2cc(Cl)c(Cl)cc2[nH]1]

3-(5,6-dichloro-1H-1,3-benzodiazol-2-yl)-1-[(2R6S)-2,6-dimethylpiperidin-1-yl]propan-1-one. A roundbottom flask was charged with cis-(2R,6S)-dimethylpiperidine (0.588 g, 5.20 mmol) and succinic anhydride (0.500 g, 5.00 mmol) in ethyl acetate (20.0 mL) then heated to 80° C. for 19 hours. The reaction mixture was cooled to room temperature and concentrated in vacuo to yield 4-[(2R,6S)-2,6-dimethylpiperidin-1-yl]-4-oxobutanoic acid a crude oil (1.15 g, 100%). The crude material was carried on to the next step without purification. A 100 mL 24/40 round bottom flask was charged with 4,5-dichlorobenzene-1,2-diamine (0.354 g, 2.00 mmol), crude 4-[(2R,6S)-2,6-dimethylpiperidin-1-yl]-4-oxobutanoic acid (0.645 g, 3.00 mmol), and 1,4-dioxane (12.0 mL). The reaction mixture was stirred until the solids dissolved and polyphosphoric acid was added (1.56 g, 16.0 mmol). The reaction mixture was heated to 105° C. under argon for 54 hours, quenched with water (30 mL) and sodium carbonate (117 mmol, 12.3 g), and partitioned into ethyl acetate. The organic layer was then concentrated and when redissolved in methanol a solid precipitated. This was filtered and the methanol-soluble material was purified by Reverse-phase Medium Pressure Liquid Chromatography (50 g C18 column, methanol in 25.0 mmolar ammonium formate). The product was partitioned between ethyl acetate and a saturated sodium bicarbonate solution and the organic layer washed with brine, dried over sodium sulfate, filtered, and concentrated in vacuo. The product was additionally purified by recrystallization from dichloromethane/hexanes to yield the pure product as a powder (0.395 g, 37%). ¹H NMR (500 MHz, DMSO-d₆) δ 12.49 (s, 1H), 7.72 (d, J=12.0 Hz, 2H), 4.54 (s, 1H), 4.15 (s, 1H), 3.11-2.86 (m, 3H), 3.37 (1H, s), 2.76 (dd, J=15.9, 8.1 Hz, 1H), 1.74 (qd, J=12.9, 11.4, 5.4 Hz, 1H), 1.61-1.34 (m, 5H), 1.29-1.14 (m, 3H), 1.06 (d, J=7.1 Hz, 3H). HRMS ESI (+) calc'd for [M+H]=354.1136, found=354.1140.

### Example 35

### 3-(7-chloro-1H-1,3-benzodiazol-2-yl)-N,N-bis(propan-2-yl)propenamide

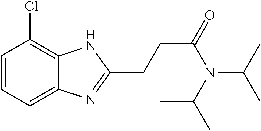 [SMILES: CC(C)N(C(=O)CCc1nc2cccc(Cl)c2[nH]1)C(C)C]

A 100 mL 24/40 round bottom flask was charged with 3-(0.427 g, 3.00 mmol), 3-[bis(propan-2-yl)carbamoyl]propanoic acid (0.804 g, 4.00 mmol), and 1,4-dioxane (10.0 mL). The reaction mixture was stirred until the solids dissolved and polyphosphoric acid added (1.17 g, 12.0 mmol). The reaction mixture was heated to 105° C. under an argon atmosphere for 17 hours, quenched with water (30.0 mL) and sodium carbonate (117 mmol, 12.3 g), then partitioned into ethyl acetate. The organic layer was then concentrated and redissolved in methanol. After 5 minutes a solid precipitated. This material was collected by filtration and the supernatant concentrated in vacuo and redissolved in ethyl acetate. A second crop of precipitate formed and was collected. The second crop was determined to be analytically pure (0.294 g, 32%). ¹H NMR (500 MHz, DMSO-d₆) δ 12.52 (d, J=48.0 Hz, 1H), 7.54-7.31 (m, 1H), 7.23-7.01 (m, 2H), 4.04 (ddd, J=18.3, 12.4, 6.9 Hz, 1H), 3.46 (br s, 1H), 3.01 (dd, J=8.7, 6.4 Hz, 2H), 2.82 (dd, J=8.6, 6.4 Hz, 2H), 1.26 (d, J=6.7 Hz, 6H), 1.14 (d, J=6.6 Hz, 6H). HRMS ESI (+) calc'd for [M+H]=308.1525, found=308.1546.

### Example 36

### 3-(5,6-dibromo-1H-1,3-benzodiazol-2-yl)-N,N-bis(propan-2-yl)propenamide

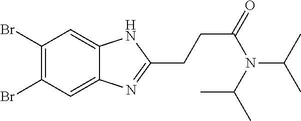 [SMILES: CC(C)N(C(=O)CCc1nc2cc(Br)c(Br)cc2[nH]1)C(C)C]

A 100 mL 24/40 round bottom flask was charged with 4,5-dibromobenzene-1,2-diamine (0.398 g, 1.50 mmol), 3-[bis(propan-2-yl)carbamoyl]propanoic acid (0.402 g, 2.00 mmol), and 1,4-dioxane (10.0 mL). The reaction mixture was stirred until the solids dissolved and polyphosphoric acid was added (1.17 g, 12.0 mmol). The mixture was heated to 105° C. under an argon atmosphere for 19 hours, quenched with water (30.0 mL) and sodium carbonate (117 mmol, 12.3 g), and then partitioned into ethyl acetate. The organic layer was then concentrated in vacuo and purified by Reverse-phase Medium Pressure Liquid Chromatography (50 g C18 column, methanol in 25.0 mmolar ammonium formate). The formate salt was then partitioned between ethyl acetate and saturated a sodium bicarbonate solution and the organic layer washed with brine, dried over sodium sulfate, filtered, and concentrated in vacuo. The product was additionally purified by recrystallization from dichlorormethane/hexanes to yield the pure product as a powder (0.296 g, 46%). ¹H NMR (500 MHz, DMSO-d₆) δ 12.47 (s, 1H), 7.85 (s, 2H), 4.03 (p, J=6.7 Hz, 1H), 3.45 (5, 1H), 2.99 (dd, J=8.5, 6.4 Hz, 2H), 2.81 (dd, J=8.5, 6.5 Hz, 2H), 1.25 (d, J=6.7 Hz, 6H), 1.13 (d, J=6.6 Hz, 6H). HRMS ESI (+) calc'd for [M+H]=430.0125, found=4300125.

### Example 37

### 3-(6-bromo-5-chloro-1H-1,3-benzodiazol-2-yl-N,N-bis(propan-2 yl)propenamide

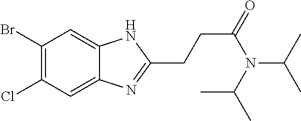 [SMILES: CC(C)N(C(=O)CCc1nc2cc(Cl)c(Br)cc2[nH]1)C(C)C]

4-Bromo-5-chlorobenzene-1,2-diamine. This material was prepared by the method described in Lars BARFACKER, Raimund Kast, Nils Griebenow, Heinrich Meier, Peter Kolkhof, Barbara ALBRECHT-KIPPER, Adam Nitsche, Johannes-Peter Stasch, Dirk Schneider, Nicole Teusch, Joachim Rudolph, James Whelan, William Bullock, Susan Pleasic-Williams, WO2010020363. 4-Bromo-5-chloro-2-nitroaniiine (0.950 g, 3.77 mmol, was dissolved in ethanol (24.0 mL), and tin(II) chloride dihydrate (3.38 g, 15.1 mmol) subsequently added and the mixture heated to reflux for 20 hours. The reaction mixture was cooled to room temperature, concentrated in vacuo, and partitioned between ethyl acetate and a saturated aqueous sodium bicarbonate solution. A white precipitate formed in the aqueous layer, which was separated from the organic layer. The organic layer was washed with brine, dried over sodium sulfate, and concentrated in vacuo. The crude material was purified by silica gel chromatography (1:9 methanol:dichloromethane) to yield the product as a solid (0.780 g, 95%). ¹H NMR (500 MHz, DMSO-d6) δ 6.73 (s, 1H), 6.62 (s, 1H), 4.86 (s, 4H).

3-(6-bromo-5-chloro-1H-1,3-benzodiazol-2-yl)-N,N-bis(propan-2yl)propenamide. A 100 mL 24/40 round bottom flask was charged with 4-bromo-chlorobenzene-1,2-diamine (0.300 g, 1.30 mmol), 3-[bis(propan-2-yl)carbamoyl]propanoic acid (0.402 g, 2.00 mmol), and 1,4-dioxane (8.00 mL). The reaction mixture was stirred until the solids dissolved and treated with polyphosphoric acid (2.0 g, 20.0 mmol). The reaction mixture was heated to 105° C. under an argon atmosphere for 19 hours, quenched with water (30 mL) and sodium carbonate (117 mmol, 12.3 g), then partitioned into ethyl acetate. The organic layer was then concentrated in vacuo and purified by Reverse-phase Medium Pressure Liquid Chromatography (50 g C18 column, methanol in 25.0 mmolar ammonium formate). The formate salt was partitioned between ethyl acetate and a saturated sodium bicarbonate solution, and the organic layer washed with brine, dried over sodium sulfate, filtered and concentrated in vacuo. The product was additionally purified by recrystallization from dichloromethane/hexanes to yield the pure product as a powder (0.255 g, 51%). ¹H NMR (500 MHz, DMSO-d₆) δ 12.,48 (s, 1H), 7.78 (dd, J=57.7, 32.5 Hz, 2H), 4.03 (dd, J13.5, 7.0 Hz, 1H), 3.46 (br s, 1H), 2.98 (t, 7.3 Hz, 2H), 2.80 (t, J=7.4 Hz, 2H), 1.24 (d, J=6.7 Hz, 6H), 1.12 (d, J=6.6 Hz, 6H). HRMS ESI (+) calc'd for [M+H]=386.0631, found=386.0627.

### Example 38

### 3-(5,6-dichloro-1H-1,3-benzodiazol-2-yl-1-(4-methylpiperazin-1-yl)propan-1-one

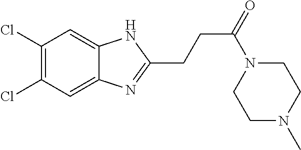 [SMILES: CN1CCN(C(=O)CCc2nc3cc(Cl)c(Cl)cc3[nH]2)CC1]

3-(5,6-dichloro-1H-1,3-benzodiazol-2-yl)-1-(4-methylpiperazin-1-yl)propan-1-one. 4-(4-Methyipiperazin-1-yl)-4-oxobutanoic acid was prepared (Banik, B., K. Becker, F., F., Bioorg. Med. Chem., 2001, 9, 593) by combining N-methylpiperazine (1.05 g, 10.0 mmol) and succinic anhydride (1.00 g, 10.0 mmol) in ethyl acetate (25.0 mL) and heating to 70° C. for 19 hours. The reaction mixture was cooled to room temperature and concentrated in vacuo to yield the product as a crude oil (2.17 g, 100%), and was carried on to the next step without further purification. A 100 mL 24/40 round bottom flask was charged with 4,5-dichlorobenzene-1,2-diamine (0.531 g, 3.00 mmol), crude 4-(4-methylpiperazin-1-yl)-4-oxobutanoic acid (0.802 g, 4.00 mmol), and 1,4-dioxane (12.0 mL). The reaction mixture was stirred until the solids dissolved and then subsequently treated with polyphosphoric acid (1.56 g, 16.0 mmol). The reaction mixture was heated to 105° C., under an argon atmosphere for 20 hours, quenched with water (30 mL) and sodium carbonate (12.3 g, 117 mmol), and then partitioned into ethyl acetate. The organic layer was then concentrated in vacuo and then redissolved in methanol, a solid precipitate forming in 5 minutes. The solid was filtered and the methanol-soluble material purified by Reverse-phase Medium Pressure Liquid Chromatography (50 g C18 column, methanol in 25.0 mmolar ammonium formate). The salt was partitioned between ethyl acetate and saturated sodium bicarbonate, and the organic layer washed with brine, dried over sodium sulfate, filtered, and concentrated in vacuo. The product was additionally purified by recrystallization from dichloromethane/hexanes to yield the pure product as a powder (0.133 g, 13%). ¹H NMR (500 MHz, DMSO-d₆) δ 12.49 (s, 1H), 7.76 (s, 1H), 7.68 (s, 1H), 3.44 (dd, J=12.7, 7.7 Hz, 4H), 3.02 (t, J=7.2 Hz, 2H), 2.86 (t, J=7.2 Hz, 2H), 2.27 (t, J=5.0 Hz, 2H), 2.20 (t, J=5.2 Hz, 2H), 2.15 (s, 3H). HRMS ESI (+) calc'd for [M+H]=341,0932, found=341.0928.

### Example 39

### 3-(5,6-dichloro-1H-1,3-benzodiazol-2-yl)-1-(piperidin -1-yl)propan-1-one

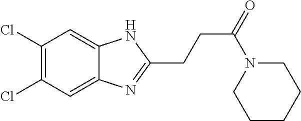 [SMILES: O=C(CCc1nc2cc(Cl)c(Cl)cc2[nH]1)N1CCCCC1]

4-Oxo-4-(piperidin-1-yl)butandic acid. A round bottom flask was charged with piperidine (0.340 g, 4.00 mmol) and succinic anhydride (0.400 g, 4.00 mmol), then dissolved in ethyl acetate (15.0 mL) and heated to 70° C. for 19 hours. The reaction mixture was cooled to room temperature and concentrated in vacuo to yield the product as an oil (0.740 g, 100%, Banik, B., K.; Becker, F., F., Bioorg. Med. Chem., 2001, 9, 593.). ¹H NMR (500 MHz, DMSO-d₆) δ 12.00 (s, 1H), 3.43-3.34 (m, 4H), 2.49 (d, J=13.4 Hz, 2H), 2.43-2.38 (m, 2H), 1.60-1.53 (m, 2H), 1.48 (tg, J=8.7, 5.2, 4.3 Hz, 2H), 1.43-1.36 (m, 2H).

3-(5,6-dichloro-1H-1,3-benzodiazol-2-yl)-1-(piperidin-1-yl)propan-one. A 100 mL 24/40 round bottom flask was charged with 4,5-dichlorobenzene-1,2-diamine (0.442 g, 2.50 mmol), 4-oxo-4-(piperidin-1-yl)butanoic acid (0.648 g, 3.50 mmol), and 1,4-dioxane (10.0 mL). The reaction mixture was stirred until the solids dissolved and subsequently treated polyphosphoric acid (2.60 g, 26.5 mmol). The reaction mixture was heated to 105° C. under an argon atmosphere for 22 hours, quenched with water (30 mL) and sodium carbonate (117 mmol, 12.3 g), and then partitioned into ethyl acetate. The organic layer was then concentrated and purified by Reverse-phase Medium Pressure Liquid Chromatography (50 g C18 column, methanol in 25.0 mmolar ammonium formate). The product was then partitioned between ethyl acetate and a saturated sodium bicarbonate solution. The organic layer washed with brine, dried over sodium sulfate, filtered, and concentrated in vacuo. The product was additionally purified by recrystallization from dichloromethane/hexanes to yield the pure product as a powder (0.150 g, 18%). ¹H NMR (500 MHz, DMSO-d₆) δ 12.58 (d, J=90.6 Hz, 1H), 7.84-7.63 (m, 2H), 3.41 (q, J=5.5 Hz, 4H), 3.02 (t, J=7.3 Hz, 2H), 2.86 (q, J=8.2, 7.3 Hz, 2H), 1.56 (q, J=5.8 Hz, 2H), 152-1.44 (m, 2H), 1.38 (dd, J=10.9, 5.7 Hz, 2H). HRMS ESI (4) calc'd for [M+H]=326.0823, found=326.0822.

### Example 40

### 1-(azepan-1-yl)-3-(5,6-dichloro-1H-1,3-benndiazol-2-yl)propan-1-one

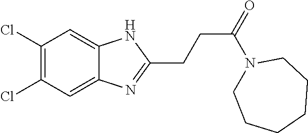 [SMILES: O=C(CCc1nc2cc(Cl)c(Cl)cc2[nH]1)N1CCCCCC1]

4-(Azepan-1-yl)-4-oxobutanoic add. A round bottom flask was charged with hexamethyleneimine (0.505 g, 5.10 mmol), succinic anhydride (0.500 g, 5.00 mmol) and ethyl acetate (20.0 then heated to 70° C. for 26 hours. The reaction mixture was then cooled to room temperature and concentrated in vacuo to yield the product as an oil (1.31 g, 100%, Elina M. Jarho, Erik A. A. Wallén, Johannes A. M. Christiaans, Markus M. Forsberg, Jarkko I. Venäläinen, Pekka T. Männistö, Jukka Gynther, and Antti Poso, J. Med. Chem., 2005, 48, 4772.). ¹H NMR (500 MHz, DMSO-d₆) δ 11.82 (s, 1H), 3.44-3.39 (m, 2H), 3.38-3.34 (m, 2H), 2.51-2.45 (m, 2H), 2.42-2.36 (m, 2H), 1.69-1.62 (m, 2H), 1.58-1.51 (m, 2H), 1.51-1.39 (m, 4H).

1-(azepan-1-yl)-3-(5,6-dichloro-1H-1,3-benzodiazol-2-yl)propan-1-one. A 100 mL 24/40 round bottom flask was charged with 4,5-dichlorobenzene-1,2-diamine (0.531 g, 3.00 mmol), 4-(azepan-1-yl)-4-oxobutanoic acid (0.797 g, 4.00 mmol), and 1,4-dioxane (10.0 mL). The reaction mixture was stirred until the solids dissolved and then treated with polyphosphoric acid (2.87 g, 30.0 mmol). The mixture was heated to 105° C. under an argon atmosphere for 52 hours, quenched with water (30 mL) and sodium carbonate (117 mmol, 12.3 g), and then partitioned into ethyl acetate. The organic layer was then concentrated, dissolved in methanol, and purified by Reverse-phase Medium Pressure Liquid Chromatography (50 g C18 column, methanol in 25.0 mmolar ammonium formate). The product was partitioned between ethyl acetate and a saturated sodium bicarbonate solution. The organic layer washed with brine, dried over sodium sulfate, filtered, and concentrated in yawn. The product triturated during concentration of the organic layer to yield the pure product as a solid (0.474 g, 46%). ¹H NMR (500 MHz, DMSO-d₆) δ 12.49 (s, 1H), 7.71 (d, J=32.6 Hz, 2H), 3.45 (t, J=6.1 Hz, 2H), 3.41-3.37 (m, 2H), 3.03 (t, J=7.2 Hz, 2H), 2.85 (t, J=7.3 Hz, 2H), 1.67 (p, J=5.9 Hz, 2H), 1.58-1.52 (m, 2H), 1.52-1.40 (m, 4H). ESI (+) calc'd for [M+H]=340.0979, found=340.0977.

### Example 41

### 3-(5,6-dichloro-1H-1,3-benzodiazol-2-yl)-N,N-bis(2-methylpropyl) propenamide

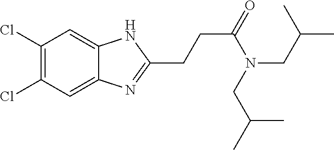 [SMILES: CC(C)CN(CC(C)C)C(=O)CCc1nc2cc(Cl)c(Cl)cc2[nH]1]

3-[bis(2-methylpropyl)carbamoyl]propandic acid. A round bottom flask was charged with diisobutylamine (0.659 g, 5.10 mmol), succinic anhydride (0.500 g, 5.00 mmol) and ethyl acetate (20.0 mL). The mixture heated to reflux under an argon atmosphere for 18 hours. The material was then concentrated in vacuo to yield an oil (1.34 g, 100%, Oh, H.; Han, S. K.; Kim, B. H., Heteroatom Chemistry, 2012, 23, 187.). ¹H NMR (500 MHz, DMSO-d6) δ 11.90 (s, 1H), 3.09 (dd, J=10.5, 7.5 Hz, 4H), 2.50 (dd, J=7.3, 2.5 Hz, 2H), 2.41 (dd, J=7.1, 5.1 Hz, 2H), 1.90 (heptd, J=7.0, 3.5 Hz, 2H), 0.86 (d, J=6.6 Hz, 6H), 0.78 (d, J=6.8 Hz, 6H).

3-(5,6-dichloro-1H-1,3-benzodiazol-2-yl)-N,N-bis(2-methylpropyl)propenamde. A 100 mL 24/40 round bottom flask was charged with 4,5-dichlorobenzene-1,2-diamine (0.442 g, 2.50 mmol), 3-[bis(2-methylpropyl)carbamoyl]propanoic acid (0.802 g, 3.50 mmol), and 1,4-dioxane (10.0 mL). The mixture was stirred until the solids dissolved, then treated with polyphosphoric acid (2.55 g, 26.0 mmol). The mixture was then heated to 105° C. under an argon atmosphere for 52 hours, then quenched with water (30 mL) and sodium carbonate (12.3 g, 117 mmol), and partitioned into ethyl acetate. The organic layer was then concentrated and purified by Reverse-phase Medium Pressure Liquid Chromatography (50 g C18 column, methanol in 25.0 mmoiar ammonium formate). The formate salt was then partitioned between ethyl acetate and saturated sodium bicarbonate. The organic layer washed with brine, dried over sodium sulfate, filtered, and concentrated in vacuo. The product was further purified by recrystallization from dichoromethane/hexanes to yield the product as a tan powder (0.465 g, 50%). ¹H NMR (500 MHz, DMSO-d₆) δ 12.48 (s, 1H), 7.71 (d, J=20.2 Hz, 2H), 3.13 (d, J=7.6 Hz, 2H), 3.08 (d, J=7.5 Hz, 2H), 3.04 (t, J=7.2 Hz, 2H), 2.85 (t, J=7.2 Hz, 2H), 1.89 (tp, J=13.7, 6.8 Hz, 2H), 0.86 (d, J=6.6 Hz, 6H), 0.74 (d, J=6.6 Hz, 6H). HRMS ESI (+) calc'd for [M+H]=370.1449, found=370.1471.

### Example 42

### 5,6-dichloro-N-(cyclohexylmethyl)-1H-1,3-benzodiazol-2-amine

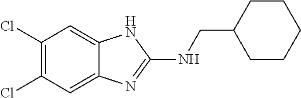 [SMILES: Clc1cc2nc(NCC3CCCCC3)[nH]c2cc1Cl]

A 100 mL 24/40 round bottom flask was charged with 2,5,6-trichloro-1H-1,3-benzodiazole (0.221 g, 1.00 mmol), chlorobenzene (5.00 mL), and cyclohexanemethylamine (0.339 g, 3.00 mmol). The mixture was then heated to 140° C. under an argon atmosphere for 24 hours, after which it was cooled to room temperature and concentrated in vacuo. The crude material was then purified by Reverse-phase Medium Pressure Liquid Chromatography (50 g C18 column, methanol in 25.0 mmoiar ammonium formate). The salt was then partitioned between ethyl acetate and a saturated sodium bicarbonate solution. The organic layer was then washed with brine, dried over sodium sulfate, filtered, and concentrated in vacuo. The recovered material was additionally purified by silica gel chromatography (0 to 5% methanol in dichloromethane). to yield the product (0.170 g, 57%). ¹H NMR (500 MHz, DMSO-d₆) δ 10.87 (s, 1H), 7.24 (s, 1 H), 7.00 (s, 1H), 3.09 (t, J=6.4 Hz, 2H), 1.75-1.63 (m, 4H), 1.63-1.48 (m, 2H), 1.23-1.06 (m, 4H), 0.96-0.84 (m, 2H). HRMS ESI (+) calc'd for [M+H]=298.0874, 298.0874.

### Example 43

### 2-[(5,6-dichloro-1H-1,3-benzodiazol-2-yl)oxy]-N,N-bis(propan-2-yl)acetamide

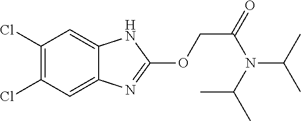 [SMILES: CC(C)N(C(=O)COc1nc2cc(Cl)c(Cl)cc2[nH]1)C(C)C]

2-hydroxy-N,N-bis(propan-2-yl)acetamide. 2-chloro-N,N-bis(propan-2-yl)acetamide (2.27 g, 12.7 mmol) was dissolved in tetrahydrofuran (40.0 mL) and benzyl alcohol (1.62 g, 15.0 mmol). The reaction mixture was cooled to 0° C. under argon and sodium hydride was added (0.537 g, 16.0 mmol). The reaction mixture was heated to 50° C. for 19 hours, at which time it was quenched with a solution of saturated aqueous ammonium chloride (20.0 mL) and partitioned with ethyl acetate. The aqueous layer was extracted with ethyl acetate, the organic layers were combined, washed with brine, dried over sodium sulfate, filtered, and concentrated in vacuo to yield the crude product as an oil. The product was purified by silica gel chromatography (1:4 ethyl acetate in hexanes) to yield the pure product as an oil (2.89 g, 91%, Miyatake, Tsuneo; Tanaka, Shigeyuki; Shimada, Atsuo, JP49011412 B.). ¹H NMR (500 MHz, Chloroform-d) δ7.41-7.24 (m, 5H), 4.60 (s, 2H), 4.10 (s, 2H), 4.03-3.92 (m, 1H), 3.48-3.35 (m, 1H), 1.42 (d, J=6.8 Hz, 6H), 1.17 (dd, J=11.0, 6.4 Hz, 6H). This material was dissolved in ethanol (30.0 mL) and stirred with 10% palladium on carbon (0.442 0, 4.15 mmol) under a hydrogen atmosphere applied with a balloon for 20 hours. The reaction mixture was filtered through celite (washing with ethanol) and concentrated in vacuo to yield the product as a white solid (1.59 g, 91%, Scardovi, N.; Casalini, A.; Peri, F.; Righi, P., Org. Lett., 2002, 4, 965.). 1H NMR (500 MHz, Chloroform-d) δ4.07 (d, J=4.2 Hz, 2H), 3.60 (dt, J=13.3, 6.6 Hz, 2H), 3.48 (p, J=6,7 Hz, 1H), 1.41 (d, J=6.7 Hz, 6H), 1.19 (d, J=6.5 Hz, 6H).

2,5,6-trichloro-1-{([2-(trimethylsilyl)ethoxy]methyl}-1H-1,3-benzodiazole. This material was prepared according to the general method described in Duane Burnett, Wen-Lian Wu, Thavalakulamgara Sasikumar, William Greenlee, Mary Caplen, Tao Guo, Rachael Hunter, US20050054628. A 100 mL 24/40 roundbottom flask was charged with 2,5,6-trichloro-1H-1,3-benzodiazole (0.424 g, 1.90 mmol) and dimethylformamide (10.0 mL). Under an argon atmosphere, sodium hydride was added (0.100, g, 3.0 mmol), and once gas evolution was complete, 2-(chloromethoxy)ethyl trimethylsilane (0.416 g, 2.50 mmol) was added via syringe. The mixture was stirred for 19 hours at which time water was added (20.0 mL). The mixture was extracted with ethyl acetate, the organic layer washed with brine, dried over sodium sulfate, decanted and concentrated in vacuo to yield the crude product which was purified by silica gel chromatography (3:7 ethyl acetate/hexanes) to yield the product (0.510 g, 76%). ¹H NMR (500 MHz, DMSO-d ₆) δ8.12 (s, 1H), 7.95 (s, 1H), 5.64 (s, 2H), 3.54 (t, J=7.9 Hz, 2H), 0.81 (t, J=7.9 Hz, 2H), −0.12 (s, 9H).

2-[(5,6-dichloro-1-{[2-(trimethylsilyl)ethoxy]methyl}-1H-1,3-benzodiazol-2-yl)oxy]-N,N-bis(propan-2-yl)acetamide. A 50 mL 24/40 round bottom flask was charged with 2-hydroxy-N,N-bis(propan-2-yl)acetamide (0.318 g, 2.00 mmol) and tetrahydrofuran (5.00 ml). The mixture was cooled to 0° C., under an argon atmosphere, and sodium hydride added (0.100 g, 3,00 mmol). Once gas evolution stopped, 2,5,6-trichloro-1-{[2-(trimethylsilyl)ethoxy]methyl}-1H-1,3-benzodiazole (0.476 g, 1.30 mmol) was added as a solution in tetrahydrofuran (5.00 mL). The reaction was warmed to room temperature and stirred for 19 hours, at which time an aqueous solution of saturated ammonium chloride (20.0 mL) was added. The reaction was extracted with ethyl acetate and the organic layer washed with brine, dried over sodium sulfate, decanted, and concentrated in vasuo to yield the crude product. This material was purified by silica gel chromatography (1:1 ethyl acetate/hexanes) to yield the final product as a white solid (0.510 g, 83%). 1H NMR (500 MHz, DMSO-d6) δ7.75 (s, 1H), 7.64 (s, 1H), 5.44 (s, 2H), 5.22 (s, 2H), 3.83 (p, J=6.6 Hz, 1H), 3.64 3.56 (m, 2H), 3.48 (tt, J=13.4, 7.3 Hz, 1H), 1.24 (d, J=6.7 Hz, 6H), 1.17 (d, J=6.4 Hz, 6H), 0.88-0.78 (m, 2H), −0.09 (s, 9H).

2-[(5,6-dichloro-1H-1,3-benzodiazol-2-yl)oxy]-N,N-bis(propan-2-yl)acetamide. 2-[(5,6-dichloro-1-{[2-(trimethylsilyl)ethoxy]methyl}-1H-1,3-benzodiazol-2-yl)oxyl-N,N-bis(propan-2-yl)acetamide was dissolved in tetrahydrofuran (10.0 mL) under an argon atmosphere and tetrabutylammonium fluoride was added as a 1.00 M solution in tetrahydrofuran (10.0 mL), followed by 0.050 mL of water. The reaction mixture was heated to 50° C. for 70 hours at which time it was concentrated in vacuo. The crude material was purified by Reverse-phase Medium Pressure Liquid Chromatography (50 g C18 column, methanol in 25.0 mmolar ammonium formate). The product was partitioned between ethyl acetate and saturated sodium bicarbonate. The organic layer was then washed with brine, dried over sodium sulfate, filtered, and concentrated in vacuo. The product was additionally purified by silica gel chromatography (15:85 methanol/dichloromethane) to yield an oil. This material was recrystallized from dichloromethane/hexanes to yield the product as white needles (0.092 g, 26%). ¹H NMR (500 MHz, DMSO-d₆) δ 12.37 (s, 1H), 7.71 7.38 (m, 2H), 5.14 (s, 2H), 3.83 (p, J=6.7 Hz, 1H), 3.50 (dt, J=13.9, 4.8 Hz, 1H), 1.23 (dd, J=39.5, 6.6 Hz, 12H). HRMS ESI (+) calc'd for [M+H]=344.0928, 344.0941.

### Example 44

### 1-[(1H-1,3-benzodiazo-2-yl)methyl]-3,3-bis(propan-2-yl)urea

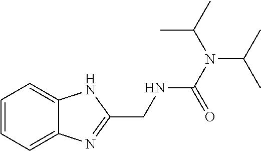 [SMILES: CC(C)N(C(=O)NCc1nc2ccccc2[nH]1)C(C)C]

A 100 mL 24/40 round bottom flask was charged with 1H-benzo[d]imidazoyl-methanamine (0.294 g, 2.00 mmol), tetrahydrofuran (5.00 mL), and triethylamine (0.253 g, 2.50 mmol) under an argon atmosphere. Diisopropylcarbarnoyl chloride (0.359 g, 2.20 mmol), dissolved in tetrahydrofuran (5.00 mL), was added to the reaction mixture in a dropwise fashion. The mixture was stirred for 2 hours at room temperature, filtered, and washed with ethyl acetate. The filtrate was concentrated in vacuo and purified by Reverse-phase Medium Pressure Liquid Chromatography (50 g C18 column, methanol in 25.0 mmolar ammonium formate). The product was partitioned between ethyl acetate and saturated sodium bicarbonate, and the organic layer washed with brine, dried over sodium sulfate, filtered, and concentrated in vacuo. The product was additionally purified by silica gel chromatography (0 to 5% methanol in dichloromethane) to yield a solid. This material was recrystallized from dichloromethane/hexanes to yield the product as a powder (0.171 g, 31%). ¹H NMR (500 MHz, DMSO-d6) δ11.96 (s, 1H), 7.56-7.41 (m, 2H), 7.11 (dt, J=6.0, 3.6 Hz, 2H), 6.59 (t, J=5.4 Hz, 1H), 4.39 (d, J=5.4 Hz, 2H), 3.74 (p, J=6.9 Hz, 2H), 1.18 (d, J=6.7 Hz, 12H). HEMS ESI (+) calc'd for [M+H]=275.1868, found=275.1881.

### Example 45

### 2-cyclohexyl-1-(5,6-dichloro-1H-1,3-benzodiazol-2-yl)ethan-1-one

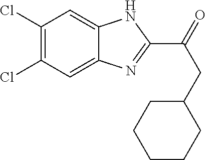 [SMILES: O=C(CC1CCCCC1)c1nc2cc(Cl)c(Cl)cc2[nH]1]

5,6-Dichloro-1-{[2-(trimethylsilyl)ethoxy]methyl}-1H-1,3-benzodiazole. A 100 mL 24/40 round bottom flask was charged with 5,6-dichlorobenzimidazole (1.17 g, 6.30 mmol) and dimethylformamide (15.0 mL), then cooled to 0° C. and treated with sodium hydride (0.302 g, 9.0 mmol). The mixture was stirred under an argon atmosphere and 2-(trimethylsilyi)ethoxymethyl chloride (1.33 g, 8.00 mmol) added. The mixture was warmed to room temperature and stirred for 19 hours, then quenched with a saturated aqueous ammonium chloride solution (20.0 mL). The reaction mixture was extracted with ethyl acetate, the organic layers combined, washed with brine, dried over sodium sulfate, filtered, and concentrated in vacuo. The crude material was purified by silica gel chromatography (0 to 100% ethyl acetate in hexanes). yield a solid (1.54 g, 76%). ¹H NMR (500 MHz, DMSO-d6) δ 8.49 (s, 1H), 8.01 (s, 1H), 7.97 (s, 1H), 5.63 (s, 2H), 3.52-3.42 (m, 2H), 0.86-0.77 (m, 2H), −0.11 (s, 9H).

2-Cyclohexyl-1-(5,6-dichloro-1-{[2-(trimethylsilyl)ethoxy]methyl}-1H-1,3-benzodiazol-2-yl)ethan-1-one. A 250 mL 24/40 roundbottom flask was flushed with argon, charged with tetrahydrofuran (10.0 mL), diisopropylamine (0.303 g, 3.00 mmol), cooled to −78° C., and n-butyllithium added (1.0 ml 2.50 mmol). The reaction was stirred for 10 minutes at −78° C. and 5,6-dichloro-1-{[2-(trimethylsilyl)ethoxy]methyl}-1H-1,3-benzodiazole (0.634 g, 2.00 mmol) was added as a solution in tetrahydrofuran (5.00 mL). The reaction mixture was stirred for 30 minutes at −78° C. and ethyl cyclohexyl acetate (0.681 g, 4.00 mmol) added as a solution in tetrahydrofuran (5.00 mL). After 2 hours at 78° C. the reaction mixture was warmed to room temperature and quenched with saturated aqueous ammonium chloride (30.0 mL). The biphasic mixture was stirred at room temperature for 30 minutes, partitioned, and the aqueous layer was extracted with ethyl acetate. The organic layers were combined, washed with brine, dried over sodium sulfate, filtered, and concentrated in vacuo. The crude material was purified by silica gel chromatography (0 to 20% ethyl acetate in hexanes) to yield a clear oil (0.180 g, 20%). ¹H NMR (500 MHz, DMSO-d6) δ8.22 (s, 1H), 8.18 (s, 1H), 5.97 (s, 2H), 3.49-3.44 (m, 2H), 3,08 (d, J=6.9 Hz, 2H), 1.90 (th, J=10.5, 3.4 Hz, 1H), 1.72-1.53 (m, 5H), 1.27-1.07 (m, 3H), 1.07-0.96 (m, 2H), 0.80-0.73 (m, 2H), −0.15 (s, 9H),

2-Cyclonexyl-1-(5,6-dichloro-1H-1,3-benzodiazol-2-yl)ethan-1-one. A 25 mL 14/20 round bottom flask was charged with 2-cyclohexyl-1-(5,6-dichloro-1-{[2-(trimethylsilyl)ethoxy]methyl}-1H-1,3-benzodiazol-2-yl)ethan-1-one (0.180 g, 0.200 mmol), ethanol (10.0 mL), and concentrated aqueous hydrochloric acid (1.00 mL, 12.0 mmol). The reaction mixture was heated to 80° C. for 2 hours during which time a solid precipitated. The mixture was cooled to room temperature, the solid was filtered and washed with hexanes. The solid was suspended in ethyl acetate and stirred with a saturated aqueous sodium bicarbonate solution until the organic layer cleared. The biphasic mixture was partitioned and the organic layer washed with brine, dried over sodium sulfate, filtered, and concentrated in vacuo to yield the pure product as a solid (0.086 g, 69%). ¹H NMR (500 MHz, DMSO-d₆) δ 13.58 (s, 1H), 7.94 (d, J=190.5 Hz, 2H), 3.03 (d, J=6.9 Hz, 2H), 1.94 (ddt, J=18.2, 10.8, 3.2 Hz, 1H), 1.63 (dtd, J=24.9, 14.4, 7.4 Hz, 5H), 1.28-1.07 (m, 3H), 1.01 (qd, J=13.8, 13.1, 3.8 Hz, 2H), HRMS ESI (+) calc'd for [M+H]=311.0714, found=311.0717.

### Example 46

### 1-[(5,6-dichloro-1H-1,3-benzodiazol-2-yl)methyl]-3,3-bis(propan-2-yl)urea

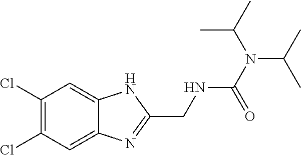 [SMILES: CC(C)N(C(=O)NCc1nc2cc(Cl)c(Cl)cc2[nH]1)C(C)C]

A 100 mL 24/40 roundbottom flask was charged with 1-(5,6-dichloro-1H-1,3-benzodiazoi-2-yl)methanamine (0.238 g, 1.10 mmoi), tetrahydrofuran (5.00 mL), and triethylamine (0.202 g, 2.00 mmol). The reaction mixture was stirred and flushed with argon. In a separate vial diisopropylcarbamoyl chloride (0.229 g, 1.40 mmol) was dissolved in tetrahydrofuran (5.00 mL) and added to the solution by syringe. The mixture was stirred at 40° C. for 22 hours, concentrated in vacuo, and purified by Reverse-phase Medium Pressure Liquid Chromatography (50 g C18 column, methanol in 25.0 mmolar ammonium formate). The product was partitioned between ethyl acetate and a saturated sodium bicarbonate solution. The organic layer washed with brine, dried over sodium sulfate, filtered, and concentrated in vacuo. The product was additionally purified by silica gel chromatography (0 to 5% methanol in dichloromethane) and triturated from dichloromethane/methanol and hexanes, filtered, and dried in vacuo to yield a solid (0.253 g, 67%). ¹H NMR (500 MHz, DMSO-d₆) δ 12.23 (s, 1H), 7.78 (s, 1H), 7.71 (s, 1H), 6.66 (t, J=5.4 Hz, 1H), 4.38 (d, J=5.5 Hz, 2H), 3.73 (p, J=5.7 Hz, 2H), 1.18 (d, J=6.7 Hz, 12H), HRMS ESI (±) calc'd for [M+H]=343.1088, found=343.1109.

### Example 47

### (2E)-3-(5,6-dichloro-1H-1,3-benzodiazoi-2-yl)-N,N-bis(propan-2-yl)prop-2-enamide

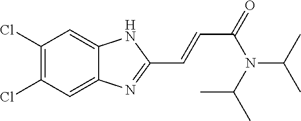 [SMILES: CC(C)N(C(=O)/C=C/c1nc2cc(Cl)c(Cl)cc2[nH]1)C(C)C]

Ethyl (2E)-3-[bis(propan-2-yl)carbamoyl]prop-2-enoate. A 100 mL 24/40 roundbottorn flask was charged with ethyl hydrogen furnarate (2.88 g, 20.0 mmol), dichloromethane (80.0 mL), and after cooling to 0° C. with stirring thionyl chloride (11.8 g, 100 mmol). The reaction was warmed to room temperature and stirred for 70 hours, at which time it was concentrated in vacuo to yield a yellow oil. Dichloromethane was added (40.0 mL) and the reaction mixture was concentrated in vacuo again. This process was repeated two more times to yield a solid which was dissolved in dichloromethane (100 mL), cooled to 0° C., and diisopropylamine (3.61 g, 35.7 mmol) was added dissolved in dichloromethane (5.00 mL). The reaction was stirred for 15 minutes at 0° C., transferred to a reparatory funnel, washed with aqueous 1.00 N hydrochloric acid (50.0 mL), saturated aqueous sodium bicarbonate (50.0 mL), dried over sodium sulfate, filtered and concentrated in vacuo to yield the product (2.17 g, 96%). ¹H NMR (500 MHz, Chloroform-d) δ7.35 (d, J=15.4 Hz, 1H), 6.61 (d, J=15.5 Hz, 1H), 4.23 (q, J=7.1 Hz, 2H), 3.98 (hept, J=6.7 Hz, 1H), 3.77-3.63 (m, 1H), 1.38 (d, J=6.8 Hz, 6H), 1.30 (d, J=7.1 Hz, 3H), 1.28-1.16 (m, 6H).

(2E)-3-[bis(propan-2-yl)carbamoyl]prop-2-enoic acid. A 100 mL 24/40 roundbottom flask was charged with ethyl (2E)-3-[bis(propan-2-yl)carbamoyl]prop-2-enoate (2.10 g, 9.20 mmol), ethanol (40.0 mL), water (10.0 mL), and solid sodium hydroxide (0.799 g, 20,0 mmol). The reaction mixture was stirred and heated to 60° C. for 20 hours. The reaction mixture was concentrated n vacuo and partitioned between ethyl acetate (50.0 mL) and 1.00 N aqueous hydrochloric acid (50.0 mL). The organic layer was washed with brine, dried over sodium sulfate, filtered, and concentrated in vacuo to yield the product (0.833 g, 49%). ¹H NMR (500 MHz, DMSO-d6) δ 12.81 (s, 1H), 7.26 (d, J=15.5 Hz, 1H), 6.31 (d, J=15.5 Hz, 1H), 3.96 (hept, J=7.1 Hz, 1H), 3.71-3.58 (m, 1H), 1.28 (d, J=6.8 Hz, 6H), 1.15 (d, J=6.7 Hz, 6H).

(2E)-3-(5,6-dichioro-1H-1,3-benzodiazol-2-yl)-N,N-bis(propan-2-yl)prop-2-enamide. A 100 mL 24/40 roundbottom flask was charged with 4,5-dichlorobenzene-1,2-diamine (0.354 g, 2.00 mmol), (2E)-3-[bis(propan-2-yl)carbarnoyl]prop-2-enoic add (0.597 g, 3.00 mmol), dioxane (8.00 mL), and polyphosphoric acid (1.22 g, 12.4 mmol). The reaction mixture was stirred and heated to 110° C. under argon for 20 hours, cooled to room temperature, quenched with water and extracted with ethyl acetate. The organic layer was washed with brine, dried over sodium sulfate, filtered, and concentrated in vacuo. The resulting residue was purified by Reverse-phase Medium Pressure Liquid Chromatography (50 g C18 column, methanol in 25.0 mmolar ammonium formate). The product was then partitioned between ethyl acetate and a saturated sodium bicarbonate solution. The organic layer was washed with brine, dried over sodium sulfate, filtered, and concentrated in vacuo. The product was additionally purified by silica gel chromatography (1:1 ethyl acetate:hexanes), and finally purified by recrystallization from dichloromethane/hexanes to yield the product as a solid (0.150 g, 22%). ¹H NMR (500 MHz, DMSO-d6) δ 13.10 (s, 1H), 7.91 (s, 1H), 7.81 (s, 1H), 7.51 (d, J=15.5 Hz, 1H), 7,27 (d, J=15.5 Hz, 1H), 4.17 (5, 1H), 3.74 (s, 1H), 1.31 (d, J=6.6 Hz, 6H), 1.23 (d, J=6.8 Hz, 6H). HRMS ESI (−) calc'd for [M+H]=340.0979, found=340.1025.

### Example 48

### 1-[(5,6-dichloro-1H-1,3-benzodiazol-2-yl)sulfanyl]-N,N-bis(2-methylpropyl)formamide

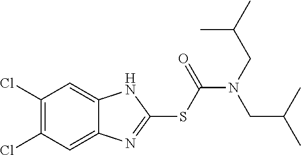 [SMILES: CC(C)CN(CC(C)C)C(=O)Sc1nc2cc(Cl)c(Cl)cc2[nH]1]

1-[(5,6-dichloro-1H-1,3-benzodiazol-2-yl)sulfanyl]-N,N-bis(2-methylpropyl)formamide. A 25.0 mL 14/20 roundbottom flask was charged with 5,6-dichloro-1H-1,3-benzodiazole-2-thiol (0.245 g, 1.10 mmol), flushed with argon, and dimethylformamide was added (3.00 mL). Triethylamine was added (0.202 g, 2.0 mmol) followed by N,N-bis(2-methylpropyl)carbamoyl chloride (0.287 g, 1.50 mmol, prepared and used crude by the method of Biediger, R. J.; Gundlach, C. W.; Market, R. V.; Savage, M. M.; Vanderslice, P.; WO2012068251) as a solution dissolved in dimethylformamide (0.500 mL). The reaction was stirred at room temperature for 18 hours, at which time it was partitioned between ethyl acetate and brine. The organic layer was washed with water and brine, dried over sodium sulfate, filtered, and concentrated in vacuo to yield the crude product as a solid. The crude material was purified by Reverse-phase Medium Pressure Liquid Chromatography (50 g C18 column, methanol in 25.0 mmolar ammonium formate). The product was partitioned between ethyl acetate and a saturated sodium bicarbonate solution. The organic layer washed with brine, dried over sodium sulfate, filtered, and concentrated in vacuo. The product was additionally purified by silica gel chromatography (0 to 5% methanol in dichloromethane) and triturated from dichloromethane/methanol and hexanes, filtered, and dried in vacuo to yield the product as a solid (0.209 g, 51%). ¹H NMR (500 MHz, DMSO-d6) δ 13.22 (s, 1H), 7.97-7.72 (m, 2H), 3.18 (d, J=7.5 Hz, 4H), 2.14-1.94 (m, 2H), 0.94 (d, J=6.6 Hz, 6H), 0.83 (d, J=6.5 Hz, 6H). HRMS ESI (+) [M+H] calc'd=374.0855, found=374.0856.

### Example 49

### 1-(5,6-dichloro-1H-1,3-benzodiazol-2-yl)-3,3-bis(2-methylpropyl)urea

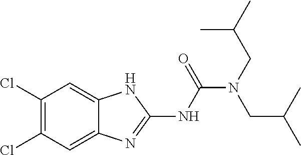 [SMILES: CC(C)CN(CC(C)C)C(=O)Nc1nc2cc(Cl)c(Cl)cc2[nH]1]

1-(5,6-dichloro-1H-1,3-benzodiazol-2-yl)-3,3-bis(2-methylpropyl)urea. A 25.0 mL 14/20 roundbottom flask was charged with 5,6-dichloro-1H-1,3-benzodiazol-2-amine (0.140 g, 0.690 mmol), dimethylformamide (3.00 mL), cooled to 0° C. and sodium hydride (0.033 g, 1,00 mmol) was added. The vessel was flushed with argon and after 5 minutes N,N-bis(2-methylpropyl)carbarnoyl chloride (0.191 g, 1.00 mmol, Biediger, R. J.; Gundlach, C. W.; Market, R. V.; Savage, M. M.; Vanderslice, P.; WO2012068251) was added as a solution dissolved in dimethylformamide (0.500 mL). The reaction mixture was heated to 45° C. for 19 hours, cooled to room temperature, partitioned between ethyl acetate and brine, washed with water and brine, dried over sodium sulfate, filtered, and concentrated in vacuo. A solid precipitated which was filtered and washed with hexanes. The supernatant was concentrated in vacuo and purified by silica gel chromatography (0 to 10% methanol in dichloromethane). The pure product was obtained by crystallization from methanol (0.014 g, 6%). ¹H NMR (500 MHz, DMSO-d6) δ 7.40 (s, 1H), 7.06 (s, 1H), 6.93 (s, 2H), 3.38 (br s, 2H), 2.96 (dd, J=13.7, 7.8 Hz, 2H), 1.97 (s, 2H), 0.82 (s, 12H). and HRMS ESI (+) [M+H]calc'd=357.1243, found=357,1265.

### Example 50

### N-(5,6-dichloro-1H-1,3-benzodiazol-2-yl)azepane-1-carboxamide

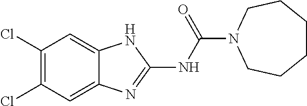 [SMILES: O=C(Nc1nc2cc(Cl)c(Cl)cc2[nH]1)N1CCCCCC1]

A 25.0 mL 14/20 roundbottom flask was charged with 5,6-dichloro-1H-1,3-benzadiazol-2-amine (0.250 g, 1.20 mmol), dimethylformamide (5.00 mL), cooled to 0° C. and sodium hydride (0.050 g, 1.50 mmol) was added. The vessel was flushed with argon and after 5 minutes azepane-1-carbonyl chloride (0.304 g, 1.80 mmol, prepared and used crude according to the method described in Biediger, R. J.; Gundlach, C. W.; Market, R. V.; Savage, M. M.; Vanderslice, P.; WO2012068251) was added neat. The reaction mixture was heated to 45° C. for 19 hours, cooled to room temperature, partitioned between ethyl acetate and brine, washed with water and brine, dried over sodium sulfate, filtered, and concentrated in vacuo. The crude product was purified by Reverse-phase Medium Pressure Liquid Chromatography (50 g C18 column, methanol in 25.0 mmolar ammonium formate). The product was then partitioned between ethyl acetate and saturated sodium bicarbonate solution, washed with brine, dried over sodium sulfate, filtered and concentrated in vacuo. The pure product was obtained as a tan powder by recrystallization from dichloromethanethexanes (0.167 g, 43%). ¹H NMR (500 MHz, Chloroform-d) δ7.46 (s, 1H), 7.12 (s, 1H), 5.65-5.47 (m, 2H). 3,75 (s, 2H), 3.32 (ddd, J=13.3, 8.4, 4.3 Hz, 2H), 2.00-1.48 (m, 12H). and HRMS ESI (+) [M+H] calc'd=327.0774, found=327.0790.

### Example 51

### Cyclohexyl N-(5,6-dichloro-1H-1,3-benzodiazol-2-yl)carbamate

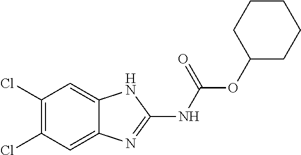 [SMILES: O=C(Nc1nc2cc(Cl)c(Cl)cc2[nH]1)OC1CCCCC1]

A 25.0 mL 14/20 roundbottom flask was charged with 5,6-dichloro-1H-1,3-benzodiazol-2-amine (0.200 g, 1.00 mmol), dimethylformamide (3.00 mL), cooled to 0° C. and sodium hydride (0.042 g, 1.25 mmol) was added. The vessel was flushed with argon and after 5 minutes cyclohexyl carbonochloridate (0.243 g, 1.50 mmol, prepared according and used crude using the method described in Biediger, R. J.; Gundlach, C. W.; Market, R. V.; Savage, M. M.; Vanderslice, P.; WO2012068251) was added neat. The reaction mixture was heated to 45° C. for 20 hours, cooled to room temperature, partitioned between ethyl acetate and brine, washed with water and brine, dried over sodium sulfate, filtered, and concentrated vacuo. The pure product was obtained as powder by recrystallization from dichloromethane/hexanes (0.175 g, 53%). NMR (500 MHz, DMSO-d6) δ8.00-7.10 (m, 4H), 5.01 (s, 1H), 2.14-1.10 (m, 10H). HRMS ESI (+) calc'd=328.0614, found=328.0623.

### Example 52

### 1-(5,6-dichloro-1H-1,3-benzbdiazol-2-yl)-3,3-bis(propan-2-yl)urea

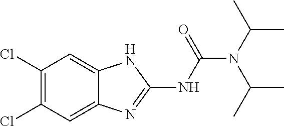 [SMILES: CC(C)N(C(=O)Nc1nc2cc(Cl)c(Cl)cc2[nH]1)C(C)C]

A 10 mL 14/20 roundbottorn flask was charged with a stir bar, 5,6-dichloro-1H-1,3-benzodiazol-2-amine (0.202 g, 1.00 mmol), and dimethylformamide (3.00 mL). The reaction mixture was stirred and sodium hydride was added (0.040 g, 1.20 mmol). After gas evolution had ceased, the reaction vessel was sealed and flushed with argon. Diisopropyl carbamoyl chloride (0.212 g, 1.30 mmol) was added as a solution in dimethylformamide (2.00 mL). The reaction mixture was heated to 45° C. for 16 hours, diluted with ethyl acetate, washed with brine, water, and brine. The organic layer was dried over sodium sulfate, filtered, and concentrated in vacuo. The crude product was purified by Reverse-phase Medium Pressure Liquid Chromatography (50 g C18 column, methanol in 25.0 mmolar ammonium formate). The product was then partitioned between ethyl acetate and saturated sodium bicarbonate solution, washed with brine, dried over sodium sulfate, filtered and concentrated in vacuo. The product was additionally purified by silica gel chromatography (0 to 10% methanol in dichloromethane). The pure product was obtained as a powder by recrystallization from dichloromethane/hexanes (0.104 g, 32%). ¹H NMR (500 MHz, DMSO-d6) δ 7.37 (s, 1H), 7.10 (s, 1H), 6.87 (s, 2H), 3.61 (s, 2H), 1.29 (5, 12H). HRMS ESI (+) calc'd for [M+H]=329.0932, found=329.0947.

### Example 53

### 3-(6-chloro-5-(trifluoromethoxy)-1H-benzo[d]imidazol-2-yl)-N,N-diisopropylpropanamide

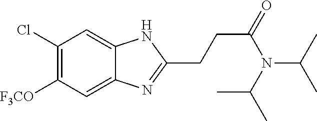 [SMILES: CC(C)N(C(=O)CCc1nc2cc(OC(F)(F)F)c(Cl)cc2[nH]1)C(C)C]

A 100 mL 24/40 roundbottom flask was charged with a stir bar, 4-Chloro-5-(trifluoromethoxy)-1,2-benzenediamine (0.226 g, 1.00 mmol), 3-[bis(propan-2-yl)carbamoyi]propanoic acid (0.241 g, 1.20 mmol), 1,4-dioxane (4.00 mL) and polyphosphoric acid (0.970 g, 9.89 mmol). The reaction vessel was sealed, flushed with argon and heated to 105° C. for 22 hours. The reaction mixture was cooled to room temperature and quenched with the addition of water (10.0 mL) and sodium carbonate (10.5 g, 100 mmol). The reaction mixture was washed with ethyl acetate (3×). The organic layers were combined, washed with brine, dried over sodium sulfate, filtered and concentrated in vacuo to yield the crude product. This material was purified by Reverse-phase Medium Pressure Liquid Chromatography (50 g C18 column, methanol in 25.0 mmolar aqueous ammonium formate). The product was then partitioned between ethyl acetate and saturated sodium bicarbonate solution, washed with brine, dried over sodium sulfate, filtered and concentrated in vacuo. The product was additionally purified by silica gel chromatography (0 to 10% methanol in dichloromethane). The pure product was 10 obtained as a tan powder by recrystallization from dichloromethane/hexanes (0.085 g, 22%). 1H NMR (500 MHz, DMSO-d6) δ 12.58 (s, 1H), 7.83-7.55 (m, 2H), 4.03 (p, J=6.4 Hz, 1H), 147 (d, J=17.4 Hz, 1H), 3.00 (t, J=7.4 Hz, 2H), 2.81 (dd, J=3.4, 6.5 Hz, 2H), 1.24 (d, J=6.7 Hz, 6H), 1.12 (d, J=6.6 Hz, 6H). 19F NMR (470 MHz, DMSO-d6) δ −57.34 (d, J=24.5 Hz). HAMS ESI (+) Calc'd for [M+Na]=414.1172, found=414.1182.

### Example 54

### 3-(6-chloro-5-(trifluoromethyl)-1H-benzo[d]imidazol-2-yl)-N,N-diisopropylpropanamide

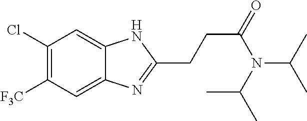 [SMILES: CC(C)N(C(=O)CCc1nc2cc(C(F)(F)F)c(Cl)cc2[nH]1)C(C)C]

A 100 mL 24/40 roundbottom flask was charged with a stir bar, 4-Chloro-5-(trifluoromethyl)-1,2-benzenediamine (0.211 g, 1.00 mmol), 3-[bis(propan-2yl)carbamoyl]propanoic acid (0.298 g, 1.20 mmol), 1,4-dioxane (5.00 mL) and polyphosphoric acid (0.970 g, 9.89 mmol). The reaction vessel was sealed, flushed with argon and heated to 105° C. for 21 hours. The reaction mixture was cooled to room temperature and quenched with the addition of water (10.0 mL) and sodium carbonate (10.5 g, 100 mmol). The reaction mixture was washed with ethyl acetate (3×). The organic layers were combined, washed with brine, dried over sodium sulfate, filtered and concentrated in vacuo to yield the crude product. This material was purified by Reverse-phase Medium Pressure Liquid Chromatography (50 g C18 column, methanol in 25.0 mmolar aqueous ammonium formate). The product was then partitioned between ethyl acetate and saturated sodium bicarbonate solution, washed with brine, dried over sodium sulfate, filtered and concentrated in vacuo. The product was additionally purified by silica gel chromatography (0 to 10% methanol in dichloromethane). The pure product was obtained as a tan powder by recrystallization from dichloromethane/hexanes (0.102 g, 27% yield) 1H NMR (500 MHz, DMSO-d6) δ 12.75 (d, J=12.4 Hz, 1H), 8.00-7.69 (m, 2H), 4.03 (p, J=6.7 Hz, 1H), 3,45 (s, 1H), 3,03 (q, J=7.2 Hz, 2H), 2.83 (t, J=7.4 Hz, 2H), 1.24 (d, J=6,7 Hz, 6H), 1.13 (d, J=6.6 Hz, 6H). 19F NMR (470 MHz, DMSO-d6) δ −59.04, −59.20 (d, J=4.0 Hz), −59.62 (d, J=3.7 Hz). HRMS ESI (+) Calc'd for [M+Na]=398.1223, found=398.1245.

### Example 55

### Minimum Inhibitory Concentration (MIC) Studies

MIC determination against (1) Mtb H37Rv, (2) a rifampin resistant (RIF:rpoBS450L) strain (ATCC #35838), (3) an isoniazid resistant (INH: katGdel) strain (MS015), (4) a moxifloxacin resistant (MOX: gyrD94K) strain (MOX3), (5) M. avium (ATCC 700891 MAC 101) and (6) M. abscessus (ATCC 19977) in microdilution assays was carried out for the compounds from Examples 2, 21A, 19:

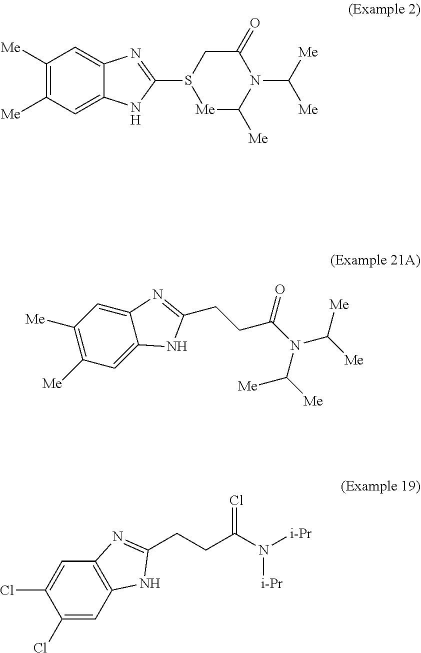

Isoniazid (INH) and moxifloxacin (MOX) were used as controls in the Mtb studies. Clarithromycin, rifampicin and amikacin were used as controls in the nontuberculous mycobacteria (NTM) studies. MIC testing against Mtb H37Rv M. abscessus demonstrated that all three compounds had activity in the susceptible range (≤4 mg/L) in the concentration ranges tested. Two compounds had activity in the intermediate range (4-32 mg/L) against M. avium. MIC values for all test compounds plus positive controls are listed in Table 1.

TABLE 1

| | | | | | | M. avium |
|---|---|---|---|---|---|---|
| | | H37Rv | H37Rv | H37Rv | M. abcessus | 700891 |
| Compound | H37Rv | (rpoB^{S450L}) | (katG^{del}) | (gyrA^{D94K}) | 19977 | (MAC 101) |
| Example 2 | 0.5 | 0.25 | 0.5 | 0.5 | 4 | >64 |
| Example 21A | 0.25 | 0.25 | ≤0.125 | ≤0.125 | 2 | 16 |
| Example 19 | ≤0.125 | ≤0.125 | ≤0.125 | ≤0.125 | 1 | 8 |

Assay performance (reliability and repeatability) was assessed by repeat studies of a select number of compounds and concordance between the methods used to determine the MIC. These analyses confirmed concordance with both MIC calls for this study and all repeats were correlative as well.

### Example 54

### IC₅₀ Studies

The compounds of Examples 2, 21A, and 19 were tested for 1) for ex vivo efficacy and 2) cytotoxicity. Ex vivo efficacy was determined by inhibition of Mycobacterium tuberculosis (Mtb) H37Rv, Mycobacterium avium complex (ATCC 700891 MAC 101), and Mycobacterium abscessus (ATCC 19977). Cytotoxicity tests were carried out against three human cell lines, THP-1 (ATCC TIB-202), HepG2 (ATCC HB-8065), and HeLa (ATCC CCL-2), IC₅₀ values were calculated from dose response curves to determine cytotoxicity of test compounds. This resulted in a total of 18 tests completed for the compounds of Examples 2, 21A, and 19. Of the three compounds tested for ex vivo efficacy against NTM strains, there was no activity (>32 mg/L) against M. avium and one compound with activity against M. abscessus. See Table 2. Cytotoxicity testing against THP-1 cells determined that all compounds had an IC₅₀ value >32 mg/L and compounds had an average therapeutic index (ratio of IC₅₀:MIC) of 29.9-339. These compounds were not cytotoxic (>128 mg/L) against HepG2 and HeLa cells. The compounds average therapeutic index for HepG2 was 12.4-48.3 and 96.7-389 for HeLa.

TABLE 2

| | M. avium ex | M. abscessus ex | | | |
|---|---|---|---|---|---|
| | vivo efficacy | vivo efficacy | THP-1 | HepG2 | HeLa |
| | (>90% inhibition | (>90% inhibition | Cytotoxicity | Cytotoxicity | Cytotoxicity |
| Compound | μg/ml) | μg/ml) | (IC50 μg/ml) | (IC50 μg/ml) | (IC50 μg/ml) |
| Example 2 | 0.5 | 0.25 | 0.5 | 0.5 | 4 |
| Example 21A | 0.25 | 0.25 | ≤0.125 | ≤0.125 | 2 |
| Example 19 | ≤0.125 | ≤0.125 | ≤0.125 | ≤0.125 | 1 |

### Example 56

### In Vivo Studies

Mice (C57Bl/6 mice) were aerosol infected with 200 CFU of Mtb Erdman and treated with compounds by oral gavage 5 day/week for two weeks. The mice were treated with the compounds of the formulae:

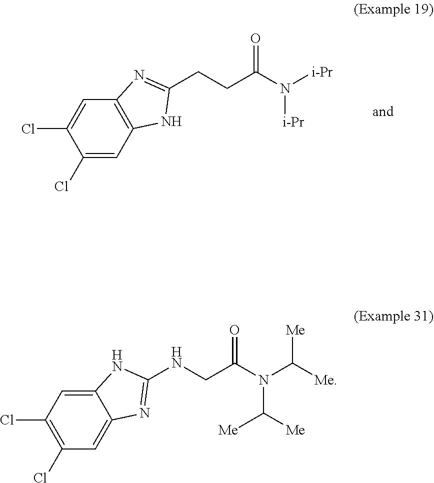 [SMILES: CC(C)N(C(=O)CCc1nc2cc(Cl)c(Cl)cc2[nH]1)C(C)C ; CC(C)N(C(=O)CNc1nc2cc(Cl)c(Cl)cc2[nH]1)C(C)C]

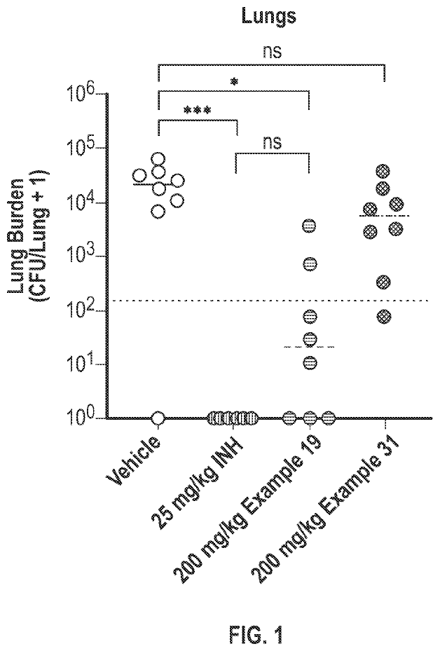

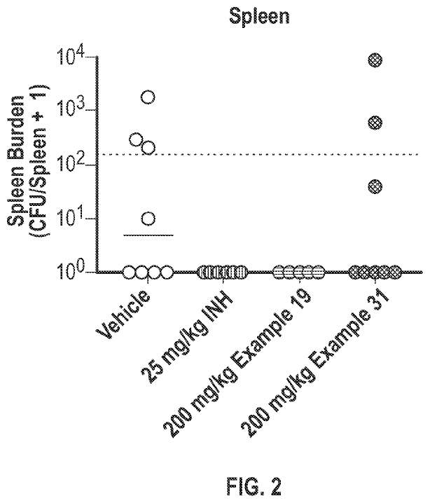

The lung and spleen burdens were measured for each compound and compared with vehicle, as well as with isoniazid (INH). The data are shown in FIGS. 1 and 2.

### Example 57

### N-(5,6-dichloro-1H-benzo[d]imidazol-2-yl)-4-phenyltetrahydro-2H-pyran-4-carboxamide

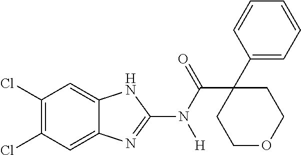 [SMILES: O=C(Nc1nc2cc(Cl)c(Cl)cc2[nH]1)C1(c2ccccc2)CCOCC1]

A 100 mL 24/40 roundbottom flask was charged with a stir bar, 4-phenyl tetrahydro-2H-pyran-4-carboxylic acid (0.123 g, 0.600 mmol), N,N-dimethylformamide (1.00 mL), triethylamine (0.101, 1.00 mmol), and 1-[Bis(dimethylamino)methylene]-1H-1,2,3-triazolo[4,5-b]pyridiniurn 3-oxide hexafluorophosphate (0.266 g, 0.700 g). The reaction vessel was sealed, flushed with argon and 5,6-dichloro-1H-benzo[d]imidazole-2-amine (0.160 g, 0.790 mmol) was added dissolved in N,N-dimethylformamide (1.00 mL). The reaction mixture was stirred for 20 hours, quenched with ethyl acetate, washed with brine, aqueous sodium bicarbonate, and brine. This material was purified by Reverse-phase Medium Pressure Liquid Chromatography (50 g C18 column, methanol in 25.0 mmolar aqueous ammonium formate). The product was then partitioned between ethyl acetate and saturated sodium bicarbonate solution, washed with brine, dried over sodium sulfate, filtered and concentrated in vacuo. The pure product was obtained as a tan powder by recrystallization from dichloromethane/hexanes (0.079 g, 34%). 1H NMR (500 MHz, DMSO-d6) δ 12.44 (s, 1H), 11.38 (s, 1H), 7.60 (s, 2H), 7.44 (d, J=7.7 Hz, 2H), 7.38 (t, J=7.7 Hz, 2H), 7.27 (t, J=7.2 Hz, 1H), 3.79 (dq, J=13.2, 5.2, 4.5 Hz, 2H), 3.54 (t, J=11.1 Hz, 2H), 2.66 (d, J=13.7 Hz, 2H), 1.99 (d, J=15.8 Hz, 2H). HRMS ESI (+) Calc'd for [M+H]=390.0772, found=390.0792.

### Example 58

### N-(5,6-dichloro-1H-benzo[d]imidazol-2-yl)-1-phenylcyclohexane-1-carboxamide

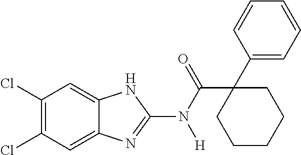 [SMILES: O=C(Nc1nc2cc(Cl)c(Cl)cc2[nH]1)C1(c2ccccc2)CCCCC1]

A 100 mL 24/40 roundbottom flask was charged with a stir bar, 1-phenyl-1-cyclohexyl carboxylic acid (0.265 g, 1.30 mmol), N,N-dimethylformamide (2.00 mL), triethylamine (0.303 g, 3.00 mmol), and 1-[Bis(dimethylamino)methylene]-1H-1,2,3-triazolo[4,5-b]pyridinium 3-oxide hexafluorophosphate (0.532 g, 1.40 mmol). The reaction vessel was sealed, flushed with argon and 5,6-dichloro-1H-benzo[d]imidazole-2-amine (0.306 g, 1.50 mmol) was added dissolved in N,N-dirnethylformamide (1.00 mL). The reaction mixture was stirred for 20 hours, quenched with ethyl acetate, washed with brine, aqueous sodium bicarbonate, and brine. This material was purified by Reverse-phase Medium Pressure Liquid Chromatography (50 g C18 column, methanol in 25.0 mmolar aqueous ammonium formate). The product was then partitioned between ethyl acetate and saturated sodium bicarbonate solution, washed with brine, dried over sodium sulfate, filtered and concentrated in vacuo. The product was filtered through a plug of silica (ethyl acetate) and the pure product was obtained as a tan powder by recrystallization from dichloromethane/hexanes (0.054 g, 10% yield). 1H NMR (500 MHz, Chloroform-d) δ 11.05 (s, 1H), 8.62 (s, 1H), 7.50 (d, J=33.6 Hz, 2H), 7.42-7.35 (m, 4H), 7.31 (td, J=5.7, 2.6 Hz, 1H), 2.36 (dd, J=13.5, 6.6 Hz, 2H), 2.14-2.03 (m, 2H), 1.62 (d, J=14.4 Hz, 5H), 1.47-1.39 (m, 1H), HRMS ESI (+) Calc'd for [M+H]=388,0979, found=388,0990.

### Example 59

### N-(5,6-dichlombenzo[d]thiazol-2-yl)-1-phenylcyclopentane-1-carboxamide

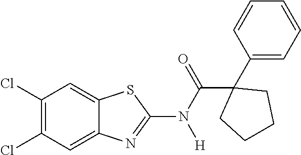 [SMILES: O=C(Nc1nc2cc(Cl)c(Cl)cc2s1)C1(c2ccccc2)CCCC1]

A 100 mL 24/40 roundbottom flask was charged with a stir bar, 1-phenyl-1-cyclopentyl carboxylic acid (0.190 g, 1.00 mmol), triethylamine (0.235 g, 2.50 mmol), N,N-dimethylformamide (2.00 mL), and 1-[Bis(dimethylamino)methylene]-1H-1,2,3-triazolo[4,5-b]pyridinium 3-oxide hexafluorophosphate (0.418 g, 1.10 mmol). The reaction vessel was sealed, flushed with argon, and 2-amino-5,6-dichlorobenzothiazole (0.262 g, 1.20 mmol) was added dissolved in N,N-dimethylformamide (1.00 mL). The reaction mixture was stirred for 20 hours, quenched with ethyl acetate, washed with brine, aqueous sodium bicarbonate, and brine. The organic layer was dried over sodium sulfate, filtered, and concentrated in vacuo. This material was purified by Reverse-phase Medium Pressure Liquid Chromatography (50 g C18 column, acetonitrile in 25.0 mmolar aqueous ammonium formate). The product was then partitioned between ethyl acetate and saturated sodium bicarbonate solution, washed with brine, dried over sodium sulfate, filtered and concentrated in vacuo to yield the pure produce (0.030 g, 8% yield). 1H NMR (500 MHz, DMSO-d6) δ 12.27 (s, 1H), 8.32 (s, 1H), 7.91 (s, 1H), 7.37 (dd, J=19.3, 7.6 Hz, 4H), 7.24 (t, J=7.1 Hz, 1H), 2.65 (d, J=7.2 Hz, 2H), 1.99 (d, J=14.2 Hz, 2H), 1.64 (d, J=33.4 Hz, 4H). HRMS ESI (+) Calc'd for [M+H]=391.0434, found=391.0438.

### Example 60

### N-(5,6-dichloro-1H-benzo[d]imidazol-2-yl)-1-methylcyclohexane-1-carboxamide

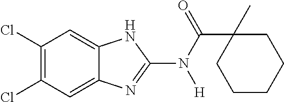 [SMILES: CC1(C(=O)Nc2nc3cc(Cl)c(Cl)cc3[nH]2)CCCCC1]

A 100 mL 24/40 roundbottom flask was charged with a stir bar, 1-methyl-1-cyclohexyl carboxylic acid (0.142 g, 1.00 mmol), triethylamine (0.303 g, 3.00 mmol), N,N-dimethylformamide (2.00 mL), and 1-[Bis(dimethylamino)methylene]-1H-1,2,3-triazolo[4,5-b]pyridinium 3-oxide hexafluorophosphate (0.418 g, 1.10 mmol). The reaction vessel was sealed, flushed with argon, and 5,6-dichloro-benzo[d]-imidazol-2-amine (0.242 g, 1.20 mmol) was added dissolved in N,N-dimethylformamide (1.00 mL). The reaction mixture was stirred for 20 hours, quenched with ethyl acetate, washed with brine, aqueous sodium bicarbonate, and brine. The organic layer was dried over sodium sulfate, filtered, and concentrated in vacuo. This material was purified by Reverse-phase Medium Pressure Liquid Chromatography (50 g C18 column, methanol in 25.0 mmolar aqueous ammonium formate), The product was then partitioned between ethyl acetate and saturated sodium bicarbonate solution, washed with brine, dried over sodium sulfate, filtered and concentrated in vacuo. This material was additionally recrystallized from dichloromethane/hexanes to obtain the pure product (0.069 g, 21% yield). 1H NMR (500 MHz, DMSO-d6) δ 12.39 (s, 1H), 11.29 (s, 1H), 7,62 (d, J=9.1 Hz, 2H), 2,12 (d, J=11.7 Hz, 2H), 1.56-1.25 (m, 8H), 1.21 (s, 3H). HRMS ESI (+) Calc'd for [M+H]=326.0823, found=326,0838.

### Example 61

### N-(5,6-dichlorobenzo[d]thiazol-2-yl)-1-phenylcyclohexane-1-carboxamide

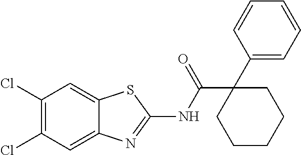 [SMILES: O=C(Nc1nc2cc(Cl)c(Cl)cc2s1)C1(c2ccccc2)CCCCC1]

A 100 mL 24/40 roundbottom flask was charged with a stir bar, 1-phenyl-1-cyclohexyl carboxylic acid (0.306 g, 1.50 mmol), triethylamine (0.405, 4.00 mmol), N,N-dimethylformamide (2.00 mL), and 1-[Bis(dimethylamino)methylene]-1H-1,2,3-triazolo[4,5-b]pyridinium 3-oxide hexafluorophosphate (0.646 g, 1.70 mmol). The reaction vessel was sealed, flushed with argon, and 2-amino-5,6-dichlorobenzothiazole (0.416 g, 1.90 mmol) was added dissolved in N,N-dimethylformamide (1.00 mL). The reaction mixture was stirred for 24 hours, quenched with ethyl acetate, washed with brine, aqueous sodium bicarbonate, and brine. The organic layer was dried over sodium sulfate, filtered, and concentrated in vacuo. This material was purified by Reverse-phase Medium Pressure Liquid Chromatography (50 g C18 column, acetonitrile in 25.0 mmolar aqueous ammonium formate). The product was then partitioned between ethyl acetate and saturated sodium bicarbonate solution, washed with brine, dried over sodium sulfate, filtered and concentrated in vacuo. This material was additionally purified by silica gel chromatography (0 to 10% methanol in dichioromethane) to yield the pure product (0.042 g, 7% yield). 1H NMR (500 MHz, Chloroform-d) δ 8.73 (s, 1H), 7.87 (s, 1H), 7.74 (s, 1H), 7.47-7.38 (m, 4H), 7.32 (ddt, J=7.3, 6.2, 1.8 Hz, 1H), 2.39 (dd, J=12.3, 6.5 Hz, 2H), 2.16-2.08 (m, 2H), 1.69-1.57 (m, 5H), 1.45 (dd, J=8.0, 4.5 Hz, 1H). HRMS ESI (+) Calc'd for [M+H]=405.0591, found=405.0595.

### Example 63

### N-(5,6-dichloro-1H-benzo[d]imidazol-2-yl)-1-(1H-pyrazol-1-yl)cyclohexane-1-carboxamide

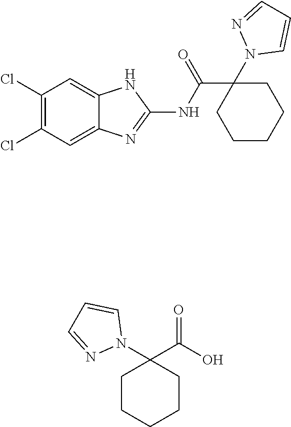 [SMILES: O=C(Nc1nc2cc(Cl)c(Cl)cc2[nH]1)C1(n2cccn2)CCCCC1 ; O=C(O)C1(n2cccn2)CCCCC1]

This compound was prepared according to the procedure published by Butcher, K. J., Hurst, J. Tett. Lett., 2009, 50, 2497. A 100 mL 24/40 roundbottom flask was charged with a stir bar, tetrahydrofuran (40.0 mL) and cooled to 0° C. The reaction vessel was seated and flushed with argon, pyrazole (0.408 g, 6.00 mmol) was added, followed by freshly powdered sodium hydroxide (1.16 g, 30.0 mmol) and cyclohexanone (1.76 g, 18.0 mmol). The reaction mixture was stirred and chloroform (3.58 g, 30.0 mmol). The reaction was stirred for 20 hours and warmed to room temperature. The solid was filtered, dissolved in water (50.0 mL) and extracted twice with diethyl ether. The aqueous layer was acidified to pH ˜3.00 with glacial acetic acid, extracted with ethyl acetate, dried over sodium sulfate, filtered, and concentrated to dryness in vacuo. The residue was recrystallized from dichloromethane/hexanes to provide the final product as a white solid (0.357 g, 31% yield). 1H NMR (500 MHz, cdcl3) δ 10.01 (s, 1H), 7.64 (dd, J=5.2, 2.2 Hz, 2H), 6.35 (t, J=2.2 Hz, 1H), 2.52-2.39 (m, 2H), 2.33 (ddd, J=14.6, 10.2, 4.2 Hz, 2H), 1.70 (dq, J=13.3, 6.1 Hz, 2H), 1.52-1.41 (m, 4H).

A 100 mL 24/40 roundbottom flask was charged with a stir bar, 1-(1-Pyrazolyl)cyclohexanecarboxylic Acid, (0.194 g, 1,00 mmol), triethylamine (0.303 g, 3.00 mmol), N,N-dimethylformamide (2.00 mL), and 1-[Bis(dimethylamino)methylene]-1H-1,2,3-triazolo[4,5-b]pyridinium 3-oxide hexafluorophosphate (0.418 g, 1.10 mmol). The reaction vessel was sealed, flushed with argon, and 5,6-dichloro-benzoildFirnidazol-2-amine (0.242 g, 1.20 mmol) was added dissolved in N,N-dimethylformamide (1.00 mL). The reaction mixture was stirred for 20 hours, quenched with ethyl acetate, washed with brine, aqueous sodium bicarbonate, and brine. The organic layer was dried over sodium sulfate, filtered, and concentrated in vacuo. This material was purified by Reverse-phase Medium Pressure Liquid Chromatography (50 g C18 column, methanol in 25.0 mmolar aqueous ammonium formate). This material was additionally purified by silica gel chromatography (0 to 10% methanol in dichloromethane) The product was then partitioned between ethyl acetate and saturated sodium bicarbonate solution, washed with brine, dried over sodium sulfate, filtered and concentrated in vacuo. This material was recrystallized from dichloromethane/hexanes to obtain the pure product (0.171 g, 45% yield), 1H NMR (500 MHz, Chloroform-d) δ 10.84 (s, 1H), 9.37 (s, 1H), 7.80 (dd, J=1.9, 0.6 Hz, 1 H), 7,69 (dd, J=2.5, 0.6 Hz, 1H), 7.53 (d, J=69.9 Hz, 2H), 6.46 (dd, J=2.5, 1.9 Hz, 1H), 2.69 (d, J=14.3 Hz, 2H), 2.33 (td, J=14.0, 13,2, 3.9 Hz, 2H), 1.84-1.75 (m, 2H), 1.66 (dd, J=12.8, 3.9 Hz, 1H), 1.50-1.29 (m, 3H), HRMS ESI (+) Calc'd for [M+H]=378.0884, found=378.0896.

### Example 64

### N-(5,6-dichloro-1H-benzo[d]imidazol-2-yl)-1-phenylcyclopentane-1-carboxamide

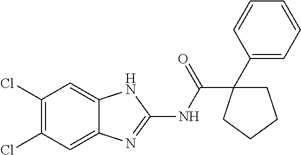 [SMILES: O=C(Nc1nc2cc(Cl)c(Cl)cc2[nH]1)C1(c2ccccc2)CCCC1]

A 100 mL 24/40 roundbottom flask was charged with a stir bar, 1-phenyl-1-cyclopentyl carboxylic acid (0.152 g, 0,800 mmol), N,N-dimethylformamide (2.00 mL), triethylamine (0.202 g, 2.00 mmol), and 1-[Bis(dimethylamino)methlene]-1H-1,2,3-triazolo[4,5-b]pyridinium 3-oxide hexafluorophosphate (0.342 g, 0.900 mmol). The reaction vessel was sealed, flushed with argon and 5,6-dichloro-1H-benzo[d]imiciazole-2-amine (0.202 g, 1.00 mmol) was added dissolved in N,N-dimethylformamide (1.00 mL). The reaction mixture was stirred for 20 hours, quenched with ethyl acetate, washed with brine, aqueous sodium bicarbonate, and brine. This material was purified by Reverse-phase Medium Pressure Liquid Chromatography (50 g C18 column, methanol in 25.0 mrnolar aqueous ammonium formate). The product was then partitioned between ethyl acetate and saturated sodium bicarbonate solution, washed with brine, dried over sodium sulfate, filtered and concentrated in vacuo. This material was additionally purified by silica gel chromatography (0 to 10% methanol in dichloromethane). The pure product was obtained as a white powder by recrystallization from dichloromethane/hexanes (0.108 g, 29% yield). 1H NMR (500 MHz, DMSO-d6) δ 12.38 (s, 1H), 11.32 (s, 1H), 7.58 (s, 2H), 7.46-7.39 (m, 2H), 7.34 (t, J=7.6 Hz, 2H), 7.24 (t, J=7.3 Hz, 1H), 2.69 (dd, J=11.8, 6.7 Hz, 2H), 2.01-1.92 (m, 2H), 1.73 1.58 (m, 4H). HRMS ESI (+) Calc'd for [M+H]=374.0823, found=374.0837.

### Example 65: N-(5,6-dichloro-1H-benzo[d]imidazol-2-yl)-1-phenylcyclopropane-1-carboxamide

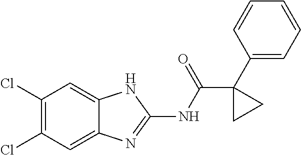 [SMILES: O=C(Nc1nc2cc(Cl)c(Cl)cc2[nH]1)C1(c2ccccc2)CC1]

A 100 mL 24/40 roundbottom flask was charged with a stir bar, 1-phenyl-1-cyclopropyl carboxylic acid (0.162 g, 1.00 mmol), N,N-dimethylformamide (2.00 mL), triethylamine (0.202 g, 2.00 mmol), and 1-[Bis(dimethylamino)methylene]-1,2,3-triazolo[4,5-b]pyridinium 3-oxide hexafluorophosphate (0.418 g, 1.10 mmol). The reaction vessel was sealed, flushed with argon and 5,6-dichloro-1H-benzo[d]imidazole-2-amine (0.242 g, 1.20 mmol) was added dissolved in N,N-dimethylformamide (1.00 mL). The reaction mixture was stirred for 20 hours, quenched with ethyl acetate, washed with brine, aqueous sodium bicarbonate, and brine. This material was purified by Reverse-phase Medium Pressure Liquid Chromatography (50 g C18 column, methanol in 25.0 mmolar aqueous ammonium formate). The product was then partitioned between ethyl acetate and saturated sodium bicarbonate solution, washed with brine, dried over sodium sulfate, filtered and concentrated in vacuo. This material was additionally purified by silica gel chromatography (0 to 10% methanol in dichioromethane). The pure product was obtained as a white powder by recrystallization from ethyl acetate/hexanes (0.122 g, 34%). 1H NMR (500 MHz, DMSO-d6) δ 12.36 (s, 1H), 10.80 (s, 1H), 7.59 (s, 2H), 7.43-7.39 (m, 2H), 7.35 (t, J=7.5 Hz, 2H), 7.29 (t, J=7.2 Hz, 1H), 1.61-1.49 (m, 2H), 1.21 (q, J=4.4 Hz, 2H). HRMS ESI (+) Calc'd for [M+H]=346.0510, found=346.0522.

### Example 66

### N-(5,6-dichloro-1H-benzo[d]imidazol-2-yl)-4-methyltetrahydro-2H-pyran-4-carboxamide

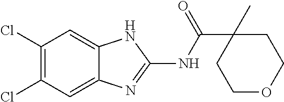 [SMILES: CC1(C(=O)Nc2nc3cc(Cl)c(Cl)cc3[nH]2)CCOCC1]

A 100 mL 24/40 roundbottom flask was charged with a stir bar, 4-methyl-tetrahydro-2H-pyran-4-carboxylic acid (0.144 g, 1.00 mmol), N,N-dimethylformamide (2.00 mL), triethylamine (0.202 g, 2.00 mmol), and 1-[Bis(dimethylamino)methylene]-1H-1,2,3-triazolo[4,5-b]pyridinium 3-oxide hexafluorophosphate (0.418 g, 1.10 mmol). The reaction vessel was sealed, flushed with argon and 5,6-dichloro-1H-benzo[d]imidazole-2-amine (0.242 g, 1.20 mmol) was added dissolved in N,N-dimethylformamide (1.00 mL). The reaction mixture was stirred for 20 hours, quenched with ethyl acetate, washed with brine, aqueous sodium bicarbonate, and brine. This material was purified by Reverse-phase Medium Pressure Liquid Chromatography (50 g C18 column, methanol in 25.0 mmolar aqueous ammonium formate). The product was then partitioned between ethyl acetate and saturated sodium bicarbonate solution, washed with brine, dried over sodium sulfate, filtered and concentrated in vacuo. This material was purified by silica gel chromatography (0 to 10% methanol in dichioromethane) to obtain the final product (0.131 g, 40% yield). 1H NMR (500 MHz, DMSO-d6) δ 12.41 (s, 1H), 11.46 (5, 1H), 7.64 (s, 1H), 7.62 (s, 1H), 3.67 (dt, J=11.4, 4.5 Hz, 2H), 3.45 (ddd, J=11.8, 9.1, 2.7 Hz, 2H), 2.16-2.06 (m, 2H), 1.52 (ddd, J=13.4, 9.0, 3.7 Hz, 2H), 1.29 (s, 3H). HRMS ESI (+) Calc'd for [M+H]=328.0615, found=328.0627.

### Example 67

### N-(5,6-dichloro-1H-benzo[d]imidazol-2-yl)-3-methyloxetane-3-carboxamide

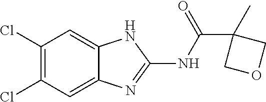 [SMILES: CC1(C(=O)Nc2nc3cc(Cl)c(Cl)cc3[nH]2)COC1]

A 100 mL 24/40 roundbottom flask was charged with a stir bar, 3-methyloxetane-3-carboxylic acid (0.116 g, 1.00 mmol), N,N-dimethylformamide (2.00 mL), triethylamine (0.202 g, 2.00 mmol), and 1-[Bis(dimethylamino)methylene]-1H-1,2,3-triazolo[4,5-b]pyridinium 3-oxide hexafluorophosphate (0.418 g, 1.10 mmol). The reaction vessel was sealed, flushed with argon and 5,6-dichloro-1H-benzo[d]imidazole-2-amine (0.249 g, 1.20 mmol) was added dissolved in N,N-dimethylformamide (1.00 mL). The reaction mixture was stirred for 22 hours, quenched with ethyl acetate, washed with brine, aqueous sodium bicarbonate, and brine. This material was purified by Reverse-phase Medium Pressure Liquid Chromatography (50 g C18 column, methanol in 25.0 mmolar aqueous ammonium formate). The product was then partitioned between ethyl acetate and saturated sodium bicarbonate solution, washed with brine, dried over sodium sulfate, filtered and concentrated in vacuo. This material was purified by silica gel chromatography (0 to 10% methanol in dichloromethane). The final product was obtained by recrystallizing the material from ethyl acetate/hexanes (0.107 g, 36% yield). 1H NMR (500 MHz, DMSO-d6) δ 12.39 (s, 1H), 11.82 (s, 1H), 7.66 (d, J=9.4 Hz, 2H), 4.85 (d, J=6.2 Hz, 2H), 4.37 (d, J=6.3 Hz, 2H), 1.63 (s, 3H). HRMS ESI (+) Calc'd for [M+]=300.0302, found=300.0315.

### Example 68

### N-(5,6-dichloro-1H-benzo[d]imidazol-2-yl-1-ethoxycyclopropane-1-carboxamide

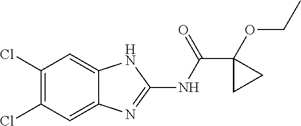 [SMILES: CCOC1(C(=O)Nc2nc3cc(Cl)c(Cl)cc3[nH]2)CC1]

A 100 mL 24/40 roundbottom flask was charged with a stir bar, 1-ethylcyclopropane-1-carboxylic acid (0.241 g, 1.85 mmol), N,N-dimethylformamide (2.00 mL), triethylamine (0.404 g, 4.00 mmol), and 1-[Bis(dimethylamino)methylene]H-1,2,3-triazolo[4,5-b]pyridinium 3-oxide hexafluorophosphate (0.741 g, 1.95 mmol). The reaction vessel was sealed, flushed with argon and 5,6-dichloro-1H-benzo[d]imidazole-2-amine (0.414 g, 2.05 mmol) was added dissolved in N,N-dimethylformamide (2.00 mL). The reaction mixture was stirred for 22 hours, quenched with ethyl acetate, washed with brine, aqueous sodium bicarbonate, and brine. This material was purified by Reverse-phase Medium Pressure Liquid Chromatography (50 g C18 column, methanol in 25.0 mmolar aqueous ammonium formate). The product was then partitioned between ethyl acetate and saturated sodium bicarbonate solution, washed with brine, dried over sodium sulfate, filtered and concentrated in vacuo. This material was purified by silica gel chromatography (0 to 10% methanol in dichloromethane). The final product was obtained by recrystallizing the material from dichloromethane/hexanes (0.153 g, 26% yield). 1H NMR (500 MHz, DMSO-d6) δ 12.38 (s, 1H), 11.38 (s, 1H), 7.65 (s, 2H), 3.56 (q, J=6.9 Hz, 2H), 1.30-1.25 (m, 2H), 1.24 1.20 (m, 2H), 1.17 (t, J=7.0 Hz, 3H). HRMS ESI (+) Calc'd for [M+H]=314.0459, found=314.0467.

### Example 69: N-(5,6-dichloro-1H-benzo[d]irnidazol-2-yl)-2-methoxy-2-methylpropanamide

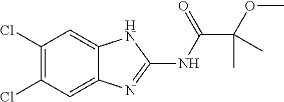 [SMILES: COC(C)(C)C(=O)Nc1nc2cc(Cl)c(Cl)cc2[nH]1]

A 100 mL 24/40 roundbottom flask was charged with a stir bar, 2-methoxy-2-methyl propionic acid (0.129 g, 1.10 mmol), N,N-dimethylformamide (2.00 mL), triethylamine (0.202 g, 2.00 mmol), and 1-[Bis(dimethylamino)methylene]-1H-1,2,3-triazolo[4,5-b]pyridinium 3-oxide hexafiuorophosphate (0.456 g, 1.20 mmol) The reaction vessel was sealed, flushed with argon and 5,6-dichloro-1H-1-benzo[d]imidazole-2-amine (0.262 g, 1.30 mmol) was added dissolved in N,N-dimethylformamide (1.00 mL). The reaction mixture was stirred for 22 hours, quenched with ethyl acetate, washed with brine, aqueous sodium bicarbonate, and brine. This material was purified by Reverse-phase Medium Pressure Liquid Chromatography (50 g C18 column, methanol in 25.0 mmolar aqueous ammonium formate). The product was then partitioned between ethyl acetate and saturated sodium bicarbonate solution, washed with brine, dried over sodium sulfate, filtered and concentrated in vacuo. This material was purified by silica gel chromatography (0 to 10% methanol in dichloromethane). The final product was obtained by recrystallizing the material from dichloromethane/hexanes (0.076 g, 22% yield). 1H NMR (500 MHz, DMSO-d6) δ 12.39 (s, 1H), 11.22 (s, 1H), 7.65 (d, J=17.0 Hz, 2H), 3,20 (s, 3H), 1.40 (s, 6H). HRMS ESI (+) Calc'd for [M+H]=302.0459, found=302.0471.

The disclosure provides for the following example embodiments, the numbering of which is not to be construed as designating levels of importance:

Embodiment 1 relates to a compound of the formula (I) or (I′):

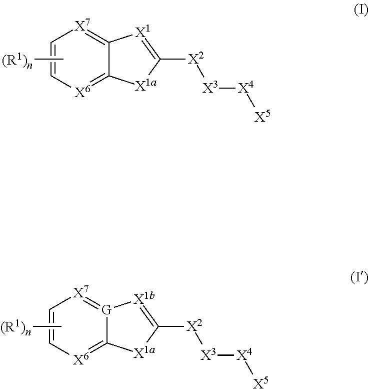

or a pharmaceutically acceptable salt, polymorph, prodrug, solvate or clathrate thereof wherein:
each R¹ is independently H, halo, alkyl, alkoxy, aryl, aryloxy, amino, S(O)ₙR¹, nitro, cyano, heterocyclyl or two groups on adjacent atoms can form an aryl or a heterocyclyl group together with the atoms to which they are attached;
n is 0, 1, or 2;
X¹ and X^{1a} are each independently O, N or NR⁴, wherein R⁴ is H, alkyl, cycloalkyl or aryl;
is N or C;
X^{1b} is N or CH;
X² is alkyl, alkenyl, O, NR^{4 a}, C(O) or S(O)ₓ, wherein x is 0, 1 or 2 and R^{4a} is H, alkyl, cycloalkyl or aryl;
X³ is absent, alkyl or NR⁴;
X⁴ is C(O) or alkyl;
X⁵ is alkyl, cycloalkyl, heterocyclyl or NR²R³, wherein R² and R³ can each independently be H, alkyl, cycloalkyl, aryl or R² and R³, together with the nitrogen atom to which they are attached, form a heterocyclyl group;
or wherein X²-X⁴, together with substituents attached thereto, form a 3-5-membered ring;
X⁶ and X⁷ are each independently N or CR⁵, wherein R⁵ can be H or R¹.

Embodiment 2 relates to the compound of Embodiment 1, wherein X² is S(O)ₜ, wherein t is 1 or 2.

Embodiment 3 relates to the compound of Embodiment 1 or 2, wherein X² is S.

Embodiment 4 relates to the compound of Embodiment 1, wherein X² is NR^{4a}.

Embodiment 5 relates to the compound of Embodiment 1, wherein X² is O.

Embodiment 6 relates to the compound of Embodiment 1, wherein X² is C(O).

Embodiment 7 relates to the compound of Embodiment 1, wherein X² is alkyl.

Embodiment 8 relates to the compound of Embodiment 1, wherein X² is alkenyl.

Embodiment 9 relates to the compound of any preceding Embodiment, wherein X³ is absent or alkyl.

Embodiment 10 relates to the compound of any preceding Embodiment, wherein X³ is (C₁-C₆)alkyl.

Embodiment 11 relates to the compound of any preceding Embodiment, wherein X⁴ is C(O).

Embodiment 12 relates to the compound of any preceding Embodiment, wherein X⁵ is cycloalkyl or NR²R³.

Embodiment 13 relates to the compound of any preceding Embodiment, wherein X⁵ is NR²R³.

Embodiment 14 relates to the compound of any preceding Embodiment, wherein X⁵ is NR²R³ and R² and R³, together with the nitrogen atom to which they are attached, form a heterocyclyl group.

Embodiment 15 relates to the compound of Embodiments 1-13, wherein X⁵ is NR²R³ and at least one of R² and R³ is aryl.

Embodiment 16 relates to the compound of Embodiments 1-131 -13, wherein X⁵ is NR²R³ and at least one of R² and R³ is cycloalkyl.

Embodiment 17 relates to the compound of any preceding Embodiment, wherein the compound is a compound of the formula:

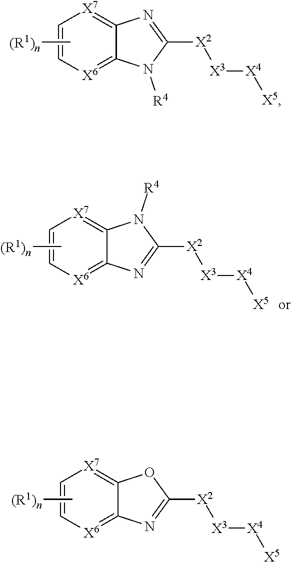

Embodiment 18 relates to the compound of Embodiment 17, wherein R⁴ is alkyl.

Embodiment 19 relates to the compound of Embodiments 1-18, wherein X² is S(O) and X³ is alkyl.

Embodiment 20 relates to the compound of Embodiment 19, wherein X² is S.

Embodiment 21 relates to the compound of Embodiments 1-18, wherein X² is alkyl and X³ is absent.

Embodiment 22 relates to the compound of Embodiments 1-18, wherein X² is alkenyl and X³ is absent.

Embodiment 23 relates to the compound of Embodiments 1-18, wherein X² is alkyl and X³ is NR⁴.

Embodiment 24 relates to the compound of Embodiments 1-18, wherein X² is NR^{4a} and X³ is alkyl.

Embodiment 25 relates to the compound of Embodiments 1-18, wherein X² is NR^{4a} and X³ is absent.

Embodiment 26 relates to the compound of Embodiments 1-18, wherein X² is O and X³ is alkyl.

Embodiment 27 relates to the compound of Embodiments 1-18, wherein X² is C(O) and X³ is absent.

Embodiment 28 relates to the compound of any preceding Embodiment, wherein at least one of X⁶ and X⁷ is N.

Embodiment 29 relates to the compound of any preceding Embodiment, wherein X⁶ is CR⁵ and X⁷ is N, wherein R⁵ can be H or R¹.

Embodiment 30 relates to the compound of Embodiments 1-27, wherein at least one of X⁶ and X⁷ is CR⁵, wherein R⁵ can be H or R¹.

Embodiment 31 relates to the compound of Embodiment 30, wherein X⁶ and X⁷ are each independently CR⁵.

Embodiment 32 relates to a compound of the formula (III):

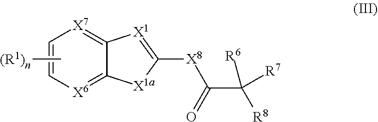

or a pharmaceutically acceptable salt, polymorph, prodrug, solvate or clathrate thereof wherein:
each R¹ is independently H, halo, alkyl, alkoxy, aryl, aryloxy, amino, S(O)ₙR¹, nitro, cyano, heterocyclyi or two R¹ groups on adjacent atoms can form an aryl or a heteracyclyl group together with the atoms to which they are attached;
n is 0, 1, or 2;
X¹ and X^{1a} are each independently O, N or NR⁴, wherein R⁴ is H, alkyl, cycloalkyl or aryl;
X⁶ and X⁷ are each independently N or CR⁵, wherein R⁵ can be H or R¹;
X⁸ is NR^{4a}, wherein R^{4a} is H, alkyl, cycloalkyl or aryl;
R⁶ and R⁷ are each, independently, alkyl or, together with the carbon atom to which they are attached, form a cycloalkyl or a heterocyclyl; and
R⁸ is alkyl, alkoxy, aryl or heteroaryl.

Embodiment 33 relates to a compound of the formula (IV):

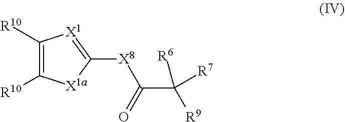

or a pharmaceutically acceptable salt, polymorph, prodrug, solvate or clathrate thereof wherein:
X¹ and X^{1a} are each independently O, N or NR⁴, wherein R⁵ is H, alkyl, cycloalkyl or aryl;
X⁶ and X⁷ are each independently N or CR⁵, wherein R⁵ can be H or R¹;
X⁸ is NR^{4a}, wherein R^{4a} is H, alkyl, cycloalkyl or aryl;
R⁶ and R⁷ are each, independently, alkyl or, together with the carbon atom to which they are attached, form a cycloalkyl or a heterocyclyl;
R⁹ is alkyl (e.g., methyl and ethyl), haloalkyl (e.g., CF₃), alkoxy, haloalkoxy (e.g., OCF₃), aryloxy, cycloalkyl (e.g., (C₃-C₆)cycloalkyl and (C₃-C₅)cycloalkyl, such as cyclopropyl, cyclobutyl, cyclopentyl and cyclohexyl) or heteroaryl; and
each R¹⁰ is, independently, alkyl, aryl or arylalkyl.

Embodiment 34 relates to a pharmaceutical composition comprising one or more compounds of Embodiments 1-33 and one or more pharmaceutically acceptable carriers, diluents, excipients or combinations thereof.

Embodiment 35 relates to a method for treating at least one of tuberculosis and a nontuberculous mycobacteria infection, the method comprising administering one or more compounds of Embodiments 1-32 or the pharmaceutical composition of claim 34 to a subject in need of treatment of at least one of tuberculosis and a nontuberculous mycobacteria infection.

1. A compound of the formula (I) or (I′): 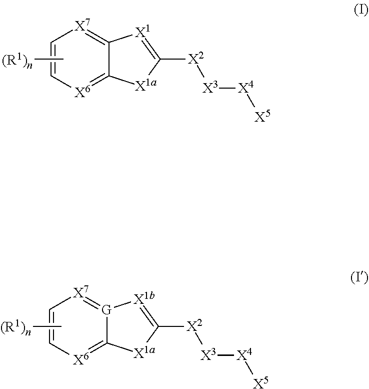
or a pharmaceutically acceptable salt, polymorph, prodrug, solvate or clathrate thereof wherein:
each R¹ is independently H, halo, alkyl, alkoxy, aryl, aryloxy, amino, S(O)ₙR¹, nitro, cyano, heterocyclyl or two R¹ groups on adjacent atoms can form an aryl or a heterocyclyl group together with the atoms to which they are attached;
n is 0, 1, or 2;
X¹ and X^{1a} are each independently O, N or NR⁴, wherein R⁴ is H, alkyl, cycloalkyl or aryl;
G is N or C;
X^{1b} is N or CH;
X² is alkyl, alkenyl, O, NR^{4a}, C(O) or S(O)ₓ, wherein x is 0, 1 or 2 and R^{4a} is H, alkyl, cycloalkyl or aryl;
X³ is absent, alkyl or NR⁴;
X⁴ is C(O) or alkyl;
X⁵ is alkyl, cycloalkyl, heterocyclyl or NR²R³, wherein R² and R³ can each independently be H, alkyl, cycloalkyl, aryl or R² and R³, together with the nitrogen atom to which they are attached, form a heterocyclyl group;
or wherein X²-X⁴, together with substituents attached thereto, form a 3-5-membered ring;
X⁶ and X⁷ are each independently N or CR⁵, wherein R⁵ can be H or R¹.

2. The compound of claim 1, wherein X² is S(O)ₓ, wherein x is 1 or 2.

3. The compound of claim 1, wherein X² is S, NR^{4a} or O.

4. (canceled)

5. (canceled)

6. The compound of claim 1, wherein X² is C(O).

7. The compound of claim 1, wherein X² is alkyl or alkenyl.

8. (canceled)

9. The compound of claim 1, wherein X³ is absent or (C₁-C₆)alkyl.

10. (canceled)

11. The compound of claim 1, wherein, wherein X⁴ is C(O).

12. The compound of claim 1, wherein X⁵ is cycloalkyl or NR²R³.

13. (canceled)

14. The compound of claim 1, wherein X⁵ is NR²R³ and R² and R³, together with the nitrogen atom to which they are attached, form a heterocyclyl group; X⁵ is NR²R³ and at least one of R² and R³ is aryl, or X⁵ is NR²R³ and at least one of R² and R³ is cycloalkyl.

15. (canceled)

16. (canceled)

17. The compound of claim 1, wherein the compound is a compound of the formula:

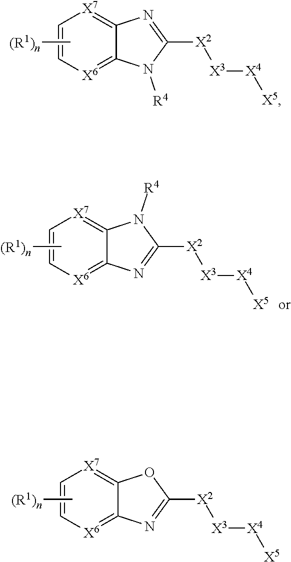

18. The compound of claim 17, wherein R⁴ is alkyl.

19. The compound of claim 17, wherein X² is S(O)ₓ and X³ is alkyl.

20. (canceled)

21. The compound of claim 1, wherein X² is alkyl and X³ is absent X² is alkenyl and X³ is absent X² is alkyl and X³ is NR^{4a}; X² is NR^{4a} and X³ is alkyl; X² is NR^{4a} and X³ is absent; X² is O and X³ is alkyl; or X² is C(O) and X³ is absent.

22. (canceled)

23. (canceled)

24. (canceled)

25. (canceled)

26. (canceled)

27. (canceled)

28. The compound of claim 1, wherein at least one of X⁶ and X⁷ is N; at least one of X⁶ and X⁷ is CR⁵, wherein R⁵ can be H or R¹; or X⁶ is CR⁵ and X⁷ is N, wherein R⁵ can be H or R¹.

29. (canceled)

30. (canceled)

31. The compound of claim 28, wherein X⁶ and X⁷ are each independently CR⁵.

32. A compound of the formula (III): 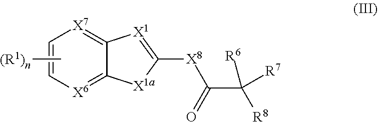
or a pharmaceutically acceptable salt, polymorph, prodrug, solvate or clathrate thereof wherein:
each R¹ is independently H, halo, alkyl, alkoxy, aryl, aryloxy, amino, S(O)ₙR¹, nitro, cyano, heterocyclyl or two R¹ groups on adjacent atoms can form an aryl or a heterocyclyl group together with the atoms to which they are attached;
n is 0, 1, or 2;
X¹ and X^{1a} are each independently O, N or NR⁴, wherein R⁴ is H, alkyl, cycloalkyl or aryl;
X⁶ and X⁷ are each independently N or CR⁵, wherein R⁵ can be H or R¹;
X⁸ is NR^{4a}, wherein R^{4a} is H, alkyl, cycloalkyl or aryl;
R⁶ and R⁷ are each, independently, alkyl or, together with the carbon atom to which they are attached, form a cycloalkyl or a heterocyclyl; and
R⁸ is alkyl, alkoxy, aryl or heteroaryl.

33. A compound of the formula (IV): 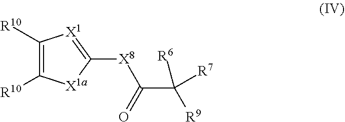
or a pharmaceutically acceptable salt, polymorph, prodrug, solvate or clathrate thereof wherein:
X¹ and X^{1a} are each independently O, N or NR⁴, wherein R⁴ is H, alkyl, cycloalkyl or aryl;
X⁶ and X⁷ are each independently N or CR⁵, wherein R⁵ can be H or R¹;
X⁸ is NR^{4a}, wherein R^{4a} is H, alkyl, cycloalkyl or aryl;
R⁶ and R⁷ are each, independently, alkyl or, together with the carbon atom to which they are attached, form a cycloalkyl or a heterocyclyl;
R⁹ is alkyl (e.g., methyl and ethyl), haloalkyl (e.g., CF₃), alkoxy, haloalkoxy (e.g., OCF₃), aryloxy, cycloalkyl or heteroaryl; and
each R¹⁰ is, independently, alkyl, aryl or arylalkyl.

34. A pharmaceutical composition comprising one or more compounds of claims 1 and one or more pharmaceutically acceptable carriers, diluents, excipients or combinations thereof.

35. A method for treating at least one of tuberculosis and a nontuberculous mycobacteria infection, the method comprising administering one or more compounds of claims 1 to a subject in need of treatment of at least one of tuberculosis and a nontuberculous mycobacteria infection.

36. A compound of claim 1, or a pharmaceutically acceptable salt, polymorph, prodrug, solvate or clathrate thereof wherein the compound is a compound of the formula: 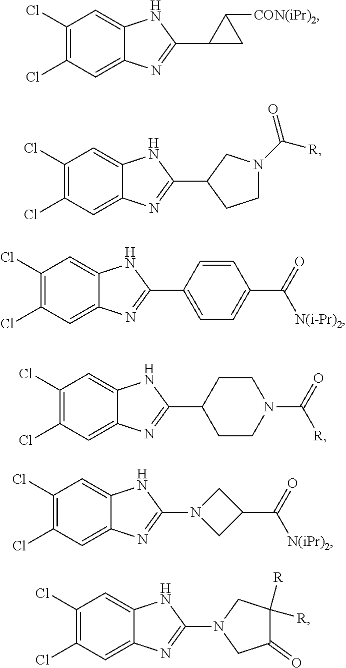 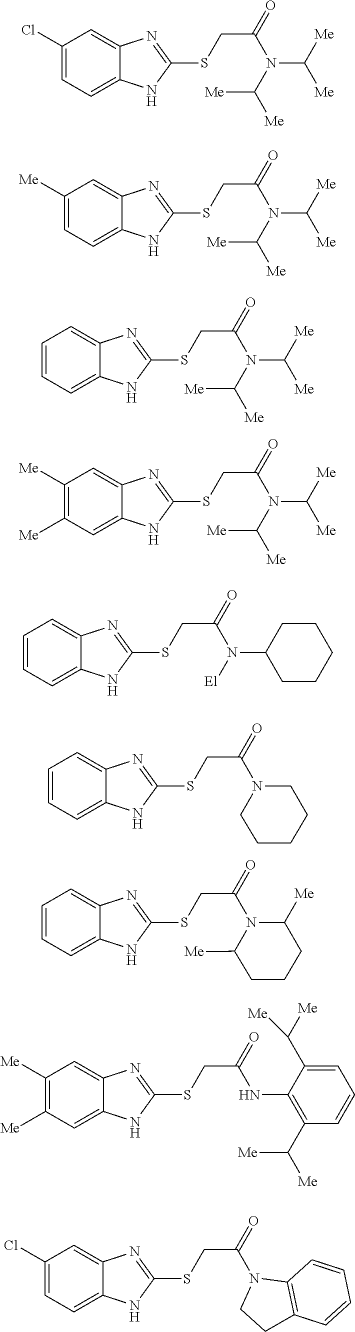 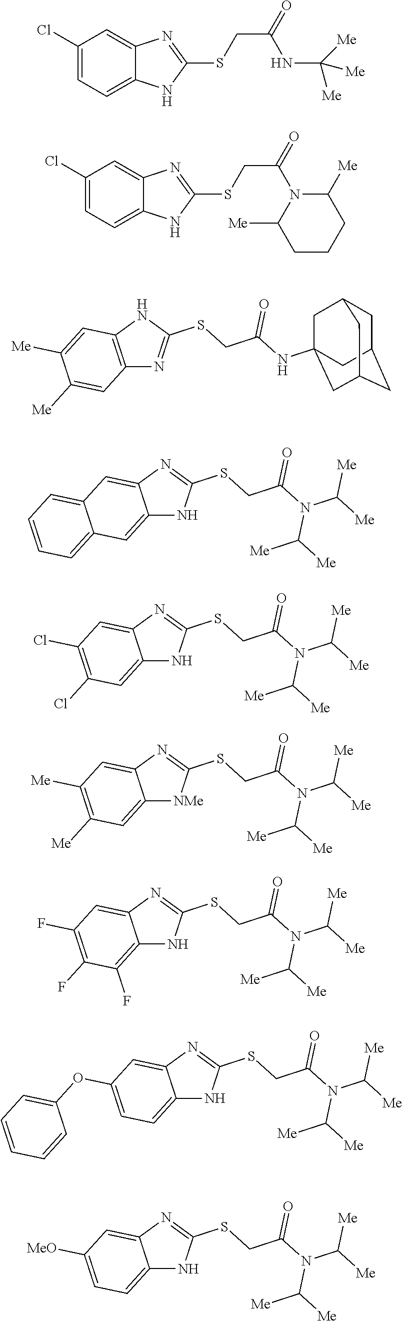 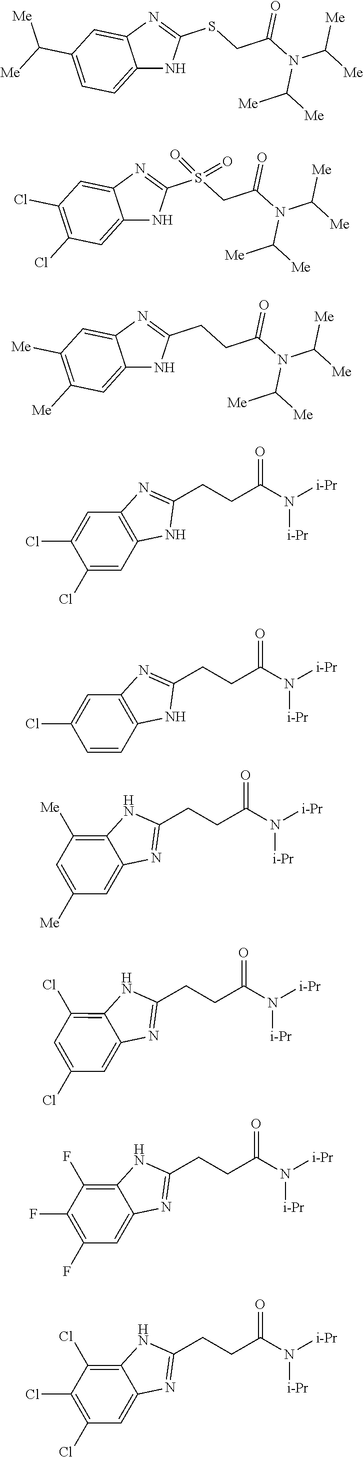 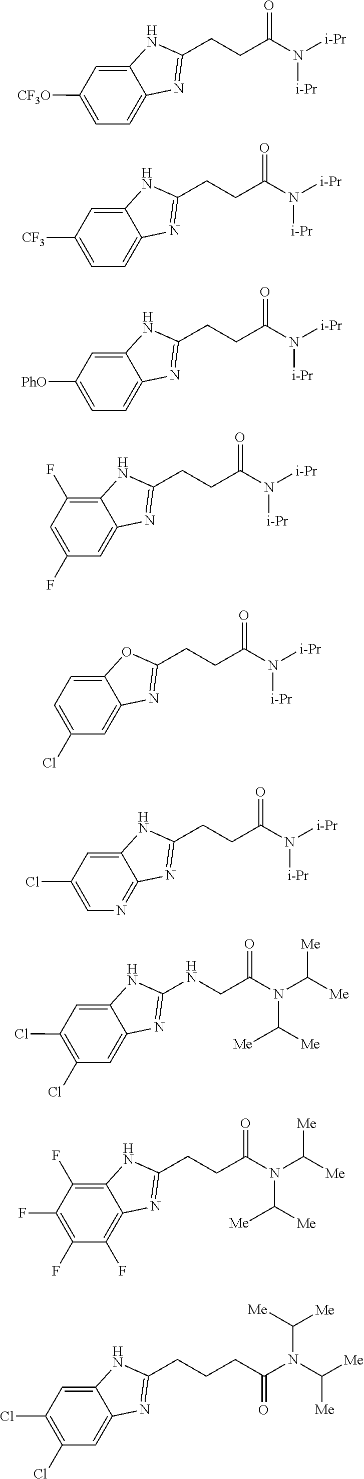 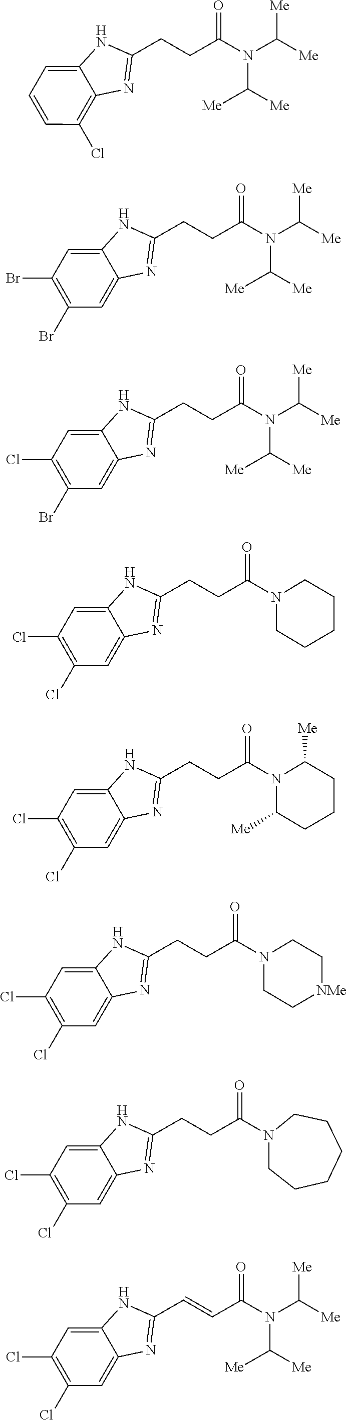 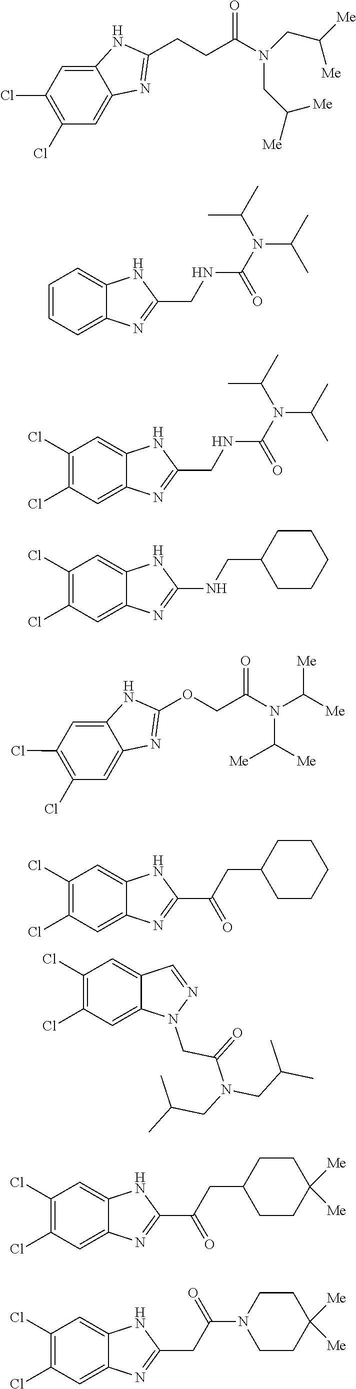 [chemical structure]
